SEQUENCE LISTING (35 sequences)

SEQ ID NO: 1 - protein, 37 aa, Artificial Sequence
  Description of Artificial Sequence synthetic peptide
HDEFERHAEGTFTSDVSSYLEGQAAKEFIAWLVKGRG

SEQ ID NO: 2 - protein, 31 aa, Artificial Sequence
  Description of Artificial Sequence synthetic peptide
HAEGTFTSDVSSYLEGQAAKEFIAWLVKGRG

SEQ ID NO: 3 - protein, 20 aa, Artificial Sequence
  VARIANT 17: Xaa = Lys or Arg
SYLEGQAAKEFIAWLVXGRX

SEQ ID NO: 4 - protein, 21 aa, Artificial Sequence
  VARIANT 18: Xaa = Lys or Arg
SSYLEGQAAKEFIAWLVXGRX

SEQ ID NO: 5 - protein, 22 aa, Artificial Sequence
  VARIANT 19: Xaa = Lys or Arg
VSSYLEGQAAKEFIAWLVXGRX

SEQ ID NO: 6 - protein, 23 aa, Artificial Sequence
  VARIANT 20: Xaa = Lys or Arg
DVSSYLEGQAAKEFIAWLVXGRX

SEQ ID NO: 7 - protein, 24 aa, Artificial Sequence
  VARIANT 21: Xaa = Lys or Arg
SDVSSYLEGQAAKEFIAWLVXGRX

SEQ ID NO: 8 - protein, 25 aa, Artificial Sequence
  VARIANT 22: Xaa = Lys or Arg
TSDVSSYLEGQAAKEFIAWLVXGRX

SEQ ID NO: 9 - protein, 26 aa, Artificial Sequence
  VARIANT 23: Xaa = Lys or Arg
FTSDVSSYLEGQAAKEFIAWLVXGRX

SEQ ID NO: 10 - protein, 27 aa, Artificial Sequence
  VARIANT 24: Xaa = Lys or Arg
TFTSDVSSYLEGQAAKEFIAWLVXGRX

SEQ ID NO: 11 - protein, 28 aa, Artificial Sequence
  VARIANT 25: Xaa = Lys or Arg
GTFTSDVSSYLEGQAAKEFIAWLVXGRX

SEQ ID NO: 12 - protein, 29 aa, Artificial Sequence
  VARIANT 26: Xaa = Lys or Arg
EGTFTSDVSSYLEGQAAKEFIAWLVXGRX

SEQ ID NO: 13 - protein, 30 aa, Artificial Sequence
  VARIANT 27: Xaa = Lys or Arg
AEGTFTSDVSSYLEGQAAKEFIAWLVXGRX

SEQ ID NO: 14 - protein, 31 aa, Artificial Sequence
  VARIANT 28: Xaa = Lys or Arg
HAEGTFTSDVSSYLEGQAAKEFIAWLVXGRX

SEQ ID NO: 15 - protein, 31 aa, Artificial Sequence
  VARIANT 2: Xaa = D-Ala
HXEGTFTSDVSSYLEGQAAKEFIAWLVXGRX

SEQ ID NO: 16 - protein, 39 aa, Artificial Sequence
  Description of Artificial Sequence synthetic peptide
HSDGTFTSDLSKQMEEEAVRLFIEWLKNGGPSSGAPPPS

SEQ ID NO: 17 - protein, 39 aa, Artificial Sequence
  Description of Artificial Sequence synthetic peptide
HGEGTFTSDLSKQMEEEAVRLFIEWLKNGGPSSGAPPPS

SEQ ID NO: 18 - protein, 31 aa, Artificial Sequence
  Description of Artificial Sequence synthetic peptide
HGEGTFTSDLSKQMEEAVRLFIEWLKNGGPY

SEQ ID NO: 19 - protein, 31 aa, Artificial Sequence
  Description of Artificial Sequence synthetic peptide
HGEGTFTSDLSKQMEEEAVRLFIEWLKNGGY

SEQ ID NO: 20 - protein, 31 aa, Artificial Sequence
  Description of Artificial Sequence synthetic peptide
DLSKQMEEEAVRLFIEWLKNGGPSSGAPPPS

SEQ ID NO: 21 - protein, 37 aa, Artificial Sequence
  Description of Artificial Sequence synthetic peptide
HDEFERHAEGTFTSDVSSYLEGQAAKEFIAWLVKGRK

SEQ ID NO: 22 - protein, 31 aa, Artificial Sequence
  Description of Artificial Sequence synthetic peptide
HAEGTFTSDVSSYLEGQAAKEFIAWLVKGRK

SEQ ID NO: 23 - protein, 40 aa, Artificial Sequence
  Description of Artificial Sequence synthetic peptide
HGEGTFTSDLSKQMEEEAVRLFIEWLKNGGPSSGAPPPSK

SEQ ID NO: 24 - protein, 40 aa, Artificial Sequence
  Description of Artificial Sequence synthetic peptide
HSDGTFTSDLSKQMEEEAVRLFIEWLKNGGPSSGAPPPSK

SEQ ID NO: 25 - protein, 31 aa, Artificial Sequence
  Description of Artificial Sequence synthetic peptide
HGEGTFTSDLSKEMEEEVRLFIEWLKNGGPY

SEQ ID NO: 26 - protein, 30 aa, Artificial Sequence
  Description of Artificial Sequence synthetic peptide
HGEGTFTSDLSKEMEEEVRLFIEWLKNGGY

SEQ ID NO: 27 - protein, 29 aa, Artificial Sequence
  Description of Artificial Sequence synthetic peptide
DLSKQMEEEAVRLFIEWLKGGPSSGPPPS

SEQ ID NO: 28 - protein, 39 aa, Artificial Sequence
  Description of Artificial Sequence synthetic peptide
HAEGTFTSDVSSYLEGQAAKEFIAWLVKGRPSSGAPPPS

SEQ ID NO: 29 - protein, 640 aa, Artificial Sequence
  Description of Artificial Sequence synthetic peptide
HGEGTFTSDVSSYLEEQAAKEFIAWLVKGRGSSGAPPPSGGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 30 - protein, 585 aa, Homo sapiens [sequence omitted]

SEQ ID NO: 31 - protein, 40 aa, Artificial Sequence
  BINDING 40: Xaa represents Lys (E-AEEA-MPA)-NH2 conjugated with S, O or NH of a protein, and where “E” represents Epsilon
HGEGTFTSDLSKQMEEEAVRLFIEWLKNGGPSSGAPPPSX

SEQ ID NO: 32 - protein, 31 aa, Artificial Sequence
  SITE 2: Xaa represents D-Ala
HXEGTFTSDVSSYLEGQAAKEFIAWLVKGRX

SEQ ID NO: 33 - protein, 40 aa, Artificial Sequence; identical to SEQ ID NO: 31
  BINDING 40: Xaa represents Lys (E-AEEA-MPA)-NH2 conjugated with S, O or NH of albumin, and where “E” represents Epsilon

SEQ ID NO: 34 - protein, 40 aa, Artificial Sequence; identical to SEQ ID NO: 31
  BINDING 40: Xaa represents Lys (E-AEEA-MPA)-NH2 conjugated with S of cysteine 34 of albumin, and where “E” represents Epsilon

SEQ ID NO: 35 - protein, 40 aa, Artificial Sequence; identical to SEQ ID NO: 31
  BINDING 40: Xaa represents Lys (E-AEEA-MPA)-NH2 where “E” represents Epsilon

(19) United States
(12) Patent Application Publication
(10) Pub. No.: US 2009/0186819 A1
(43) Pub. Date: Jul. 23, 2009

(54) FORMULATION OF INSULINOTROPIC PEPTIDE CONJUGATES

(76) Inventors: Marieve Carrier, Longueuil (CA); Byeong Seon Chang, Thousand Oaks, CA (US); Omar Quraishi, Westmont (CA); Thomas R. Ulich, New York, NY (US); Maggie Wang, Montreal (CA); Jean-Philippe Estradier, Montreal (CA)

Correspondence Address: JONES DAY, 222 EAST 41ST ST, NEW YORK, NY, 10017, US

(21) Appl. No.: 12/333,276

(22) Filed: Dec. 11, 2008

Related U.S. Application Data
(60) Provisional application No. 61/007,346, filed on Dec. 11, 2007.
(60) Provisional application No. 61/029,295, filed on Feb. 15, 2008.
(60) Provisional application No. 61/200,879, filed on Dec. 3, 2008.

(51) Int. Cl. A61K 38/38; A61P 3/10

(52) U.S. Cl. 514/12

(57) ABSTRACT

The present invention provides pharmaceutical formulations comprising insulinotropic peptide conjugates, particularly a conjugate of albumin to exendin-4, or a derivative thereof, and methods of administration thereof. The present invention also provides methods for treating diabetes and insulinotropic peptides related diseases or conditions by administering the pharmaceutical formulations described herein.

This application claims benefit of U.S. Provisional Application No. 61/007,346, filed Dec. 11, 2007, of U.S. Provisional Application No. 61/029,295, filed Feb. 15, 2008, and of U.S. Provisional Application No. 61/200,879, filed Dec. 3, 2008, each of which is incorporated by reference herein in its entirety.

### 1. FIELD OF THE INVENTION

Pharmaceutical formulations comprising an insulinotropic peptide conjugate and methods of administration thereof are provided. The formulations are useful in the treatment of diabetes and other insulinotropic peptide related diseases.

### 2. BACKGROUND OF THE INVENTION

The prevalence of diabetes for all age groups worldwide was estimated to be 2.8%, or 171 million in 2000, and is projected to be 4.4%, or 366 million in 2030. See Wild et al., 2004, Diabetes Care 27(5):1047-1053. In the United States alone, the prevalence of diabetes mellitus in 2005 was estimated at 20.8 million, or roughly 7% of the U.S. population. See Centers for Disease Control and Prevention, 2005, National Diabetes Fact Sheet: General Information and National Estimates on Diabetes in the United States, 2005. Approximately 95% of all subjects with diabetes mellitus have type II disease. Diabetes is currently the fifth leading cause of death in the United States and is associated with excess morbidity stemming from cardiovascular disease, kidney failure, blindness, and lower limb amputation.

Similarly, obesity is a condition increasingly affecting the population worldwide. According to the World Health Organization, in 1995 there were an estimated 200 million obese adults worldwide and another 18 million under-five children classified as overweight. As of 2000, the number of obese adults had increased to over 300 million. See Formiguera et al., 2004, Best Practice & Research Clinical Gastroenterology, 18:6, 1125-1146.

The insulinotropic peptide has been investigated as a possible therapeutic agent for the management of type II non-insulin-dependent diabetes mellitus as well as related metabolic disorders, such as obesity. Recently, it has been shown that conjugation of insulinotropic peptides to albumin can provide longer duration of action in vivo while maintaining their low toxicity and therapeutic advantages. See, e.g., Giannoukakis, Curr Opin Investig Drugs. 4(10):1245-9 (2003). Formulations of such pharmaceutical products can be useful for providing stability and maintaining effectiveness. Thus, there is a need in the art for pharmaceutical formulations comprising insulinotropic peptide conjugates.

### 3. SUMMARY OF THE INVENTION

Provided herein are pharmaceutical formulations capable of providing stability and maintaining the biological activity of insulinotropic peptide conjugates. The pharmaceutical formulations provided herein include liquid and lyophilized formulations, unit dosage forms and multi-use dosage forms, and combinations thereof. The pharmaceutical formulations can be suitable for administration via parenteral routes such as subcutaneous, intravenous, intramuscular, transdermal, intra-arterial, intra-peritoneal, or via oral routes, topical routes, or inhalation routes etc.

In one aspect, provided herein are pharmaceutical formulations comprising an insulinotropic peptide conjugate, a buffer, a tonicity modifier, a stabilizer, a surfactant and optionally a preservative, wherein said formulation has a pH of about 3.0 to 8.0. In some embodiments, the formulation has a pH of about 4.0 to 8.0. In some embodiments, the formulation has a pH of about 4.0 to 6.0. In some embodiments, the formulation has a pH of about 6.0 to 8.0. In some embodiments, the formulation has a pH of about 6.0 to 9.0. In some embodiments, the formulation has a pH of about 5.0 to 7.0. In some embodiments, the formulation has a pH of about 4.5 to 6.0. In some embodiments, the formulation has a pH of about 5.0 to 6.0. In some embodiments, the formulation has a pH of about 5.1 to 6.0, about 5.2 to 6.0, about 5.3 to 6.0, about 5.4 to 6.0, about 5.5 to 6.0, about 5.6 to 6.0, about 5.7 to 6.0, or about 5.8 to 6.0. In some embodiments, said formulation has a pH of about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9 or 9.0. In a particular embodiment, the formulation has a pH of about 5.0. In another particular embodiment, the formulation has a pH of about 7.0.

The insulinotropic peptide can be any insulinotropic peptide known to those of skill in the art. For example, it can be any peptide that can stimulate, or cause the stimulation of, synthesis or expression of the hormone insulin. In some embodiments, the insulinotropic peptide is selected from the group consisting of glucagon-like peptide 1, exendin-3 and exendin-4 and their precursors, derivatives or fragments. In preferred embodiments, the insulinotropic peptide is exendin-4 or a derivative thereof. Exemplary derivatives are described herein.

The insulinotropic peptide conjugates can be conjugated to albumin. In some embodiments, the insulinotropic peptide is conjugated to human serum albumin. In some embodiments, the insulinotropic peptide is conjugated to recombinant human serum albumin.

In another aspect, provided herein are pharmaceutical formulations comprising an conjugate of albumin to exendin-4, or a derivative thereof, at a concentration from about 1 mg/ml to about 100 mg/ml, a buffer, a tonicity modifier, a stabilizer, a surfactant and optionally a preservative, wherein said formulation has a pH from about 4 to about 8. In preferred embodiments, the conjugate of albumin to exendin-4 is exendin-4(1-39)-Lys⁴⁰ (ε-AEEA-MPA)-NH₂ albumin conjugate. The term “exendin-4(1-39) Lys⁴⁰ (ε-AEEA-MPA)-NH₂ albumin conjugate” refers to a conjugate made by covalently bonding a compound of the formula:

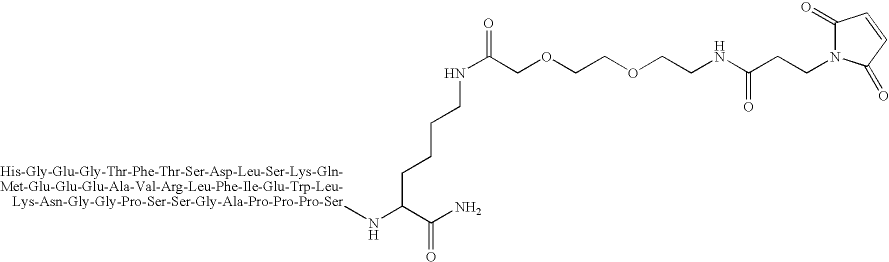

(SEQ ID NO: 35) to albumin, which results in a conjugate of the formula:

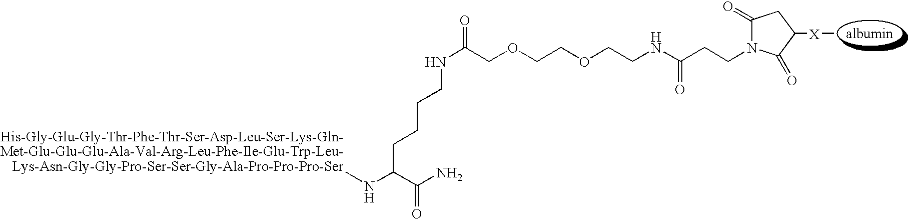

(SEQ ID NO:34) wherein X is the sulfur atom of cysteine 34 of albumin. Those of skill in the art will recognize that exendin-4(1-39) Lys⁴⁰ (ε-AEEA-MPA)-NH₂ albumin conjugate can be formed by covalently linking the cysteine 34 side chain thiol of albumin to a [2-[2-[2-maleimidopropionamido(ethoxy)ethoxy]acetic acid linker, which is turn covalently linked to the epsilon amino of the carboxy terminal lysine, i.e., lysine 40, of exendin-4(1-39) Lys⁴⁰-NH₂.

In some embodiments, the pharmaceutical formulation comprises about 1 mg/ml to about 15 mg/ml exendin-4(1-39) Lys⁴⁰ (ε-AEEA-MPA)-NH₂ albumin conjugate in 5-30 mM sodium phosphate buffer at pH 6.5-7.5 containing 100-200 mM sodium chloride, 1-10 mM sodium octanoate, and 1-30 mg/L polysorbate 80. In a particular embodiment, the formulation comprises 10 mg/ml exendin-4(1-39) Lys⁴⁰ (ε-AEEA-MPA)-NH₂ albumin conjugate in 5-30 mM sodium phosphate buffer at pH 6.5-7.5 containing 100-200 mM sodium chloride, 1-10 mM sodium octanoate, and 1-30 mg/L polysorbate 80. In a particular embodiment, the formulation comprises 10 mg/ml exendin-4(1-39) Lys⁴⁰ (ε-AEEA-MPA)-NH₂ albumin conjugate in 10 mM sodium phosphate buffer containing 100-200 mM sodium chloride, 1-10 mM sodium octanoate, and 1-30 mg/L polysorbate 80 wherein said formulation has a pH of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. In a particular embodiment, the formulation comprises 10 mg/ml exendin-4(1-39) Lys⁴⁰ (ε-AEEA-MPA)-NH₂ albumin conjugate in 10 mM sodium phosphate buffer at pH 7.0 containing 100-200 mM sodium chloride, 1-10 mM sodium octanoate, and 1-30 mg/L polysorbate 80. In a particular embodiment, the formulation comprises 10 mg/ml exendin-4(1-39) Lys⁴⁰ (ε-AEEA-MPA)-NH₂ albumin conjugate in 10 mM sodium phosphate buffer at pH 7.0 containing 135 mM sodium chloride, 1.6 mM sodium octanoate, and 15 mg/L polysorbate 80. In a particular embodiment, the formulation consists of about 1 mg/ml to about 15 mg/ml exendin-4(1-39) Lys⁴⁰ (ε-AEEA-MPA)-NH₂ albumin conjugate in 10 mM sodium phosphate buffer at pH 7.0 containing 135 mM sodium chloride, 1.6 mM sodium octanoate, and 15 mg/L polysorbate 80. In a particular embodiment, the formulation consists of 10 mg/ml exendin-4(1-39) Lys⁴⁰ (ε-AEEA-MPA)-NH₂ albumin conjugate in 10 mM sodium phosphate buffer at pH 7.0 containing 135 mM sodium chloride, 1.6 mM sodium octanoate, and 15 mg/L polysorbate 80.

In some embodiments, the pharmaceutical formulation comprises about 1 mg/ml to about 15 mg/ml exendin-4(1-39) Lys⁴⁰ (ε-AEEA-MPA)-NH₂ albumin conjugate in 5-30 mM sodium acetate buffer at pH 4.5-5.5, containing 1-15 mM sodium octanoate, 0.05 to 0.2% (w/v) pluronic F68, and either 100-200 mM sodium chloride or 2-8% (w/v) sorbitol. In a particular embodiment, the formulation comprises 10 mg/ml exendin-4(1-39) Lys⁴⁰ (ε-AEEA-MPA)-NH₂ albumin conjugate in 5-30 mM sodium acetate buffer at pH 4.5-5.5, containing 1-15 mM sodium octanoate, 0.05 to 0.2% (w/v) pluronic F68, and either 100-200 mM sodium chloride or 2-8% (w/v) sorbitol. In a particular embodiment, the formulation comprises 10 mg/ml exendin-4(1-39) Lys⁴⁰ (ε-AEEA-MPA)-NH₂ albumin conjugate in 10 mM sodium acetate buffer containing 1-15 mM sodium octanoate, 0.05 to 0.2% (w/v) pluronic F68, and either 100-200 mM sodium chloride or 2-8% (w/v) sorbitol wherein said formulation has a pH of about 4.5, 4.6, 4.7, 4.8, 4.9, 5.0, 5.1, 5.2, 5.3, 5.4, or 5.5. In a particular embodiment, the formulation comprises 10 mg/ml exendin-4(1-39) Lys⁴⁰ (ε-AEEA-MPA)-NH₂ albumin conjugate in 10 mM sodium acetate buffer at pH 5.0 containing 1-15 mM sodium octanoate, 0.05 to 0.2% (w/v) pluronic F68, and either 100-200 mM sodium chloride or 2-8% (w/v) sorbitol. In a particular embodiment, the formulation comprises 10 mg/ml exendin-4(1-39) Lys⁴⁰ (ε-AEEA-MPA)-NH₂ albumin conjugate in 10 mM sodium acetate buffer at pH 5.0 containing 150 mM sodium chloride, 5 mM sodium octanoate and 0.1% (w/v) pluronic F68 (i.e., poloxamer 188). In a particular embodiment, the formulation consists of about 1 mg/ml to about 15 mg/ml exendin-4(1-39) Lys⁴⁰ (ε-AEEA-MPA)-NH₂ albumin conjugate in 10 mM sodium acetate buffer at pH 5.0 containing 150 mM sodium chloride, 5 mM sodium octanoate and 0.1% (w/v) pluronic F68 (i.e., poloxamer 188). In a particular embodiment, the formulation consists of 10 mg/ml exendin-4(1-39) Lys⁴⁰ (ε-AEEA-MPA)-NH₂ albumin conjugate in 10 mM sodium acetate buffer at pH 5.0 containing 150 mM sodium chloride, 5 mM sodium octanoate and 0.1% (w/v) pluronic F68 (i.e., poloxamer 188).

In another aspect, the present invention provides methods for treating diabetes, obesity or other diseases or conditions treatable with an insulinotropic peptide, such as pre-diabetes (e.g., impaired glucose tolerance (IGT) or impaired fasting glucose (IFG)), diabetes, e.g., type I diabetes, type II diabetes, late autoimmune diabetes in adults (“LADA”) also known as late onset autoimmune diabetes of adulthood, slow onset type I diabetes, type 1.5 diabetes, steroid induced diabetes, Human Immunodeficiency Virus (HIV) Treatment-Induced Diabetes, diabetes development in subjects with congenital or HIV-Associated Lipodystrophy (“Fat Redistribution Syndrome”), obesity (i.e., BMI of 30 kg/m² or greater), overweight (i.e., BMI between 25 kg/m² and 30 kg/m²), metabolic syndrome (Syndrome X), nervous system disorders, surgery, insulin resistance, hypoglycemia unawareness, restrictive lung disease, gastrointestinal disorders, e.g., irritable bowel syndrome (IBS), functional dyspepsia, pain associated with gastrointestinal disorders, e.g., pain associated with IBS and functional dyspepsia, inflammatory bowel disease (IBD), e.g., Crohn's disease, ulcerative colitis, pain associated with IBD, hyperglycemia, e.g., hyperglycemia associated with surgery (e.g., a major surgical procedure, e.g., coronary bypass surgery) e.g., hyperglycemia associated with surgery on subjects with diabetes, e.g., type II diabetes, metabolic syndrome, coronary heart failure (CHF), disorders associated with beta cell disfunction, disorders associated with the absence of beta cells, disorders associated with insufficient numbers of beta cells, or other conditions treatable with an insulinotropic peptide or insulinotropic peptide conjugate, comprising administering to a subject the insulinotropic peptide conjugate, e.g., in a pharmaceutical formulation described herein.

In another aspect, the present invention provides methods for treating diabetes, obesity, or other disorders treatable with an insulinotropic peptide by administering to a subject an effective amount of an insulinotropic peptide conjugate, e.g., in a pharmaceutical formulation described herein in combination with one or more second therapeutic agents. In some embodiments, the second therapeutic agent is an anti-diabetic agent. In some embodiments, the anti-diabetic agent is an oral antidiabetic agent (OAD), e.g., a biguanide, e.g., metformin.

The invention also encompasses kits comprising pharmaceutical formulations and dosage forms of the invention.

### 4. BRIEF DESCRIPTION OF THE FIGURES

FIG. 1 presents a graph representing an SEC-HPLC time course purity plot of formulations incubated at 6 months at 25° C.

FIG. 2 presents a graph representing an SEC-HPLC time course purity plot of formulations incubated at 3 months at 40° C.

FIG. 3 presents a graph representing an RP-HPLC peptide degradant plot of formulations incubated at 6 months at 25° C.

FIG. 4 presents a graph representing an RP-HPLC peptide degradant plot of formulations incubated at 3 months at 40° C.

FIG. 5 presents a graph representing an SEC-HPLC purity comparison of formulations containing sodium acetate v. sodium phosphate buffers at 25° C.

FIG. 6 presents a graph representing an RP-HPLC peptide degradant comparison of formulations containing sodium acetate v. sodium phosphate buffers at 25° C.

FIG. 7 presents an SDS-PAGE comparison of formulations containing sodium acetate v. sodium phosphate buffers after six months at 25° C.

FIG. 8 presents a graph representing an SEC-HPLC purity comparison of formulations with various pH at 25° C.

FIG. 9 presents a graph representing an RP-HPLC peptide degradant comparison of formulations with various pH at 25° C.

FIG. 10 presents a graph representing an SEC-HPLC purity comparison of pH 5.0 formulations containing various tonicity modifiers at 25° C.

FIG. 11 presents a graph representing an RP-HPLC peptide degradant comparison of pH 5.0 formulations containing various tonicity modifiers at 25° C.

FIG. 12 presents a graph representing an SEC-HPLC purity comparison of pH 6.0 formulations containing various stabilizers at 25° C.

FIG. 13 presents a graph representing an RP-HPLC peptide degradant comparison of pH 6.0 formulations containing various stabilizers at 25° C.

FIG. 14 presents a graph representing an SEC-HPLC purity comparison of pH 6, sorbitol formulations containing various concentration of exendin-4(1-39) Lys⁴⁰ (ε-AEEA-MPA)-NH₂ albumin conjugate at 25° C.

FIG. 15 presents a graph representing an RP-HPLC purity comparison of pH 6, sorbitol formulations containing various concentration of exendin-4(1-39) Lys⁴⁰ (ε-AEEA-MPA)-NH₂ albumin conjugate at 25° C.

FIG. 16 presents a graph representing an SEC-HPLC purity plot of formulations containing 10 mg/ml exendin-4(1-39) Lys⁴⁰ (ε-AEEA-MPA)-NH₂ albumin conjugate, sodium acetate buffer of pH 5.0, 150 mM sodium chloride and 5 mM sodium octanoate.

FIG. 17 presents a graph representing an SEC-HPLC purity plot of formulations containing 10 mg/ml exendin-4(1-39) Lys⁴⁰ (ε-AEEA-MPA)-NH₂ albumin conjugate, sodium phosphate buffer of pH 5.0, 150 mM sodium chloride and 5 mM sodium octanoate.

FIG. 18 presents a graph representing an RP-HPLC peptide degradant plot of formulations containing 10 mg/ml exendin-4(1-39) Lys⁴⁰ (ε-AEEA-MPA)-NH₂ albumin conjugate, sodium acetate buffer of pH 5.0, 150 mM sodium chloride and 5 mM sodium octanoate.

FIG. 19 presents a graph representing an RP-HPLC peptide degradant plot of formulations containing 10 mg/ml exendin-4(1-39) Lys⁴⁰ (ε-AEEA-MPA)-NH₂ albumin conjugate, sodium phosphate buffer of pH 5.0, 150 mM sodium chloride and 5 mM sodium octanoate.

### 5. DETAILED DESCRIPTION OF THE PREFERRED EMBODIMENTS

### 5.1 Definitions

As used herein, the following terms shall have the following meanings unless otherwise specified:

As used herein, “about” refers to a value that is no more than 10% above or below the value being modified by the term, unless otherwise indicated. For example, the term “about 20 mg/ml” means a range of from 18 mg/ml to 22 mg/ml. Where “about” is used with respect to a pH range, for instance, “about pH 5.0,” the pH value is no more than 0.5 above or below the pH being modified by the term. Thus, “about pH 5.0” means a range of from pH 4.5 to 5.5. Similarly “about pH 7.0” means a range of from pH 6.5 to pH 7.5.

As used herein, “subject” refers to an animal such as a mammal, including but not limited to, a primate (e.g., human), cow, sheep, goat, horse, dog, cat, rabbit, rat, mouse and the like. In preferred embodiments, the subject is human. In certain embodiments, the subject is a non-human animal, for instance, a non-human animal such as a cow, sheep, goat or horse. The subject can be male or female.

As used herein, “insulinotropic” means having insulinotropic activity, i.e., the ability to stimulate, or to cause the stimulation of, the synthesis or expression of the hormone insulin. Insulinotropic peptides include, but are not limited to, GLP-1, exendin-3, exendin-4, and precursors, derivatives, or fragments of peptides such as GLP-1, exendin-3 and exendin-4 and other peptides with insulinotropic activity.

“Glucagon-Like Peptide-1” (“GLP-1”) and “GLP-1 derivatives” are intestinal hormones which generally simulate insulin secretion during hyperglycemia, suppress glucagon secretion, stimulate (pro) insulin biosynthesis and decelerate gastric emptying and acid secretion. In some embodiments, the glucagon-like peptide is GLP-1(7-37). In some embodiments, the glucagon-like peptide is GLP-1(7-36). Some GLPs and GLP derivatives, such as those described herein as SEQ ID NOS: 3-15, promote glucose uptake by cells but do not simulate insulin expression, as disclosed in U.S. Pat. No. 5,574,008, which is incorporated by reference herein in its entirety.

“Exendin-3” is a naturally occurring GLP-1 agonist isolated from salivary secretions of Heloderma horridum, the Mexican bearded lizard, and shares a 53% overlap with mammalian GLP-1 amino acid sequence, as disclosed in U.S. Pat. No. 5,424,286, which is incorporated by reference herein in its entirety. The amino acid sequence of exendin-3 is HSDGTFTSDLSKQMEEEAVRLFIEWLKNGGPSSGAPPPS (SEQ ID NO:16).

“Exendin-4” is a naturally occurring GLP-1 agonist isolated from salivary gland venom of Heloderma suspectum, the Gila monster, and shares a 53% overlap with mammalian GLP-1 amino acid sequence as disclosed in U.S. Pat. No. 5,424,286, which is incorporated by reference herein in its entirety. The amino acid sequence of exendin-4 is HGEGTFTSDLSKQMEEEAVRLFIEWLKNGGPSSGAPPPS (SEQ ID NO:17). Exendin-4 decreases glucagons and increases insulin secretion in a glucose-dependent manner, and mimics certain actions of GLP-1, including binding to and activating the human GLP-1 receptor. Exendin-4 improves glycemic control by reducing fasting and postprandial glucose concentrations through restoration of first-phase insulin response, regulation of glucagon secretion, delaying gastric emptying, and decreasing food intake.

“Reactive groups” are chemical groups capable of forming a covalent bond. Such reactive agents can be coupled or bonded to an insulinotropic peptide of interest to form a modified insulinotropic peptide. Reactive groups can generally be carboxy, phosphoryl, or acyl groups, either as an ester or a mixed anhydride, or an imidate, thereby capable of forming a covalent bond with functionalities such as an amino group, a hydroxy or a thiol at the target site on albumin. For the most part, the esters will involve phenolic compounds, or be thiol esters, alkyl esters, phosphate esters, or the like. Reactive groups include succinimidyl and maleimido groups.

“Functionalities” are groups on albumin to which reactive groups on modified insulinotropic peptides are capable of reacting with to form covalent bonds. Functionalities include hydroxyl groups for bonding to ester reactive entities; thiol groups for bonding to maleimides and maleimido groups, imidates and thioester groups; and amino groups for bonding to carboxy, phosphoryl or acyl groups on reactive entities.

“Linking Groups” are chemical moieties that can be used to connect reactive groups to insulinotropic peptides. Linking groups can comprise one or more alkyl groups such as methyl, ethyl, propyl, butyl, etc. groups, alkoxy groups, alkenyl groups, alkynyl groups or amino group substituted by alkyl groups, cycloalkyl groups, polycyclic groups, aryl groups, polyaryl groups, substituted aryl groups, heterocyclic groups, and substituted heterocyclic groups. Linking groups can also comprise poly ethoxy aminoacids such as AEA ((2-amino) ethoxy acetic acid) or a preferred linking group AEEA ([2-(2-amino)ethoxy)]ethoxy acetic acid).

As used herein, “albumin” refers to the most abundant protein in blood plasma having a molecular weight of approximately between 65 and 67 kilodaltons in its monomeric form, depending on the species of origin. The term “albumin” is used interchangeably with “serum albumin” and is not meant to define the source of the albumin which forms a conjugate with the insulinotropic peptides of the invention. Thus, the term “albumin” as used herein can refer either to albumin purified from a natural source such as blood or serous fluids, or it can refer to chemically synthesized albumin, or albumin produced by recombinant techniques. Exemplary forms of albumin of the insulinotropic peptide conjugates described herein are provided in section 5.5.5.1 below.

An “insulinotropic peptide conjugate” comprises an insulinotropic peptide that has been conjugated to albumin via a covalent bond formed between the insulinotropic peptide and a functionality on albumin. In some embodiments, the insulinotropic peptide has been modified to contain a reactive group to which albumin is covalently bound. In some embodiments, the reactive group is coupled to the insulinotropic peptide via a linking group.

“Stable” formulations include formulations in which the peptide or peptide conjugate therein essentially retains its physical stability and/or chemical stability and/or biological activity upon storage. Various analytical techniques for measuring protein stability are available in the art and are reviewed in Lee, V., 1991, Peptide and Protein Drug Delivery, 247-301 (Marcel Dekker, Inc., New York, N.Y.) and Jones, A. 1993, Adv. Drug Delivery Rev. 10: 29-90, for example. Stability can be measured at a selected temperature for a selected time period. Preferably, the formulation is stable at room temperature (about 25° C.) or at 40° C. for at least 1, 2, 3, 4, 5 or 6 months and/or stable at about 2-8° C. for at least 1, 2, 3, 4, 5 or 6 months. Furthermore, in certain embodiments, the formulation is preferably stable following freezing (e.g., −70° C.). In certain embodiments, the criteria for stability are as follows: (1) the formulation remains clear by visual analysis; (2) the concentration, pH and osmolality of the formulation has no more than about ±10% change; (3) no more than about 10%, more preferably no more than about 5%, or most preferably no more than about 1% of aggregate forms as measured by SEC-HPLC; and (4) no more than 10%, more preferably no more than about 5%, or most preferably no more than 1% of peptide or peptide conjugate breaks down as measured by SDS-PAGE or RP-HPLC.

As used herein, a “stabilizer” is that which achieves a “stable” formulation as defined herein.

A peptide or peptide conjugate “retains its physical stability” in a pharmaceutical formulation if it shows substantially no signs of aggregation, precipitation and/or denaturation upon visual examination of color and/or clarity, or as measured by UV light scattering or by size exclusion chromatography. For example, the peptide of a peptide-conjugate retains its physical stability in a pharmaceutical formulation where less than about 10%, more preferably less than about 5, or most preferably less than about 1% of the peptide or peptide conjugate is present as an aggregate in the formulation.

A peptide or peptide conjugate “retains its chemical stability” in a pharmaceutical formulation if the chemical stability at a given time is such that the peptide is considered to retain its biological activity as defined below. Chemical stability can be assessed by detecting and quantifying chemically altered forms of the peptide. Chemical alteration may involve size modification (e.g. clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g. occurring as a result of deamidation) which can be evaluated by ion-exchange chromatography, for example.

A peptide or peptide conjugate “retains its biological activity” in a pharmaceutical formulation, if the peptide in a pharmaceutical formulation is biologically active for its intended purpose. For example, biological activity is retained if the biological activity of the peptide in the pharmaceutical formulation is at least about 70%, at least about 80%, or more preferably, at least about 90% (within the errors of the assay) of the biological activity exhibited at the time the pharmaceutical formulation was prepared. The biological activity for a particular peptide will be the biological activity of the peptide known to those of skill in the art. For example, the biological activity of GLP-1 includes, but is not limited to, stimulation of insulin secretion during hyperglycemia, suppression of glucagon secretion, stimulation of (pro) insulin biosynthesis, deceleration of gastric emptying and acid secretion, and reduction of blood glucose levels.

As used herein, a “buffer” refers to a buffered solution that resists changes in pH and maintains the pH value of a solution in an acceptable range by the action of its acid-base conjugate components. The buffer of this invention has a pH in the range from about 4 to about 8; preferably from about 5 to about 7; and most preferably has a pH in the range from about 5 to about 6. In some embodiments, the pH of the buffer is about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9 or 8.0. Examples of buffers that will control the pH in this range include acetate (e.g. sodium acetate), phosphate (e.g. sodium phosphate), succinate (such as sodium succinate), gluconate, histidine, citrate and other organic acid buffers.

As used herein, a “tonicity modifier” refers to a compound which, in appropriate amount, renders the formulation isotonic, including, for example, sodium chloride, calcium chloride, magnesium chloride, lactose, sorbitol, sucrose, mannitol, trehalose, raffinose, polyethylene glycol, hydroxyethyl starch, glycine and the like. “Isotonic” is meant that the formulation of interest has essentially the same osmolarity as human blood. Isotonic formulations will generally have an osmolarity from about 250 to 350 mOsm, preferably from about 250 to about 330 mOsm. Osmolarity can be measured using a vapor pressure or ice-freezing type osmometer, for example.

As used herein, a “surfactant” refers to a compound that reduces interfacial tension between a liquid and a solid when dissolved in solution, which can be added to the formulation to reduce aggregation of the reconstituted protein and/or reduce the formation of particulates in the reconstituted formulation. Examples of surfactants useful for the formulations and methods described herein include polysorbates (e.g. polysorbates 20 or 80); poloxamers (e.g. poloxamer 188 (pluronic F68)); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethyl glycol, polypropyl glycol, and copolymers of ethylene and propylene glycol, etc.

As used herein, a “preservative” refers to a compound which can be added to the formulation to essentially reduce bacterial activity therein, thus facilitating the production of a multi-use formulation, for example. Examples of potential preservatives include m-cresol, benzyl alcohol, methanol, ethanol, iso-propanol, butyl paraben, ethyl paraben, methyl paraben, phenol, glycerol, xylitol, resorcinol, cathechol, 2,6-dimethylcyclohexanol, 2-methyl-2,4-pentadiol, dextran, polyvinylpyrrolidone, 2-chlorophenol, benzethonium chloride, merthiolate (thimersosal), benzoic acid (propyl paraben) MW 180.2, benzoic acid MW 122.12, benzalkonium chloride, chlorobutanol, sodium benzoate, sodium propionate, and cetylpyridinium chloride.

As used herein, a “bulking agent” refers to a compound which can add mass to a lyophilized mixture and contributes to the physical structure of a lyophilized cake (e.g. facilitates the production of an essentially uniform lyophilized cake which maintains an open pore structure). Exemplary bulking agents include mannitol, glycine, polyethylene glycol and xorbitol. In addition to providing a pharmaceutically acceptable cake, bulking agents also typically impart useful qualities to the lyophilized composition such as modifying the collapse temperature, providing freeze-thaw protection, further enhancing the protein stability over long-term storage, and the like. These agents can also serve as tonicity modifiers.

As used herein, a “reducing sugar” is one which contains a hemiacetal group that can reduce metal ions or react covalently with lysine and other amino groups in proteins and a “non-reducing sugar” is one which does not have these properties of a reducing sugar. Examples of reducing sugars are fructose, mannose, maltose, lactose, arabinose, xylose, ribose, rhamnose, galactose and glucose. Nonreducing sugars include sucrose, trehalose, sorbose, melezitose and raffinose. Preferably, lyophilized pharmaceutical formulations as described herein are lyophilized in the absence of reducing sugars, or in the presence of only non-reducing sugars.

As used herein, a “pharmaceutically acceptable carrier” refers to a pharmaceutically acceptable material, composition or vehicle, suitable for administration to mammals, preferably humans. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not overly injurious (e.g., fatal) to the subject. In a preferred embodiment, the pharmaceutically acceptable carrier is approved for administration to humans by a government regulatory agency such as the Food and Drug Administration (FDA) or the European Medicines Agency (EMEA).

“Preventing” or “prevention” of any disease or disorder refers to a reduction in the risk of acquiring a disease or disorder (i.e., causing at least one of the clinical symptoms of the disease not to develop in a subject that may be exposed or predisposed to the disease but does not yet experience or display symptoms of the disease). Preferably, prevention refers to the use of a compound or composition in a subject not yet affected by the disease or disorder or not yet exhibiting a symptom of the disease or disorder, for instance a subject not yet diabetic or not yet exhibiting the symptoms of diabetes.

“Treating” or “treatment” of any disease or disorder refers, in one embodiment, to ameliorating the disease or disorder (i.e., arresting or reducing the development of the disease or at least one of the clinical symptoms thereof) that exists in a subject. In another embodiment, “treating” or “treatment” refers to ameliorating at least one physical parameter, which may be indiscernible by the subject. In yet another embodiment, “treating or treatment” refers to modulating the disease, either physically (e.g., stabilization of a discernable symptom) or physiologically (e.g., stabilization of a physical parameter) or both.

As used herein, an “effective amount,” with respect to treatment, means an amount of an insulinotropic peptide conjugate that when, administered to a subject for treating a disease is sufficient to treat the disease. An effective amount can vary depending on, inter alia, the insulinotropic peptide used, the disease and its severity and the age, weight, etc. of the subject to be treated.

### 5.2 Pharmaceutical Formulation

The present invention provides pharmaceutical formulations of insulinotropic peptide conjugates. The formulations can be suitable for administration via a parenteral route such as subcutaneous, intravenous, intramuscular, transdermal, intra-arterial, or intra-peritoneal routes, or via other routes such as oral, topical, or inhalation routes.

The insulinotropic peptide in the conjugate can be any insulinotropic peptide known to those of skill in the art. It can be any peptide that is capable of stimulating, or causing the stimulation of, synthesis or expression of the hormone insulin. In some embodiments, the insulinotropic peptide is selected from the group consisting of glucagon-like peptide 1, exendin-3 and exendin-4 and their precursors, derivatives or fragments. In certain embodiments, the insulinotropic peptide is exendin-4 or a derivative. Exemplary derivatives are described in detail below.

In some embodiments, the insulinotropic peptide is conjugated to albumin. In some embodiments, the insulinotropic peptide is conjugated to serum albumin. In some embodiments, the insulinotropic peptide is conjugated to human serum albumin. In some embodiments, the insulinotropic peptide is conjugated to recombinant human serum albumin. The insulinotropic peptide and insulinotropic peptide conjugate are described in detail in Section 5.5 below.

It is contemplated that free albumin may be present in the formulations, at a concentration of about 80, 70, 60, 50, 40, 30, 25, 20, 15, 14, 13, 12, 11, 10, 9, 8, 7, 6, 5, 4, 3, 2, 1, 0.5, 0.1, 0.05 or 0.01 mg/ml. In certain embodiments, free albumin is present at less than about 80, 70, 60, 50, 40, 30, 20, 25, 15, 14, 13, 12, 11, 10, 9, 8, 7, 6, 5, 4, 3, 2, 1, 0.5, 0.1, 0.05 or 0.01 mg/ml. Preferably, free albumin is present at less than or equal to about 15 mg/ml, more preferably free albumin is present at less than or equal to 10 mg/ml, and most preferably less than 5 mg/ml. In some embodiments, the free albumin present in the formulations described herein is less than or equal to 10 mg/ml. In some embodiments, the free albumin present in the formulations described herein is less than or equal to 1 mg/ml. In some embodiments, the free albumin present in the formulations described herein is less than or equal to 0.5 mg/ml. In some embodiments, the free albumin present in the formulations described herein is less than or equal to 0.1 mg/ml. In some embodiments, the free albumin present in the formulations described herein is less than or equal to 0.05 mg/ml.

Actual dosage levels of insulinotropic peptide conjugates in the formulations of the present invention can be varied so as to obtain an amount of the active ingredient which is effective to achieve the desired therapeutic response for a particular subject, composition, and mode of administration, without being toxic to the subject. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the subject being treated, and like factors well known in the medical arts.

In certain embodiments, the formulations according to the present invention are suitable for subcutaneous administration of an insulinotropic peptide conjugate to a subject in need thereof. In some embodiments, the subject is administered a dose of the insulinotropic peptide conjugate in an amount between about 1000 μg and 3000 μg (e.g., 1025 μg, 1050 μg, 1075 μg, 1100 μg, 1125 μg, 1150 μg, 1175 μg, 1200 μg, 1225 μg, 1250 μg, 1275 μg, 1300 μg, 1325 μg, 1350 μg, 1375 μg, 1400 μg, 1425 μg, 1450 μg, 1475 μg, 1500 μg, 1525 μg, 1550 μg, 1575 μg, 1600 μg, 1625 μg, 1650 μg, 1675 μg, 1700 μg, 1725 μg, 1750 μg, 1775 μg, 1800 μg, 1825 μg, 1850 μg, 1875 μg, 1900 μg, 1925 μg, 1950 μg, 1975 μg, 2000 μg, 2025 μg, 2050 μg, 2075 μg, 2100 μg, 2125 μg, 2150 μg, 2175 μg, 2200 μg, 2225 μg, 2250 μg, 2275 μg, 2300 μg, 2325 μg, 2350 μg, 2375 μg, 2400 μg, 2425 μg, 2450 μg, 2475 μg, 2500 μg, 2525 μg, 2550 μg, 2575 μg, 2600 μg, 2625 μg, 2650 μg, 2675 μg, 2700 μg, 2725 μg, 2750 μg, 2775 μg, 2800 μg, 2825 μg, 2850 μg, 2875 μg, 2900 μg, 2925 μg, 2950 μg, or 2975 μg), preferably between about 1000 μg and 2750 μg (e.g., 1025 μg, 1050 μg, 1075 μg, 1100 μg, 1125 μg, 1500 μg, 1175 μg, 1200 μg, 1225 μg, 1250 μg, 1275 μg, 1300 μg, 1325 μg, 1350 μg, 1375 μg, 1400 μg, 1425 μg, 1450 μg, 1475 μg, 1500 μg, 1525 μg, 1550 μg, 1575 μg, 1600 μg, 1625 μg, 1650 μg, 1675 μg, 1700 μg, 1725 μg, 1750 μg, 1775 μg, 1800 μg, 1825 μg, 1850 μg, 1875 μg, 1900 μg, 1925 μg, 1950 μg, 1975 μg, 2000 μg, 2025 μg, 2050 μg, 2075 μg, 2100 μg, 2125 μg, 2150 μg, 2175 μg, 2200 μg, 2225 μg, 2250 μg, 2275 μg, 2300 μg, 2325 μg, 2350 μg, 2375 μg, 2400 μg, 2425 μg, 2450 μg, 2475 μg, 2500 μg, 2525 μg, 2550 μg, 2575 μg, 2600 μg, 2625 μg, 2650 μg, 2675 μg, 2700 μg, or 2725 μg), and more preferably between about 1000 and 2500 μg (e.g., 1025 μg, 1050 μg, 1075 μg, 1100 μg, 1125 μg, 1150 μg, 1175 μg, 1200 μg, 1225 μg, 1250 μg, 1275 μg, 1300 μg, 1325 μg, 1350 μg, 1375 μg, 1400 μg, 1425 μg, 1450 μg, 1475 μg, 1500 μg, 1525 μg, 1550 μg, 1575 μg, 1600 μg, 1625 μg, 1650 μg, 1675 μg, 1700 μg, 1725 μg, 1750 μg, 1775 μg, 1800 μg, 1825 μg, 1850 μg, 1875 μg, 1900 μg, 1925 μg, 1950 μg, 1975 μg, 2000 μg, 2025 μg, 2050 μg, 2075 μg, 2100 μg, 2125 μg, 2150 μg, 2175 μg, 2200 μg, 2225 μg, 2250 μg, 2275 μg, 2300 μg, 2325 μg, 2350 μg, 2375 μg, 2400 μg, 2425 μg, 2450 μg, or 2475 μg), most preferably between about 1000 μg to 2000 μg (e.g., 1025 μg, 1050 μg, 1075 μg, 1100 μg, 1125 μg, 1150 μg, 1175 μg, 1200 μg, 1225 μg, 1250 μg, 1275 μg, 1300 μg, 1325 μg, 1350 μg, 1375 μg, 1400 μg, 1425 μg, 1450 μg, 1475 μg, 1500 μg, 1525 μg, 1550 μg, 1575 μg, 1600 μg, 1625 μg, 1650 μg, 1675 μg, 1700 μg, 1725 μg, 1750 μg, 1775 μg, 1800 μg, 1825 μg, 1850 μg, 1875 μg, 1900 μg, 1925 μg, 1950 μg, or 1975 μg) of the insulinotropic peptide conjugate.

In some embodiments, the dosage of insulinotropic peptide conjugate, e.g., insulinotropic peptide conjugate formulation, which may be effective to treat a disease or condition described herein for a particular subject is administered to the subject in accordance with a weekly dosing regime. Thus, in certain embodiments, the subject can be administered a total weekly dosage of the insulinotropic peptide conjugate over a number of weeks to achieve the desired therapeutic response. In certain embodiments, the total weekly dose is administered in a single administration during the week, i.e., once a week, and the total weekly dose comprises the insulinotropic peptide conjugate in an amount of 1000 μg or 1500 μg. In certain embodiments, the total weekly dose is administered once a week, and the dose comprises the insulinotropic peptide conjugate in an amount of 2000 μg.

In certain embodiments, the total weekly dose is administered over two administrations during the week, i.e., twice a week, and each administration comprises the insulinotropic peptide conjugate in an amount of 1000 μg, amounting to a total weekly dose of 2000 μg. In certain embodiments, the total weekly dose is administered twice a week, and each administration comprises the insulinotropic peptide conjugate in an amount of 1500 μg, amounting to a total weekly dose of 3000 μg. In certain embodiments, the total weekly dose is administered twice a week, and each administration comprises the insulinotropic peptide conjugate in an amount of 1600 μg, amounting to a total weekly dose of 3200 μg. In certain embodiments, the total weekly dose is administered twice a week, and each administration comprises the insulinotropic peptide conjugate in an amount of 1700 μg, amounting to a total weekly dose of 3400 μg. In certain embodiments, the total weekly dose is administered twice a week, wherein the first administration comprises the insulinotropic peptide conjugate in an amount of 1500 μg and the second administration comprises the insulinotropic peptide in an amount of 2000 μg, amounting to a total weekly dose of 3500 μg. In certain embodiments, the total weekly dose is administered twice a week, and each administration comprises the insulinotropic peptide conjugate in an amount of 1750 μg, amounting to a total weekly dose of 3500 μg. In certain embodiments, the total weekly dose is administered twice a week, and each administration comprises the insulinotropic peptide conjugate in an amount of 1800 μg, amounting to a total weekly dose of 3600 μg. In certain embodiments, the total weekly dose is administered twice a week, and each administration comprises the insulinotropic peptide conjugate in an amount of 1900 μg, amounting to a total weekly dose of 3800 μg. In certain embodiments, the total weekly dose is administered twice a week, and each administration comprises the insulinotropic peptide conjugate in an amount of 2000 μg, amounting to a total weekly dose of 4000 μg.

In other embodiments, the insulinotropic peptide conjugate, e.g., insulinotropic peptide conjugate formulation, can be administered once every 8, 9, 10, 11, 12 or 13 days. In other embodiments, the insulinotropic peptide conjugate, e.g., insulinotropic peptide conjugate formulation, can be administered two times every 3, 4, 5, 6, 7 or 8 day period. In other embodiments, the insulinotropic peptide conjugate, e.g., insulinotropic peptide conjugate formulation, can be administered two times every 9, 10, 11, 12, 13 or 14 day period.

In some embodiments, the concentration of the insulinotropic peptide conjugate (without free albumin) in the formulations is from about 0.1 mg/ml to about 100 mg/ml, from about 0.1 mg/ml to about 75 mg/ml, from about 0.1 mg/ml to about 50 mg/ml, from about 0.1 mg/ml to about 40 mg/ml, from about 0.1 mg/ml to about 30 mg/ml, from about 1 mg/ml to about 100 mg/ml, from about 5 mg/ml to about 50 mg/ml, or from about 10 mg/ml to 20 mg/ml. In some embodiments, the concentration of the insulinotropic peptide conjugate in the formulations is higher than about 10 mg/ml, about 20 mg/ml, about 50 mg/ml, about 100 mg/ml, about 200 mg/ml, or about 500 mg/ml. In some embodiments, the concentration of the insulinotropic peptide conjugate in the formulations is lower than about 100 mg/ml, about 50 mg/ml, about 40 mg/ml, about 30 mg/ml, about 20 mg/ml, about 10 mg/ml, about 5 mg/ml, about 1 mg/ml, or about 0.1 mg/ml. In preferred embodiments, the concentration of the insulinotropic peptide conjugate in the formulations is about 1 mg/ml to about 50 mg/ml, from about 1 mg/ml to about 40 mg/ml, from about 1 mg/ml to about 20 mg/ml, or from about 1 to about 15 mg/ml. In particularly preferred embodiments, the concentration of the insulinotropic peptide conjugate in the formulations is about 1 mg/ml. In other particularly preferred embodiments, the concentration of the insulinotropic peptide conjugate in the formulations is about 2.5 mg/ml. In other particularly preferred embodiments, the concentration of the insulinotropic peptide conjugate in the formulations is about 5 mg/ml. In other particularly preferred embodiments, the concentration of the insulinotropic peptide conjugate in the formulations is about 10 mg/ml.

In certain embodiments, the formulations herein can be administered as monotherapy. In other words, the formulations herein can be provided as the sole administration of an active agent for treatment of one or more conditions provided herein.

The formulations herein can also be administered in combination with or can comprise one or more second therapeutic agents useful for the particular indication being treated, preferably those with complementary activities that do not adversely affect the insulinotropic peptide conjugate of the formulation. In certain embodiments, such second therapeutic agents can be present with the insulinotropic peptide conjugate in amounts that are effective for the purpose intended. In a particular embodiment, the second therapeutic agent is an anti-diabetic agent, e.g., an oral anti-diabetic agent, e.g., a biguanide, e.g., metformin.

The pharmaceutical formulations can comprise a buffer that maintains a physiologically suitable pH. In addition, the buffer can serve to enhance isotonicity and chemical stability of the formulation. In some embodiments, the formulation has a pH of about 3.0 to 8.0. In some embodiments, the formulation has a pH of about 4.0 to 8.0. In some embodiments, the formulation has a pH of about 4.0 to 6.0. In some embodiments, the formulation has a pH of about 6.0 to 8.0. In some embodiments, the formulation has a pH of about 6.0 to 9.0. In some embodiments, the formulation has a pH of about 5.0 to 7.0. In some embodiments, the formulation has a pH of about 4.5 to 6.0. In some embodiments, the formulation has a pH of about 5.0 to 6.0. In some embodiments, the formulation has a pH of about 5.1 to 6.0, about 5.2 to 6.0, about 5.3 to 6.0, about 5.4 to 6.0, about 5.5 to 6.0, about 5.6 to 6.0, about 5.7 to 6.0, or about 5.8 to 6.0. In some embodiments, said formulation has a pH of about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9 or 9.0. In a particular embodiment, the formulation has a pH of about 5.0. In another particular embodiment, the formulation has a pH of about 7.0. The pH can be adjusted as necessary by techniques known in the art. For example, hydrochloric acid or sodium hydroxide can be added as necessary to adjust the pH to desired levels.

Useful buffers in the formulations of the present invention include, but are not limited to, acetate, phosphate, succinate, histidine, tris(tris(hydroxymethyl)aminomethane), diethanolamine, citrate, other organic acids and mixtures thereof. The formulation can further comprise any counter-ion deemed suitable, such as sodium or calcium. In a preferred embodiment, the buffer is an acetate buffer (such as sodium acetate buffer). In another preferred embodiment, the buffer is an phosphate buffer (such as sodium phosphate buffer).

The buffer is present in an amount sufficient to maintain suitable pH. In some embodiments, the buffer is present in the formulations from about 0.1 mM to about 100 mM, from about 0.1 mM to about 50 mM, from about 0.1 mM to about 30 mM, about 0.1 mM to about 25 mM, from about 0.1 mM to about 20 mM, or from about 5 mM to about 15 mM. In certain embodiments, the buffer is at about 5 mM, 6 mM, 7 mM, 8 mM, 9 mM, 10 mM, 11 mM, 12 mM, 13 mM, 14 mM, or 15 mM. In some embodiments, the buffer is a sodium acetate buffer or a sodium phosphate buffer at about 10 mM.

The formulations can comprise a tonicity modifier that contributes to maintain the isotonicity of the formulation. In some embodiments, the formulation is isotonic, i.e., the formulation possesses the same or about the same osmotic pressure as blood plasma. Isotonic formulations will generally have an osmotic pressure from about 250 to 350 mOsm, preferably from about 250 to about 330 mOsm. In some embodiments, the formulation is hypertonic. In some embodiments, the formulation is hypotonic.

The tonicity modifier can be any tonicity modifier apparent to one of skill, such as a salt, a sugar, a sugar alcohol, a polyol or an amino acid. Exemplary tonicity modifiers include but are not limited to a salt such as sodium chloride, calcium chloride or magnesium chloride, a sugar or polyol such as lactose, sorbitol, sucrose, mannitol, trehalose, raffinose, polyethylene glycol, hydroxyethyl starch, glycine and combinations thereof. In some preferred embodiments, the tonicity modifier is sodium chloride. In other preferred embodiments, the tonicity modifier is sorbitol. In certain embodiments, combined tonicity modifiers yield a total osmolarity that is isotonic as described above.

When the formulation is a lyophilized formulation, salts or non-reducing sugars are preferred as tonicity modifiers. A “non-reducing sugar” is one which does not contain a hemiacetal group that can reduce metal ions or react covalently with lysine and other amino groups in proteins. Non-reducing sugars include sucrose, trehalose, sorbose, melezitose and raffinose. Non-reducing sugars can prevent or reduce chemical and/or physical instability of the peptides upon lyophilization and subsequent storage.

The tonicity modifier is present in the formulation in an amount to maintain desired tonicity of the formulation. In some embodiments, the tonicity modifier is present at about 0.1% to about 50% (w/v), about 0.5% to about 20% (w/v), about 1% to about 10% (w/v), or about 4% to about 6% (w/v). In some embodiments, the tonicity modifier is present at about 5% (w/v). In some embodiments, the tonicity modifier is present at a concentration of at least 1 mM. In some embodiments, the tonicity modifier is present at about 1 mM to about 200 mM, from about 10 mM to about 150 mM or from about 50 mM to about 100 mM. In some preferred embodiments, the formulation comprises about 135 mM sodium chloride. In other preferred embodiments, the formulation comprises about 150 mM sodium chloride. In other preferred embodiments, the formulation comprises about 5% sorbitol (w/v).

The formulations can also comprise a stabilizer to stabilize the conjugate during fluctuations in storage temperature and to minimize degradation products, peptide degradants and aggregation. Useful stabilizers in the formulations of the invention include, but are not limited to, sodium octanoate, Na—N-acetyltryptophan, H-glutamic acid, arginine, nitrogen and combinations thereof. In preferred embodiments, the stabilizer is sodium octanoate.

In certain embodiments, the stabilizer is present in the formulation at about 0.1 mM to 30 mM, about 0.5 mM and 20 mM, about 1 mM to about 15 mM, or about 5 mM to about 10 mM. In certain embodiments, the stabilizer is present in the formulation at about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM or 20 mM. In preferred embodiments, the stabilizer is sodium octanoate at about 5 mM.

The formulations can also comprise a surfactant. Surfactants are compounds that reduce interfacial tension between a liquid and a solid when dissolved in solution, and can be added to the formulation to reduce aggregation of the reconstituted protein and/or reduce the formation of particulates in the reconstituted formulation. Exemplary surfactants include polysorbates (e.g. polysorbates 20 or 80); poloxamers (e.g. poloxamer 188 (pluronic F68)); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethyl glycol, polypropyl glycol, and copolymers of ethylene and propylene glycol, etc.

The amount of surfactant is such that it reduces aggregation of the formulated peptides or peptide conjugates and/or minimizes the formation of particulates in the formulation and/or reduces adsorption. For example, the surfactant can be present in the formulation in an amount of about 0.001-1% (w/v), and preferably, about 0.01-0.5% (w/v). In some embodiments, the formulation comprises a surfactant which is a poloxamer. In some embodiments, the formulation comprises pluronic F68. In particular embodiments, the formulation comprises between about 0.01% (w/v) and about 1% (w/v) pluronic F68, more preferably about 0.1% (w/v) pluronic F68.

In certain embodiments, the formulations comprise the above-identified agents (i.e. insulinotropic peptide conjugates, buffer, tonicity modifier and surfactant) and are free of one or more preservatives, such as benzyl alcohol, phenol, m-cresol, chlorobutanol and benzethonium chloride. In other embodiments, a preservative can be included in the formulations, particularly where the formulations are multi-use formulations. Exemplary preservatives include but are not limited to m-cresol, benzyl alcohol, methanol, ethanol, iso-propanol, butyl paraben, ethyl paraben, methyl paraben, phenol, glycerol, xylitol, resorcinol, cathechol, 2,6-dimethylcyclohexanol, 2-methyl-2,4-pentadiol, dextran, polyvinylpyrrolidone, 2-chlorophenol, benzethonium chloride, merthiolate (thimerosal), benzoic acid (propyl paraben) MW 180.2, benzoic acid MW 122.12, benzalkonium chloride, chlorobutanol, sodium benzoate, sodium propionate, and cetylpyridinium chloride. Any of these preservatives can be used as a sole preservative or in combination with each other in the presently disclosed formulations.

In preferred embodiments, preservatives that are compatible with the buffer and other components of the formulations (i.e., the solution is clear) are used. When the buffer is sodium acetate or sodium phosphate, compatible preservatives include methanol, ethanol, iso-propanol, glycerol, resorcinol, 2-methyl-2,4-pentadiol, merthiolate (thimerosal), benzalkonium chloride, sodium benzoate, cetylpyridinium chloride.

The concentration of the preservative used in the formulations can be determined according to the judgment of those of skill in the art. In some embodiments, about 0.005 to 10% (w/v), about 0.1 to 1.0% (w/v), or about 0.3 to 0.7% (w/v) of the preservative is present in the formulations. In some embodiments, about 0.005, 0.1, 0.3, 0.5, 0.7, or 1.0% (w/v) of the preservative is present in the formulations.

A bulking agent can be included in a lyophilized formulation to facilitate the production of an essentially uniform lyophilized cake which maintains an open pore structure. Exemplary bulking agents include mannitol, glycine, polyethylene glycol and xorbitol. Bulking agents can also serve as a tonicity modifier as well.

One or more other pharmaceutically acceptable carriers, excipients or stabilizers, for example, such as described in Remington's Pharmaceutical Sciences 19th edition, Genarro, A. Ed. (1995) can be included in the formulations provided that they do not significantly adversely affect the desired characteristics of the formulation. Additional constituent elements of the formulations of the present invention can include water, e.g., water for injection, vegetable oil, a thickening agent such as methylcellulose antiadsorbant, a wetting agent, antioxidants including ascorbic acid and methionine, chelating agents such as EDTA, metal complexes (e.g. Zn-protein complexes), biodegradable polymers such as polyesters, and/or salt-forming counterions such as sodium etc. Acceptable carriers, excipients or stabilizers are present in an amount such that they are nontoxic to subjects at the dosages and concentrations employed.

The optimal formulation according to the present invention can vary depending on factors such as the amount of time the formulation will be stored, conditions under which the formulation will be stored and used, the particular subject population to which the formulation may be administered, etc.

In certain embodiments, the formulations as described herein can be contained in a vial, bottle, tube, syringe or other container for single or multiple administrations. Such containers can be made of glass or a polymer material such as polypropylene, polyethylene, polyvinylchloride, or polyolefin, for example. In some embodiments, the containers can include a seal, or other closure system, such as a rubber stopper that can be penetrated by a needle in order to withdraw a single dose and then re-seal upon removal of the needle. All such containers for injectable liquids, lyophilized formulations, reconstituted lyophilized formulations or reconstitutable powders for injection known in the art are contemplated for use in the presently disclosed formulations and methods. In a particular embodiment, the container is a pen-type delivery apparatus comprising a single dose or multiple doses. Such a pen-type delivery apparatus can be permanent, e.g., a permanent pen that houses a disposable cartridge containing a single dose or multiple doses, or the entire apparatus can be disposable, e.g., a disposable pen that contains a single dose or multiple doses. In certain embodiments where the pen-type delivery apparatus comprises multiple doses, the dose can be pre-set, i.e., fixed. In other embodiments, the dose can be a flexible dose, i.e., dialed-in by the user. In some embodiments, the pen-type delivery apparatus comprises a luer-lock, luer-cone, or other needle fitting connector that facilitates attachment of a disposable needle. In other embodiments, the pen-type delivery apparatus comprises a staked, i.e., permanent needle. In another particular embodiment, the container is a syringe. In some embodiments, the syringe comprises a luer-lock, luer-cone, or other needle fitting connector that facilitates attachment of a disposable needle. In other embodiments, the syringe comprises a staked, i.e., permanent, needle. In some embodiments, the syringe is prefilled with a single dose or multiple doses.

The formulations provided herein can be formulated in a variety of concentrations in various vial sizes for various administration dosages. For example, the dosages can be formulated in a 0.25, 0.5, 1 or 2 ml vial, or any other size vial or other container known by one of skill in the art.

The formulations to be used for in vivo administration must be sterile. This is readily accomplished by filtration through sterile filtration membranes, prior to, or following, preparation of the formulation. Alternatively, sterility of the entire formulation can be accomplished by autoclaving the ingredients, except for protein, at about 120° C. for about 30 minutes, for example.

In certain embodiments, the present invention provides a pharmaceutical formulation comprising a conjugate of albumin to exendin-4, or a derivative thereof, at a concentration from about 1 mg/ml to about 100 mg/ml, a buffer, a tonicity modifier, a stabilizer, a surfactant and optionally a preservative, wherein said formulation has a pH from about 4 to about 8.

In certain embodiments, the pharmaceutical formulation comprises, or alternatively consists of, a conjugate of albumin and an insulinotropic peptide, said insulinotropic peptide comprising a sequence which has not more than 3 amino acid substitutions, deletions, or insertions relative to the native exendin-4 sequence, said conjugate being at a concentration of about 1 mg/ml to about 100 mg/ml; a buffer; a tonicity modifier, wherein the tonicity modifier is at a concentration of at least 1 mM; a stabilizer; and a surfactant, wherein said formulation has a pH from about 4 to about 8.

In certain embodiments, the exendin-4 albumin conjugate comprises recombinant human serum albumin cysteine 34 thiol covalently linked to a [2-[2-[2 maleimidopropionamido(ethoxy)ethoxy]acetic acid linker on the epsilon amino of the carboxy terminal lysine of exendin-4(1-39)Lys⁴⁰-NH₂. Such a conjugate can be formed by covalently bonding the linker to the cysteine 34 thiol of the albumin. In some embodiments, the exendin-4 albumin conjugate is at a concentration of about 10 mg/ml to 20 mg/ml. In some embodiments, the buffer is a sodium acetate, or a sodium phosphate buffer or combinations thereof with a pH of about 5.0 to 6.0. In some embodiments, the tonicity modifier is sodium chloride or sorbitol. In some embodiments, the stabilizer is sodium octanoate. In some embodiments, the surfactant is pluronic F68.

In certain embodiments, the pharmaceutical formulation comprises, or alternatively consists of, about 1 mg/ml to about 15 mg/ml insulinotropic peptide conjugate in 5-30 mM sodium phosphate buffer at pH 6.5-7.5 containing 100-200 mM sodium chloride, 1-10 mM sodium octanoate, and 1-30 mg/L polysorbate 80. In a particular embodiment, the formulation comprises, or alternatively consists of, 10 mg/ml insulinotropic peptide conjugate in 5-30 mM sodium phosphate buffer at pH 6.5-7.5 containing 100-200 mM sodium chloride, 1-10 mM sodium octanoate, and 1-30 mg/L polysorbate 80. In a particular embodiment, the formulation comprises, or alternatively consists of, 10 mg/ml insulinotropic peptide conjugate in 10 mM sodium phosphate buffer containing 100-200 mM sodium chloride, 1-10 mM sodium octanoate, and 1-30 mg/L polysorbate 80 wherein said formulation has a pH of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. In a particular embodiment, the formulation comprises, or alternatively consists of, 10 mg/ml insulinotropic peptide conjugate in 10 mM sodium phosphate buffer at pH 7.0 containing 100-200 mM sodium chloride, 1-10 mM sodium octanoate, and 1-30 mg/L polysorbate 80. In a particular embodiment, the formulation comprises, or alternatively consists of, 10 mg/ml insulinotropic peptide conjugate in 10 mM sodium phosphate buffer at pH 7.0 containing 135 mM sodium chloride, 1.6 mM sodium octanoate, and 15 mg/L polysorbate 80.

In preferable embodiments, the pharmaceutical formulation comprises, or alternatively consists of, about 1 mg/ml to about 15 mg/ml exendin-4(1-39) Lys⁴⁰ (ε-AEEA-MPA)-NH₂ albumin conjugate in 5-30 mM sodium phosphate buffer at pH 6.5-7.5 containing 100-200 mM sodium chloride, 1-10 mM sodium octanoate, and 1-30 mg/L polysorbate 80. In a particular embodiment, the formulation comprises, or alternatively consists of, 10 mg/ml exendin-4(1-39) Lys⁴⁰ (ε-AEEA-MPA)-NH₂ albumin conjugate in 5-30 mM sodium phosphate buffer at pH 6.5-7.5 containing 100-200 mM sodium chloride, 1-10 mM sodium octanoate, and 1-30 mg/L polysorbate 80. In a particular embodiment, the formulation comprises, or alternatively consists of, 10 mg/ml exendin-4(1-39) Lys⁴⁰ (ε-AEEA-MPA)-NH₂ albumin conjugate in 10 mM sodium phosphate buffer containing 100-200 mM sodium chloride, 1-10 mM sodium octanoate, and 1-30 mg/L polysorbate 80 wherein said formulation has a pH of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. In a particular embodiment, the formulation comprises, or alternatively consists of, 10 mg/ml exendin-4(1-39) Lys⁴⁰ (ε-AEEA-MPA)-NH₂ albumin conjugate in 10 mM sodium phosphate buffer at pH 7.0 containing 100-200 mM sodium chloride, 1-10 mM sodium octanoate, and 1-30 mg/L polysorbate 80. In a particular embodiment, the formulation comprises, or alternatively consists of, 10 mg/ml exendin-4(1-39) Lys⁴⁰ (ε-AEEA-MPA)-NH₂ albumin conjugate in 10 mM sodium phosphate buffer at pH 7.0 containing 135 mM sodium chloride, 1.6 mM sodium octanoate, and 15 mg/L polysorbate 80.

In a particular embodiment, the formulation consists of about 1 mg/ml to about 15 mg/ml of an insulinotropic peptide conjugate in 10 mM sodium phosphate buffer at pH 7.0 containing 135 mM sodium chloride, 1.6 mM sodium octanoate, and 15 mg/L polysorbate 80. In a particular embodiment, the formulation consists of about 1 mg/ml to about 15 mg/ml of a conjugate of albumin to exendin-4, or a derivative thereof, in 10 mM sodium phosphate buffer at pH 7.0 containing 135 mM sodium chloride, 1.6 mM sodium octanoate, and 15 mg/L polysorbate 80. In a particular embodiment, the formulation consists of about 1 mg/ml to about 15 mg/ml exendin-4(1-39) Lys⁴⁰ (ε-AEEA-MPA)-NH₂ albumin conjugate in 10 mM sodium phosphate buffer at pH 7.0 containing 135 mM sodium chloride, 1.6 mM sodium octanoate, and 15 mg/L polysorbate 80. In a particular embodiment, the formulation consists of 10 mg/ml exendin-4(1-39) Lys⁴⁰ (ε-AEEA-MPA)-NH₂ albumin conjugate in 10 mM sodium phosphate buffer at pH 7.0 containing 135 mM sodium chloride, 1.6 mM sodium octanoate, and 15 mg/L polysorbate 80.

In certain embodiments, the pharmaceutical formulation comprises, or alternatively consists of, about 1 mg/ml to about 15 mg/ml insulinotropic peptide conjugate in 5-30 mM sodium acetate buffer at pH 4.5-5.5, containing 1-15 mM sodium octanoate, 0.05 to 0.2% (w/v) pluronic F68, and either 100-200 mM sodium chloride or 2-8% (w/v) sorbitol. In a particular embodiment, the formulation comprises, or alternatively consists of, 10 mg/ml insulinotropic peptide conjugate in 5-30 mM sodium acetate buffer at pH 4.5-5.5, containing I-15 mM sodium octanoate, 0.05 to 0.2% (w/v) pluronic F68, and either 100-200 mM sodium chloride or 2-8% (w/v) sorbitol. In a particular embodiment, the formulation comprises, or alternatively consists of, 10 mg/ml insulinotropic peptide conjugate in 10 mM sodium acetate buffer containing 1-15 mM sodium octanoate, 0.05 to 0.2% (w/v) pluronic F68, and either 100-200 mM sodium chloride or 2-8% (w/v) sorbitol wherein said formulation has a pH of about 4.5, 4.6, 4.7, 4.8, 4.9, 5.0, 5.1, 5.2, 5.3, 5.4, or 5.5. In a particular embodiment, the formulation comprises or alternatively consists of, 10 mg/ml insulinotropic peptide conjugate in 10 mM sodium acetate buffer at pH 5.0 containing 1-15 mM sodium octanoate, 0.05 to 0.2% (w/v) pluronic F68, and either 100-200 mM sodium chloride or 2-8% (w/v) sorbitol. In a particular embodiment, the formulation comprises, or alternatively consists of, 10 mg/ml insulinotropic peptide conjugate in 10 mM sodium acetate buffer at pH 5.0 containing 150 mM sodium chloride, 5 mM sodium octanoate and 0.1% (w/v) pluronic F68 (i.e., poloxamer 188).

In preferable embodiments, the pharmaceutical formulation comprises, or alternatively consists of, about 1 mg/ml to about 15 mg/ml exendin-4(1-39) Lys⁴⁰ (ε-AEEA-MPA)-NH₂ albumin conjugate in 5-30 mM sodium acetate buffer at pH 4.5-5.5, containing 1-15 mM sodium octanoate, 0.05 to 0.2% (w/v) pluronic F68, and either 100-200 mM sodium chloride or 2-8% (w/v) sorbitol. In a particular embodiment, the formulation comprises, or alternatively consists of, 10 mg/ml exendin-4(1-39) Lys⁴⁰ (ε-AEEA-MPA)-NH₂ albumin conjugate in 5-30 mM sodium acetate buffer at pH 4.5-5.5, containing 1-15 mM sodium octanoate, 0.05 to 0.2% (w/v) pluronic F68, and either 100-200 mM sodium chloride or 2-8% (w/v) sorbitol. In a particular embodiment, the formulation comprises, or alternatively consists of, 10 mg/ml exendin-4(1-39) Lys⁴⁰ (ε-AEEA-MPA)-NH₂ albumin conjugate in 10 mM sodium acetate buffer containing 1-15 mM sodium octanoate, 0.05 to 0.2% (w/v) pluronic F68, and either 100-200 mM sodium chloride or 2-8% (w/v) sorbitol wherein said formulation has a pH of about 4.5, 4.6, 4.7, 4.8, 4.9, 5.0, 5.1, 5.2, 5.3, 5.4, or 5.5. In a particular embodiment, the formulation comprises, or alternatively consists of, 10 mg/ml exendin-4(1-39) Lys⁴⁰ (ε-AEEA-MPA)-NH₂ albumin conjugate in 10 mM sodium acetate buffer at pH 5.0 containing 1-15 mM sodium octanoate, 0.05 to 0.2% (w/v) pluronic F68, and either 100-200 mM sodium chloride or 2-8% (w/v) sorbitol. In a particular embodiment, the formulation comprises, or alternatively consists of, 10 mg/ml exendin-4(1-39) Lys⁴⁰ (ε-AEEA-MPA)-NH₂ albumin conjugate in 10 mM sodium acetate buffer at pH 5.0 containing 150 mM sodium chloride, 5 mM sodium octanoate and 0.1% (w/v) pluronic F68 (i.e., poloxamer 188).

In a particular embodiment, the formulation consists of about 1 mg/ml to about 15 mg/ml of an insulinotropic peptide conjugate in 10 mM sodium acetate buffer at pH 5.0 containing 150 mM sodium chloride, 5 mM sodium octanoate and 0.1% (w/v) pluronic F68 (i.e., poloxamer 188). In a particular embodiment, the formulation consists of about 1 mg/ml to about 15 mg/ml of a conjugate of albumin to exendin-4, or a derivative thereof, in 10 mM sodium acetate buffer at pH 5.0 containing 150 mM sodium chloride, 5 mM sodium octanoate and 0.1% (w/v) pluronic F68 (i.e., poloxamer 188). In a particular embodiment, the formulation consists of about 1 mg/ml to about 15 mg/ml exendin-4(1-39) Lys⁴⁰ (ε-AEEA-MPA)-NH₂ albumin conjugate in 10 mM sodium acetate buffer at pH 5.0 containing 150 mM sodium chloride, 5 mM sodium octanoate and 0.1% (w/v) pluronic F68 (i.e., poloxamer 188). In a particular embodiment, the formulation consists of 10 mg/ml exendin-4(1-39) Lys⁴⁰ (ε-AEEA-MPA)-NH₂ albumin conjugate in 10 mM sodium acetate buffer at pH 5.0 containing 150 mM sodium chloride, 5 mM sodium octanoate and 0.1% (w/v) pluronic F68 (i.e., poloxamer 188).

The pharmaceutical formulations provided herein can be in any form deemed useful to those of skill in the art. For instance, they can be in the form of liquid or lyophilized formulations, unit dosage forms or multi-use dosage forms and combinations thereof. Thus, the formulations include liquid unit dosage forms, liquid multi-use forms, lyophilized unit dosage forms and lyophilized multi-use dosage forms.

In some embodiments, the formulation is a liquid formulation. In other embodiments, the formulation is a lyophilized formulation. Lyophilization is a commonly employed technique for preserving proteins which serves to remove water from the peptide preparation of interest. An excipient can be included in pre-lyophilized formulations to enhance stability during the freeze-drying process and/or to improve stability of the lyophilized product upon storage. See Pikal, M. 1990, Biopharm. 3(9):26-30 and Arakawa et al 1991, Pharm. Res. 8(3):285-291.

Lyophilized formulations can be reconstituted according to the judgment of those of skill in the art. In preferred embodiments, a lyophilized formulation is provided which, when reconstituted, e.g., with water for injection, results in one of the liquid formulations described herein. The present invention also provides a method of reconstituting a lyophilized formulation of an insulinotropic peptide conjugate comprising providing the lyophilized formulation, and reconstituting the lyophilized formulation to form an insulinotropic peptide conjugate formulation described herein.

At the desired stage, typically when it is time to administer the peptide to the subject, the lyophilized formulation can be reconstituted with a diluent such that the protein concentration in the reconstituted formulation is at least 1, 2, 3, 4, 5, 10, 20, 30, 40, 50 mg/ml. In some embodiments, the protein concentration in the reconstituted formulation is from about 1 mg/ml to about 100 mg/ml, from about 1 mg/ml to about 50 mg/ml, or from about 1 mg/ml to about 15 mg/ml. In particular embodiments, the lyophilized formulation can be reconstituted with a diluent such that the protein concentration in the reconstituted formulation is about 45-55 mg/ml. In preferred embodiments, the lyophilized formulation can be reconstituted with a diluent such that the protein concentration in the reconstituted formulation is about 50 mg/ml. The diluent can be any diluent deemed suitable by one of skill, e.g., water for injection, and the like.

The pharmaceutical formulations provided herein include both unit dosage forms and multi-use dosage forms. In some embodiments, the formulations are in unit dosage forms. “Unit dosage form” refers to a packaged form of the pharmaceutical formulation in an amount that is intended for a single administration to a subject. In some embodiments, the formulations are in unit dosage forms. In certain embodiments, the unit dosage comprises about 0.01-100 mg, 0.1-50 mg, 1-10 mg, or 1-5 mg insulinotropic peptide conjugate. In particular embodiments, the unit dosages comprise about 1, 2, 3, 4, 5, 7.5, 10, 20, 30, 40, 50, 75, 100 mg insulinotropic peptide conjugate. Such unit dosages can be prepared according to techniques known to those of skill in the art.

In some embodiments, the formulations are in multi-use dosage forms. Multi-use formulations can facilitate ease of use for subjects, reduce waste by allowing complete use of vial contents and result in significant cost savings for manufacture. Multi-use pharmaceutical formulations can be contained in multi-dose containers, e.g., vials, ampoules, etc., that allow for the extraction of partial amounts of the formulations at various times. One or more preservatives compatible with the buffer in the formulations can be present in multi-use formulations as described in detail above.

Preferably, the formulations of the present invention are stable. In some embodiments, the formulations are stable for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more than 36 months at a temperature of about 4° C. In other embodiments, the formulations are stable for at least about 1, 2 or 3 weeks, or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or more than 36 months at a temperature of about 25° C. In other embodiments, the formulations are stable for at least about 1, 2 or 3 weeks, or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or more than 36 months at a temperature of about 40° C.

5.2.1 Preparation of the Pharmaceutical Formulations

Formulations provided herein can be prepared by any technique apparent to one of skill in the art. In certain embodiments, a formulation can be prepared by contacting an insulinotropic peptide conjugate with other components of the formulation under conditions suitable for preparation of the formulation. For instance, the insulinotropic peptide conjugate can be mixed with the other components, dialyzed with the other components, diafiltered with the other components, or contacted with the other components by any technique apparent to one of skill in the art. The insulinotropic peptide conjugate can be prepared by any technique apparent to one of skill in the art. Exemplary techniques are described herein. The insulinotropic peptide conjugate can be purified according to any method deemed suitable by one of skill in the art. Exemplary methods are described herein.

The insulinotropic peptide conjugates of the formulations of the present invention can be purified according to any purification method known in the art prior to formulation in a desired formulation composition. In some embodiments, the conjugate is purified by hydrophobic interaction chromatography (HIC). The HIC can be any HIC technique known to those of skill. In certain embodiments, the conjugate can be purified by two HIC purifications, e.g., two HIC purifications in sequence.

In one embodiment, a first purification step comprises contacting an insulinotropic peptide conjugate with phenyl sepharose, i.e., a bead-formed agarose-based gel filtration matrix covalently coupled to a phenyl group. In certain embodiments, this step separates non-conjugated insulinotropic peptide from albumin species, whether free or conjugated. In certain embodiments, the phenyl sepharose is equilibrated with a phosphate buffer of pH 6.0 comprising 5 mM sodium octanoate and 5 mM ammonium sulfate. Under these conditions, non-conjugated compound is capable of binding to the phenyl sepharose while the conjugate is capable of flowing through the phenyl sepharose. The conjugate can then be collected as the flow through fraction for further purification.

In certain embodiments, purification of the conjugate further comprises a second HIC step wherein the phenyl sepharose flow-through is contacted with butyl sepharose, i.e., a bead-formed agarose-based gel filtration matrix covalently coupled to a butyl group, to further purify the conjugate from non-conjugated albumin, dimeric non-conjugated albumin, and residual non-conjugated compound. In certain embodiments, the butyl sepharose is equilibrated in a buffer at or near pH 6.0 comprising 5 mM sodium octanoate and 750 mM ammonium sulfate. The butyl sepharose is then contacted with the phenyl sepharose flow-through of the first purification step described above. In certain embodiments, elution of the conjugate can be achieved using either a linear or stepwise decreasing salt gradient, or a combination thereof, wherein non-conjugated albumin can be eluted with about 750 mM ammonium sulfate, dimeric non-conjugated albumin can be eluted with about 550 mM ammonium sulfate, compound-albumin conjugates (the desired species) can be eluted with about 100 mM ammonium sulfate, and unconjugated compound and other species can be eluted with water or an equivalent thereof. These species can include, for example, dimeric, trimeric, or polymeric albumin conjugates, or albumin conjugate products comprising a stoichiometry of compound to albumin greater than 1:1.

In certain embodiments, purification of the conjugate further comprises washing and concentrating the conjugate by ultrafiltration following HIC. In some embodiments, sterile water, saline, or buffer can be used to remove ammonium sulfate and buffer components from the purified conjugate.

In other embodiments, insulinotropic peptide conjugates can be purified according to the purification methods described in U.S. patent application Ser. No. 11/645,297 (Publication No. 2007/0269863), filed Dec. 22, 2006, entitled “Process for the Production of Preformed Conjugates of Albumin and a Therapeutic Agent,” which is incorporated by reference herein in its entirety.

In certain embodiments, following purification of the insulinotropic peptide conjugate, the conjugate can be reformulated in a desired formulation composition, e.g., a formulation of the present invention by any technique apparent to one of skill. See Remington's Pharmaceutical Sciences 16th edition, Osol, A. Ed. (1980). For example, liquid formulations can be prepared by mixing the components in a container and adding water or buffers to the desired volume and concentration. Other exemplary techniques include dialysis, ultrafiltration, diafiltration, size exclusion chromatography, etc. Generally, the conjugate can be contacted with formulation components under conditions that yield a formulation provided herein.

In certain embodiments, reformulation of the purified insulinotropic peptide conjugate comprises pooling into a suitable container fractions which contain the insulinotropic peptide conjugate eluted from the second HIC purification step described above, i.e., following butyl sepharose chromatography. The pooled material can then be concentrated using any concentration method known in the art. In certain embodiments, the pooled material can be concentrated using an ultrafiltration membrane and pumping system until a protein concentration of about 10, 20, 30, 40, 50, 60, 70, 80, 90, 100 or more than 100 mg/ml is achieved. In a particular embodiment, the pooled material is concentrated to a protein concentration of about 70 mg/ml. The concentrated product can then be diafiltered against at least 1, 2, 3, 4, 5, 6, 7, 8, 9, 10 or more than 10 volumes of water, wherein the volume of the solution containing insulinotropic peptide conjugate is kept constant. In particular embodiments, the concentrated product is diafiltered against at least 10 volumes of water. In some embodiments, the diafiltered solution comprising the insulinotropic peptide conjugate can then be contacted, i.e., mixed with a desired formulation composition to achieve a formulation composition comprising the insulinotropic peptide conjugate. In particular embodiments, a 5× concentration of the desired formulation composition can be prepared, and 4 parts solution containing the insulinotropic peptide conjugate can be mixed with 1 part 5× formulation solution to achieve an insulinotropic peptide conjugate formulation described herein. In certain embodiments, the protein concentration of the resulting solution can be measured, and the protein concentration can be adjusted as required with formulation buffer to achieve a desired concentration of the insulinotropic peptide conjugate in 1× formulation buffer. In some embodiments, the final concentration of the insulinotropic peptide conjugate in 1× formulation buffer is about 1, 10, 20, 30, 40, 50, 60, 70, 80, 90, 100 or more than 100 mg/ml. In particular embodiments, the final concentration of the insulinotropic peptide conjugate in 1× formulation buffer is about 10 mg/ml. In another particular embodiment, the final concentration of the insulinotropic peptide conjugate in 1× formulation buffer is about 50 mg/ml. The product can be further filtered according to any method known in the art before preparing for storage.

In an alternative embodiment, reformulation of the purified insulinotropic peptide conjugate can comprise the following steps. By way of example and not limitation the following is presented. Following pooling of the fractions obtained from the second HIC purification step, i.e., after butyl sepharose chromatography, and concentration of the insulinotropic peptide conjugate to about 70 mg/ml, as described above, the concentrated product can then be diafiltered against at least 10 volumes of a diafiltration buffer comprising a desired formulation composition of the present invention, wherein the formulation composition does not include the surfactant poloxamer 188 (pluronic F68). The concentrated product can be diafiltered against at least 10 volumes of diafiltration buffer, wherein the volume of the solution containing insulinotropic peptide conjugate is kept constant. Where appropriate, a “5× poloxamer 188 solution,” comprising a 5× concentration of the surfactant poloxamer 188, e.g., 0.5% (w/v) poloxamer 188, can then be prepared in the diafiltration buffer described above, and 4 parts solution containing the insulinotropic peptide conjugate can be mixed with 1 part 5× poloxamer 188 solution. The protein concentration of the resulting solution can be measured, and the protein concentration can be adjusted as required with formulation buffer to achieve a concentration of about 50 mg/ml insulinotropic peptide conjugate in 1× formulation buffer. The product can be further filtered according to any method known in the art before preparing for storage.

In other embodiments, lyophilized formulations can be prepared by contacting the peptide or peptide conjugate with other components and lyophilizing the resulting mixture. Many freeze-dryers are available for this purpose such as Hull50™ (Hull, USA) or GT20™ (Leybold-Heraeus, Germany) freeze-dryers. Freeze-drying can be accomplished by freezing the formulation and subsequently subliming ice from the frozen content at a temperature suitable for primary drying. Under this condition, the product temperature is below the eutectic point or the collapse temperature of the formulation. Typically, the shelf temperature for the primary drying will range from about −30 to −5° C. (provided the product remains frozen during primary drying) at a suitable pressure, ranging typically from about 50 to 250 mTorr. The formulation, size and type of the container holding the sample (e.g., glass vial) and the volume of liquid will mainly dictate the time required for drying, which can range from a few hours to several days (e.g. 40-60 hrs). A secondary drying stage can be carried out at about 0 to 40° C. depending primarily on the type and size of container and the type of protein employed. However, in certain embodiments, a secondary drying step might not be necessary. For example, the shelf temperature throughout the entire water removal phase of lyophilization can be from about −30 to −5° C. The time and pressure required for secondary drying will be that which produces a suitable lyophilized cake, dependent, e.g., on the temperature and other parameters. The secondary drying time is dictated by the desired residual moisture level in the product and typically takes at least about 5 hours (e.g. 10-15 hours). The pressure can be the same as that employed during the primary drying step. Freeze-drying conditions can be varied depending on the formulation and vial size.

5.2.1.1 Evaluation of Prepared Formulations

In one aspect, the invention provides methods of evaluating a sample of an insulinotropic peptide conjugate, e.g., exendin-4(1-39) Lys⁴⁰ (ε-AEEA-MPA)-NH₂ albumin conjugate prepared and/or formulated according to the methods provided herein to determine the levels of one or more species in the sample. In certain embodiments, the methods comprise: determining a value for the level of one or more species in a sample containing an insulinotropic peptide conjugate, e.g., exendin-4(1-39) Lys⁴⁰ (ε-AEEA-MPA)-NH₂ albumin conjugate; and comparing the value to a reference value, thereby evaluating the sample. The reference value can be any predetermined value or range of values, e.g., a value which has been set by a government agency, e.g., the FDA, or another party, e.g., the manufacturer of an approved preparation of the insulinotropic peptide conjugate or by a compendial authority, e.g., the USP.

The species can be any species that one of skill in the art might evaluate in the sample. Examples include, but are not limited to, the insulinotropic peptide conjugate, unconjugated albumin and unconjugated insulinotropic peptide, or any derivative of such species. In certain embodiments, the derivative of the unconjugated insulinotropic peptide can be an oxidized peptide, e.g. oxidized at a methionine residue, a deaminated peptide, e.g. deaminated at an asparagine or glutamine residue, or an oxidized and deaminated peptide. In certain embodiments, the species is a conjugate of multiple insulinotropic peptides with a macromolecule (for example; albumin), e.g. 2:1 peptide to macromolecule or 3:1 peptide to macromolecule or 4:1 peptide to macromolecule.

In a preferred embodiment, the species is exendin-4(1-39) Lys⁴⁰ (ε-AEEA-MPA)-NH₂ albumin conjugate.

In a preferred embodiment, the species evaluated is unconjugated albumin. In preferred embodiments, the value for the level of unconjugated albumin in the sample is <10.0 mg/ml.

In a preferred embodiment, the species evaluated is unconjugated exendin-4(1-39) Lys⁴⁰ (ε-AEEA-MPA)-NH₂. In preferred embodiments the value for the level of unconjugated exendin-4(1-39) Lys⁴⁰ (ε-AEEA-MPA)-NH₂ is <25.0 μg/ml.

In a particular embodiment, the species evaluated is exendin-4(1-39) Lys⁴⁰ (ε-AEEA-MPA)-NH₂ conjugated to albumin at a ratio of 2:1.

In a particular embodiment, the species evaluated is exendin-4(1-39) Lys⁴⁰ (ε-AEEA-MPA)-NH₂ conjugated to albumin at a ratio of 3:1.

Any method known in the art can be used to determine a value of an species in a sample comprising an insulinotropic peptide conjugate. In some embodiments, the level of an unconjugated species in a sample is determined by gel electrophoresis, liquid chromatography-mass spectrometry (LCMS), hydrophobic interaction chromatography, high performance liquid chromatography (HPLC), reverse phase chromatography, e.g. reverse phase HPLC, circular diochroism, melting temperature, osmolality, or ultraviolet absorbance, e.g. absorbance at 280 nm.

In certain embodiments, hydrophobic interaction chromatography is useful for detecting or quantifying conjugate, unconjugated albumin, unconjugated peptide and/or conjugates of multiple insulinotropic peptides with a macromolecule.

In certain embodiments, gel electrophoresis is useful for detecting or quantifying conjugate, unconjugated albumin, unconjugated peptide and/or conjugates of multiple insulinotropic peptides with a macromolecule. In certain embodiments, gel electrophoresis can be combined with immunological detection, e.g. western blot or enzyme-linked immunosorbent assay, to facilitate detection.

In certain embodiments, LCMS is useful for detecting conjugate, unconjugated albumin, unconjugated peptide and/or conjugates of multiple insulinotropic peptides with a macromolecule.

In certain embodiments, reverse phase HPLC is useful for detecting or quantifying unconjugated peptide and/or derivatives of unconjugated peptide.

### 5.3 Methods of Treatment

Also provided herein are methods of treating in a subject a disorder or condition treatable with an insulinotropic peptide. In certain embodiments, the disorder or condition treatable with an insulinotropic peptide is obesity. In certain embodiments, the disorder or condition treatable with an insulinotropic peptide is diabetes. While not wishing to be bound by theory, it is believed that the pharmaceutical formulations provided herein will normalize hyperglycemia through glucose-dependent, insulin-dependent and insulin-independent mechanisms. The pharmaceutical formulations are useful as primary agents for the treatment of type II diabetes mellitus and as adjunctive agents for the treatment of type I diabetes mellitus. In certain embodiments, the disorder or condition treatable with an insulinotropic peptide is type II diabetes. In some embodiments, the methods comprise the step of administering to the subject a therapeutically effective amount of an insulinotropic peptide conjugate, e.g. an insulinotropic peptide conjugate formulation described herein. In some embodiments, the insulinotropic peptide conjugate is a conjugate of albumin to exendin-4, or a derivative thereof. In preferred embodiments, the subject is a human.

The pharmaceutical formulations are especially suited for the treatment of subjects with diabetes, both type I and type II, in that the action of the peptide is dependent on the glucose concentration of the blood, and thus the risk of hypoglycemic side effects are greatly reduced over the risks in using current methods of treatment.

Thus, in certain aspects, provided herein are methods of treating type II diabetes mellitus in a subject, comprising administering to a subject having type II diabetes mellitus a formulation described herein. In some embodiments, the formulation comprises a conjugate of albumin and an insulinotropic peptide, said insulinotropic peptide comprising a sequence which has not more than 3 amino acid substitutions, deletions, or insertions relative to the native exendin-4 sequence, said conjugate being at a concentration of about 1 mg/ml to about 100 mg/ml; a buffer; a tonicity modifier; a stabilizer; and a surfactant, wherein said formulation has a pH from about 4 to about 8. In certain embodiments, the method comprises administering to a subject having type II diabetes mellitus a formulation comprising an insulinotropic conjugated exendin-4 derivative, the derivative comprising recombinant human serum albumin cysteine 34 thiol covalently linked to a [2-[2-[2 maleimidopropionamido(ethoxy)ethoxy]acetic acid linker covalently linked to the epsilon amino of the carboxy terminal lysine of exendin-4(1-39)Lys⁴⁰-NH₂.

The pharmaceutical formulations of the present invention can also be used for the treatment of subjects with obesity. The pharmaceutical formulations of the present invention can also be used for the treatment of subjects with any disorder or disease treatable with an insulinotropic peptide.

5.3.1 Subjects

In certain embodiments of the invention, the subject is an animal, for example, a mammal, e.g., a non-human primate. In certain embodiments, the subject is a human. The subject can be a male or female subject. In certain embodiments, the subject is a non-human animal, such as, for instance, a cow, sheep, goat, horse, cat or dog.

In certain embodiments, the subject is at risk for a disorder or a condition treatable with an insulinotropic peptide including, but not limited to, obesity and type II diabetes. In some embodiments the subject is at risk for obesity. In some embodiments the subject is at risk for type II diabetes.

In some embodiments, the subject is not healthy. In some embodiments the subject has or suffers from a condition treatable with an insulinotropic peptide including, but not limited to, obesity or type II diabetes.

In some embodiments, the subject is obese. In some embodiments, the subject is a human and has a Body Mass Index (BMI) of 30 kg/m² or greater. In some embodiments, the subject is a human and has a BMI between 30 kg/m² and 35 kg/m². In some embodiments, the subject is a human and has a BMI of 35 kg/m² or greater. In some embodiments, the subject is a human and has a BMI of 40 kg/m² or greater. In some embodiments, the subject weighs more than 120% of the normal weight for its age and height and/or ethnicity.

In some embodiments, the subject has type II diabetes. In some embodiments, the subject has abnormal glucose levels. In particular embodiments, the subject has a high glucose level. In some embodiments, the subject is a human and has an average whole blood glucose level of 8 mmol/L (138 mg/dl) or greater, and/or an average plasma blood glucose level of 9.0 mmol/L (154 mg/dl) or greater. In some embodiments, the subject is a human and has an average whole blood glucose level between 8 mmol/L (138 mg/dl) and 16 mmol/L (281 mg/dl), and/or an average plasma blood glucose level between 9.0 mmol/L (154 mg/dl) and 17 mmol/L (314 mg/dl). In some embodiments, the subject is a human and has an average whole blood glucose level greater than 16 mmol/L (281 mg/dl), and/or an average plasma blood glucose level greater than 17 mmol/L (314 mg/dl).

In some embodiments, the subject is a human and has a glycosylated hemoglobin (HbA1c) level of 6.5% or greater. In some embodiments, the subject is a human and has a HbA1c level between 6.5% and 11%. In some embodiments, the subject is human and has a HbA1c level of 11% or greater.

In certain embodiments, the subject has a disease, disorder or condition treatable with an insulinotropic peptide, e.g., an insulinotropic peptide conjugate. For instance, the subject has Metabolic Syndrome. According to the Third Report of the National Cholesterol Education Program's Adult Treatment Panel (ATPIII), a subject with Metabolic Syndrome has three or more of the following criteria: (1) waist circumference of greater than 102 cm for men and greater than 88 cm for women; (2) serum triglycerides of greater than 1.7 mmol/l; (3) blood pressure of greater than 130/85 mmHg; (4) HDL-cholesterol of less than 1.0 mmol/l in men and less than 1.3 mmol/l in women; and (5) serum glucose of greater than 6.1 mmol/l (greater than 5.6 mmol/1 may be applicable). According to the World Health Organization (WHO), a subject with Metabolic Syndrome has diabetes or impaired fasting glucose (IFG) or impaired glucose tolerance (IGT) or insulin resistance (assessed by clamp studies), plus at least two of the following criteria: (1) waist-to-hip ratio of greater than 0.90 in men or greater than 0.85 in women; (2) serum triglycerides of greater than 1.7 mmol/l or HDL-cholesterol of less than 0.9 mmol/l in men and less than 1.0 mmol/l in women; (3) blood pressure of greater than 140/90 mmHg; (4) urinary albumin excretion rate of greater than 20 micrograms/minute or albumin:creatinine ratio of greater than 30 mg/g. Thus, if a subject meets the criteria defined by either the ATPIII or WHO for Metabolic Syndrome, then the subject has Metabolic Syndrome.

In some embodiments, the subject has pre-diabetes (e.g., impaired glucose tolerance (IGT) or impaired fasting glucose (IFG)). In some embodiments, the subject has diabetes, e.g., type I diabetes, type II diabetes. In some embodiments, the subject has late autoimmune diabetes in adults (“LADA”) also known as late onset autoimmune diabetes of adulthood. In some embodiments, the subject has slow onset type I diabetes. In some embodiments, the subject has type 1.5 diabetes. In some embodiments, the subject has steroid induced diabetes. In some embodiments, the subject has Human Immunodeficiency Virus (HIV) Treatment-Induced Diabetes. In some embodiments, the subject has congenital or HIV-Associated Lipodystrophy (“Fat Redistribution Syndrome”) related diabetes. In some embodiments, the subject has a nervous system disorder. In some embodiments, the subject has insulin resistance. In some embodiments, the subject has hypoglycemia unawareness. In some embodiments, the subject has restrictive lung disease. In some embodiments, the subject has gastrointestinal disorders, e.g., irritable bowel syndrome (IBS), functional dyspepsia, or pain associated with gastrointestinal disorders, e.g., pain associated with IBS and functional dyspepsia. In some embodiments, the subject has inflammatory bowel disease (IBD), e.g., Crohn's disease and ulcerative colitis, or pain associated with IBD. In some embodiments, the subject has hyperglycemia, e.g., hyperglycemia associated with surgery (e.g., a major surgical procedure, e.g., coronary bypass surgery) e.g., hyperglycemia associated with surgery on subjects with diabetes, e.g., type II diabetes or metabolic syndrome. In some embodiments, the subject has coronary heart failure (CHF). In some embodiments, the subject has disorders associated with beta cell disfunction, disorders associated with the absence of beta cells, or disorders associated with insufficient numbers of beta cells.

In some embodiments, the subject is obese. In some embodiments, the subject is obese but neither diabetic nor pre-diabetic; obese and diabetic or pre-diabetic; obese but not affected by metabolic syndrome; obese and affected by the metabolic syndrome; overweight but neither diabetic nor pre-diabetic; overweight and diabetic or pre-diabetic; overweight but not affected by metabolic syndrome; overweight and affected by metabolic syndrome; affected by metabolic syndrome but neither diabetic nor pre-diabetic (depending on the definition of metabolic syndrome); affected by metabolic syndrome but neither obese nor overweight.

In some embodiments, the subject has one or more of the following characteristics: (1) diabetes or pre-diabetes; (2) overweight or obese; and (3) metabolic syndrome.

In some embodiments, the subject is naive to anti-diabetic agents. In some embodiments, the subject is naive to other anti-diabetic agents or naïve to oral anti-diabetic agents (OAD). In other embodiments, the subject has been previously treated with one or more other antidiabetic agents, e.g., an OAD. In other embodiments, the subject has been previously treated with metformin, a sulfonylurea, a thiazolidinedione or a combination thereof. In some embodiments, the subject is being treated with, i.e., on an active treatment regimen with an OAD. In one embodiment, the subject has been administered an OAD, e.g. metformin within 1 week, 2 days, or 1 day prior to the administration of the insulinotropic peptide conjugate. In a specific embodiment, the subject has been on a stable dose of ≧1000 mg metformin daily for at least 3 months. Exemplary OADs are provided below.

In a particular embodiment, the subject is currently being treated with, i.e., on an active treatment regimen with metformin. In one embodiment, the subject has been administered metformin within 1 week, 2 days, or 1 day prior to the administration of the insulinotropic peptide conjugate. In a particular embodiment, the subject has been on a stable dose of ≧1000 mg metformin daily for at least 3 months.

In certain embodiments, the formulations herein can be administered as monotherapy. In other words, the formulations herein can be provided as the sole administration of an active agent for treatment of one or more conditions provided herein.

5.3.2 Combination Therapies with Antidiabetic Agents

In the methods and formulations provided herein, an insulinotropic peptide conjugate can be used with or combined with one or more second therapeutic agents in the treatment or prevention of diabetes, obesity, or disorders treatable with an insulinotropic peptide, e.g., an insulinotropic peptide conjugate. In some embodiments, the combinations of these agents can produce a more effective treatment for such diseases or disorders than with either single treatment alone.

A formulation provided herein can be combined with a second therapeutic agent by any means deemed suitable by a practitioner of skill in the art. For instance, the formulation can be administered as described herein, and the second therapeutic agent can be administered according to any means and according to any schedule and dose suitable for that agent. Methods of administration, doses, and dose schedules are within the skill of those in the art and can be determined based on knowledge of the second active agent. In certain embodiments, doses and dose schedules can be adjusted for combination therapy by those of skill in the art. The formulation and the second agent need not be administered together. However, in certain embodiments, where suitable, the formulation and the second agent can be administered together where appropriate. In certain embodiments, the formulation can comprise the second agent in addition to the insulinotropic peptide where appropriate.

One or more second therapeutic ingredients or agents can be used together with an insulinotropic peptide conjugate in the methods provided herein. Second therapeutic agents include anti-diabetic agents, including oral-anti-diabetic agents (OADs) or anti-obesity agents.

5.3.2.1 OADs

Exemplary OADs which find use in the combination therapies provided herein include, but are not limited to, sulfonylureas, e.g. tolbutamide (Orinase), acetohexamide (Dymelor), tolazamide (Tolinase), chlorpropamide, (Diabinese), glipizide (Glucotrol), glyburide (Diabeta, Micronase, Glynase), glibenclamide, glimepiride (Amaryl) or gliclazide (Diamicron); biguanides, e.g. metformin, phenformin or buformin; glinide, e.g., Starlix (nateglinide), Prandin (repaglinide), Glufast (mitiglinide); meglitinides, e.g. repaglinide (Prandin) or nateglinide (Starlix); thiazolidinediones, e.g. rosiglitazone (Avandia), pioglitazone (Actos) or troglitazone (Rezulin); or Alpha-glucosidase inhibitors, e.g. miglitol (Glyset) or acarbose (Precose/Glucobay).

5.3.2.2 DPP IV Inhibitors

In some embodiments, the second therapeutic agent which finds use in the combination therapies provided herein is a dipeptidyl peptidase IV inhibitor (DPP IV inhibitor). The DPP-IV inhibitor can be any compound that exhibits inhibition of the enzymatic activity of DPP-IV. Examples of DPP-IV inhibitors are described, for example, in (i) D. J. Drucker, 2003, Exp. Opin. Invest. Drugs, 12:87-100; (ii) K. Augustyns, et al., 2003, Exp. Opin. Ther. Patents, 13:499-510; (iii) C. F. Deacon, et al., 2004, Exp. Opin. Investig. Drugs, 13:1091-1102; (iv) A. E. Weber, 2004, J. Med. Chem., 47:4135-4141; (v) J. J. Holst, 2004, Exp. Opin. Emerg. Drugs, 9: 155-166; (vi) Augustyns et al., 2005, Expert Opinion On Therapeutic Patents, 15(10):1387-1407; (vii) Sebokova et al., 2007, Current Topics in Medicinal Chemistry 7:547-555, the contents of each of which are incorporated by reference herein in their entireties.

Where the DPP IV inhibitor is orally available or orally administered, the DPP IV inhibitor is an OAD as described herein. In other words, OADs can include some or all DPP IV inhibitors described herein.

Specific examples of DPP-IV inhibitors include, but are not limited to, dipeptide derivatives or dipeptide mimetics such as alanine-pyrrolidide, isoleucine-thiazolidide, and the pseudosubstrate N-valyl prolyl, O-benzoyl hydroxylamine, as described e.g. in U.S. Pat. Nos. 7,253,172, 7,241,756, 7,238,724, 7,238,720, 7,236,683, 7,235,538, 7,230,074, 7,230,002, 7,229,969, 7,223,573, 7,217,711, 7,208,498, 7,205,409, 7,205,323, 7,196,201, 7,192,952, 7,189,728, 7,186,846, 7,186,731, 7,183,290, 7,183,280, 7,179,809, 7,169,926, 7,169,806, 7,166,579, 7,157,490, 7,144,886, 7,132,443, 7,125,873, 7,125,863, 7,122,555, 7,115,650, 7,109,192, 7,101,871, 7,098,239, 7,084,120, 7,078,397, 7,078,281, 7,074,794, 7,060,722, 7,053,055, 7,034,039, 7,026,316, 6,911,467, 6,890,898, 6,890,905, 6,869,947, 6,867,205, 6,861,440, 6,844,316, 6,849,622, 6,825,169, 6,812,350, 6,803,357, 6,800,650, 6,727,261, 6,716,843, 6,710,040, 6,706,742, 6,699,871, 6,645,995, 6,617,340, 6,699,871, 6,573,287, 6,432,969, 6,395,767, 6,380,398, 6,319,893, 6,303,661, 6,242,422, 6,201,132, 6,172,081, 6,166,063, 6,124,305, 6,110,949, 6,107,317, 6,100,234, 6,040,145, 6,011,155, 5,939,560, 5,462,928, the contents of each of which are incorporated by reference herein in their entireties.

Further examples of DPP-IV inhibitors can be found in U.S. Pat. App. Pub. Nos. 20070172525, 20070185061, 2007016750, 20070149451, 20070142383, 20070142436, 20070123579, 20070112059, 20070105890, 20070098781, 20070093492, 20070082932, 20070082908, 20070072810, 20070072804, 20070072803, 20070060547, 20070049619, 20070049596, 20070021477, 20060293297, 20060281796, 20060281727, 20060276487, 20060276410, 20060270722, 20060270701, 20060270679, 20060264457, 20060264433, 20060264401, 20060264400, 20060258646, 20060258621, 20060247226, 20060229286, 20060217428, 20060211682, 20060205711, 20060205675, 20060173056, 20060154866, 20060142585, 20060135767, 20060135561, 20060135512, 20060116393, 20060111336, 20060111428, 20060079541, 20060074058, 20060074087, 20060069116, 20060058323, 20060052382, 20060046978, 20060040963, 20060039974, 20060014953, 20060014764, 20060004074, 20050059724, 20050059716, 20050043292, 20050038020, 20050032804, 20050272765, 20050272652, 20050261271, 20050260732, 20050260712, 20050245538, 20050234235, 20050233978, 20050234108, 20050222242, 20050222222, 20050222140, 20050215784, 20050215603, 20050209249, 20050209159, 20050203095, 20050203031, 20050203027, 20050192324, 20050187227, 20050176771, 20050171093, 20050164989, 20050143377, 20050143405, 20050137224, 20050131019, 20050130985, 20050130981, 20050113310, 20050107390, 20050107309, 20050096348, 20050090539, 20050075330, 20050070719, 20050070706, 20050070535, 20050070531, 20050070530, 20050065148, 20050065145, 20050065144, 20050043299, 20050043292, 20050032804, 20050026921, 20050004205, 20050004117, 20050032804, 20040259903, 20040259902, 20040259883, 20040259870, 20040259843, 20040254226, 20050254167, 20040242898, 20040242636, 20040242568, 20040242566, 20040236102, 20040235752, 20040229926, 20040229848, 20040229820, 20040209891, 20040186153, 20040180925, 20040176428, 20040176406, 20040171555, 20040171848, 20040167341, 20040167133, 20040152745, 20040147434, 20040138215, 20040138214, 20040121964, 20040116328, 20040110817, 20040106656, 20040106802, 20040106655, 20040097510, 20040087587, 20040082570, 20040082497, 20040077645, 20040072892, 20040063935, 20040034014, 20030232788, 20030225102, 20030216450, 20030216382, 20030199528, 20030195188, 20030166578, 20030162820, 20030149071, 20030134802, 20030130281, 20030130199, 20030125304, 20030119750, 20030119738, 20030105077, 20030100563, 20030092630, 20030087950, 20030078247, 20030060494, 20020198242, 20020198205, 20020183367, 20020165164, 20020161001, 20020110560, 20020103384, 20030096857, 20020071838, 20020065239, 20020061839, 20020049164, 20020019411, 20020006899, 20010020006, the contents of each of which are incorporated by reference herein in their entireties

Yet further examples of DPP-IV inhibitors can be found in Application Publication Nos. WO 07/054,577, WO 07/053,865, WO OS/116029, WO 05/087235, WO 05/082348, WO 05/082849, WO 05/079795, WO 05/075426, WO 05/072530, WO 05/063750, WO 05/058849, WO 05/049022, WO 05/047297, WO 05/044195, WO 05/042488, WO 05/042003, WO 05/040095, WO 05/037828, WO 05/037779, WO 05/034940, WO 05/033099, WO 05/032590, WO 05/030751, WO 05/030127, WO 05/026148, WO 05/025554, WO 05/023762, WO 05/020920, WO 05/19168, WO 05/12312, WO 05/12308, WO 05/12249, WO 05/11581, WO 05/09956, WO 05/03135, WO 05/00848, WO 05/00846, WO 04/112701, WO 04/111051, WO 04/111041, WO 04/110436, WO 04/110375, WO 04/108730, WO 04/104216, WO 04/104215, WO 04/103993, WO 04/103276, WO 04/99134, WO 04/96806, WO 04/92128, WO 04/87650, WO 04/87053, WO 04/85661, WO 04/85378, WO 04/76434, WO 04/76433, WO 04/71454, WO 04/69162, WO 04/67509, WO 04/64778, WO 04/58266, WO 04/52362, WO 04/52850, WO 04/50022, WO 04/50658, WO 04/48379, WO 04/46106, WO 04/43940, WO 04/41820, WO 04/41795, WO 04/37169, WO 04/37181, WO 04/33455, WO 04/32836, WO 04/20407, WO 04/18469, WO 04/18468, WO 04/18467, WO 04/14860, WO 04/09544, WO 04/07468, WO 04/07446, WO 04/04661, WO 04/00327, WO 03/106456, WO 03/104229, WO 03/101958, WO 03/101448, WO 03/99279, WO 03/95425, WO 03/84940, WO 03/82817, WO 03/80633, WO 03/74500, WO 03/72556, WO 03/72528, WO 03/68757, WO 03/68748, WO 03/57666, WO 03/57144, WO 03/55881, WO 03/45228, WO 03/40174, WO 03/38123, WO 03/37327, WO 03/35067, WO 03/35057, WO 03/24965, WO 03/24942, WO 03/22871, WO 03/15775, WO 03/04498, WO 03/04496, WO 03/02530, WO 03/02596, WO 03/02595, WO 03/02593, WO 03/02553, WO 03/02531, WO 03/00181, WO 03/00180, WO 03/00250, WO 02/83109, WO 02/83128, WO 02/76450, WO 02/68420, WO 02/62764, WO 02/55088, WO 02/51836, WO 02/38541, WO 02/34900, WO 02/30891, WO 02/30890, WO 02/14271, WO 02/02560, WO 01/97808, WO 01/96295, WO 01/81337, WO 01/81304, WO 01/68603, WO 01/55105, WO 01/52825, WO 01/34594, WO 00/71135, WO 00/69868, WO 00/56297, WO 00/56296, WO 00/34241, WO 00/23421, WO 00/10549, WO 99/67278, WO 99/62914, WO 99/61431, WO 99/56753, WO 99/25719, WO 99/16864, WO 98/50066, WO 98/50046, WO 98/19998, WO 98/18763, WO 97/40832, WO 95/29691, WO 95/15309, WO 93/10127, WO 93/08259, WO 91/16339, EP 1517907, EP 1513808, EP 1492777, EP 1490335, EP 1489088, EP 1480961, EP 1476435, EP 1476429, EP 1469873, EP 1465891, EP 1463727, EP 1461337, EP 1450794, EP 1446116, EP 1442049, EP 1441719, EP 1426366, EP 1412357, EP1406873, EP 1406872, EP 1406622, EP 1404675, EP 1399420, EP 1399471, EP 1399470, EP 1399469, EP 1399433, EP 1399154, EP 1385508, EP 1377288, EP 1355886, EP 1354882, EP 1338592, EP 1333025, EP 1304327, EP 1301187, EP 1296974, EP 1280797, EP 1282600, EP 1261586, EP 1258476, EP 1254113, EP 1248604, EP 1245568, EP 1215207, EP 1228061, EP 1137635, EP 1123272, EP 1104293, EP 1082314, EP 1050540, EP 1043328, EP 0995440, EP 0980249, EP 0975359, EP 0731789, EP 0641347, EP 0610317, EP 0528858, CA 2466870, CA 2433090, CA 2339537, CA 2289125, CA 2289124, CA 2123128, DD 296075, DE 19834591, DE 19828113, DE 19823831, DE 19616486, DE 10333935, DE 10327439, DE 10256264, DE 10251927, DE 10238477, DE 10238470, DE 10238243, DE 10143840, FR 2824825, FR 2822826, JP2005507261; JP 2005505531, JP 2005502624, JP 2005500321, JP 2005500308, JP2005023038, JP 2004536115, JP 2004535445, JP 2004535433, JP 2004534836, JP 2004534815, JP 2004532220, JP 2004530729, JP 2004525929, JP 2004525179, JP 2004522786, JP 2004521149, JP 2004503531, JP 2004315496, JP 2004244412, JP 2004043429, JP 2004035574, JP 2004026820, JP 2004026678, JP 2004002368, JP 2004002367, JP 2003535898, JP 2003535034, JP 2003531204, JP 2003531191, JP 2003531118, JP 2003524591, JP 2003520849, JP 2003327532, JP 2003300977, JP 2003238566, JP 2002531547, JP 2002527504, JP 2002517401, JP 2002516318, JP 2002363157, JP 2002356472, JP 2002356471, JP 2002265439, JP 2001510442, JP 2000511559, JP 2000327689, JP 2000191616, JP 1998182613, JP 1998081666, JP 1997509921, JP 1995501078, JP 1993508624, the contents of each of which are incorporated by reference herein in their entireties.

In certain embodiments, the DPP-IV inhibitor is a small molecule with a molecular weight of less than 1000, 700 or 500 Daltons, e.g., an organic molecule other than a nucleic acid, or a protein or peptide.

In certain embodiments, the DPP-IV inhibitor is a β-amino acid derivative, such as 3(R)-Amino-1-[3-(trifluoromethyl)-5,6,7,8-tetrahydro[1,2,4]triazolo[4,3-a-]pyrazin-7-yl]-4-(2,4,5-trifluorophenyl)butan-1-one (MK-0431; Januvia), or its pharmaceutical salt, hydrate or polymorph, which are described in detail in U.S. Pat. No. 6,699,871, EP 1412357, WO 03/04498, and US 2003100563, the contents of each of which are incorporated by reference herein in their entireties. In some embodiments, the DPP-IV inhibitor is sitagliptin. Sitagliptin is described as an orally active and selective DPP-IV inhibitor and was recently approved in the U.S. and in Europe for the treatment of diabetes alone or in combination with metformin or sulfonylurea or a PPARγ agonist. See U.S. Pat. No. 6,699,871, Kim et al., 2005, J. Med. Chem. 48:141-151, the contents of each of which are incorporated by reference herein in their entireties.

In certain embodiments, the DPP-IV inhibitor is cyanopyrrolidide, such as (1-[[3-hydroxy-1-adamantyl)amino]acetyl]-2-cyano-(S)-pyrrolidine (LAF237 or vildagliptin), 1-[2-[5-cyanopyridin-2-yl)amino]ethylamino]acetyl-2-cyano-(S)-pyrrolidine (NVP-DPP728), or (1S,3S,5S)-2-[2(S)-Amino-2-(3-hydroxyadamantan-1-yl)acetyl]-2-azabicyclo[-3.1.0]hexane-3-carbonitrile (saxagliptin or BMS-47718), which are disclosed in detail, for example, in U.S. Pat. Nos. 6,617,340, 6,432,969, 6,395,767, 6,166,063, 6,124,305, 6,110,949, 6,011,155, 6,107,317, WO 98/19998 and JP 2000511559, WO 00/34241, EP 1137635, and JP 2002531547, the contents of each of which are incorporated by reference herein in their entireties.

In some embodiments, the DPP-IV inhibitor is vildagliptin. In some embodiments, the DPP-IV inhibitor is NVP-DPP728. Vildagliptin and NVP-DPP728 are described as an orally active and selective DPP-IV inhibitor. See Villhauer et al, 2002, J Med Chem 45:2362-2365, Villhauer et al, 2003, J Med Chem 46:2774-2789, the contents of each of which are incorporated by reference herein in their entireties. Vildagliptin (LAF 237) is currently undergoing Phase III clinical trial in the United States. It is approved for use in Europe in combination in combination with metformin or sulfonylurea or a thiazolidinedione.

In certain embodiments, the DPP-IV inhibitor is saxagliptin. Saxagliptin is currently in Phase III clinical trail in the U.S. and Europe for the treatment of type II diabetes. See Augeri et al., 2005, J. Med. Chem. 48(5):5025-5037, the contents of which is incorporated by reference herein in its entirety.

In certain embodiments, the DPP-IV inhibitor is 3-(L-Isoleucyl)thiazolidine (isoleucine-thiazolidide or PSN-9301). Isoleucine-thiazolidide can be found in JP 2001510442, WO 97/40832, U.S. Pat. No. 6,303,661, and DE 19616486, the contents of each of which are incorporated by reference herein in their entireties. Isoleucine-thiazolidide is described as an orally active and selective DPP-IV inhibitor. See Pederson et al, 1998, Diabetes 47:1253-1258; Epstein et al., 2007, Curr. Opin. Investig. Drugs, 8(4):331-337, the contents of each of which are incorporated by reference herein in their entireties.

In certain embodiments, the DPP-IV inhibitor is SYR-322 (Alogliptin) or SYR-472 such as described in U.S. Pat. Nos. 7,169,926 and 7,034,039, the contents of each of which are incorporated by reference herein in their entireties.

In certain embodiments, the DPP-IV inhibitor is valine-pyrrolidide, such as disclosed in Deacon et al, Diabetes (1998) 47:764769; which is incorporated by reference herein in its entirety.

In certain embodiments, the DPP-IV inhibitor is [1-[2(S)-Amino-3-methylbutyryl]pyrrolidin-2(R)-yl]boronic acid (PT-100).

In certain embodiments, the DPP-IV inhibitor is β-phenethylamine, such as described in Nordhoff et al., 2006, Bioorganic Medical Chemistry Letters 16:1744-1748, is incorporated by reference herein in its entirety.

In certain embodiments, the DPP-IV inhibitor is PT-630 (DB-160), such as described in Application Publication No. WO 06/034435, which is incorporated by reference herein in its entirety.

In certain embodiments, the DPP-IV inhibitor is ABT-341, such as described in Pei et al., J. Med, Chem. 2006 Nov. 2; 49(22):6439-42, which is incorporated by reference herein in its entirety.

In certain embodiments, the DPP-IV inhibitor is ABT-279, such as described in Madar et al., J. Med. Chem. 2006 Oct. 19; 49(21):6416-20, which is incorporated by reference herein in its entirety.

In certain embodiments, the DPP-IV inhibitor is BI-1356/Ondero, such as described in Application Publication No. WO 04/18468, which is incorporated by reference herein in its entirety.

In certain embodiments, the DPP-IV inhibitor is SYR-619.

In certain embodiments, the DPP-IV inhibitor is GSK-823093.

In certain embodiments, the DPP-IV inhibitor is PSN 9301.

In certain embodiments, the DPP-IV inhibitor is TA-6666.

In certain embodiments, the DPP-IV inhibitor is CR-14023.

In certain embodiments, the DPP-IV inhibitor is CR-14025.

In certain embodiments, the DPP-IV inhibitor is CR-14240.

In certain embodiments, the DPP-IV inhibitor is CR-13651.

In certain embodiments, the DPP-IV inhibitor is NNC-72-2138.

In certain embodiments, the DPP-IV inhibitor is NN-7201.

In certain embodiments, the DPP-IV inhibitor is PHX-1149.

In certain embodiments, the DPP-IV inhibitor is PHX-1004.

In certain embodiments, the DPP-IV inhibitor is SNT-189379.

In certain embodiments, the DPP-IV inhibitor is GRC-8087.

In certain embodiments, the DPP-IV inhibitor is SK-0403.

In certain embodiments, the DPP-IV inhibitor is GSK-825964.

In certain embodiments, the DPP-IV inhibitor is TS-021.

In certain embodiments, the DPP-IV inhibitor is GRC-8200.

In certain embodiments, the DPP-IV inhibitor is GRC-8116.

In certain embodiments, the DPP-IV inhibitor is FE107542.

In certain embodiments, the DPP-IV inhibitor is MP-513.

In certain embodiments, the DPP-IV inhibitor is B1356.

In certain embodiments, the DPP-IV inhibitor is ALS 2-0426.

In certain embodiments, the DPP-IV inhibitor is ABT279.

In certain embodiments, the DPP-IV inhibitor is TS-201.

In certain embodiments, the DPP-IV inhibitor is KRP-104.

In certain embodiments, the DPP-IV inhibitor is RM579.

In certain embodiments, the DPP-IV inhibitor is LY2463665.

In certain embodiments, the DPP-IV inhibitor is ARI-2243. In certain embodiments, the DPP-IV inhibitor is SSR-162369.

5.3.2.3 Other Second Therapeutic Agents

In some embodiments the second therapeutic agent is an insulin receptor agonist. In some embodiments, the insulin receptor agonist is human insulin or insulin analog; basal insulin such as Lantus (insulin glargine), Levemir (insulin detemir), NN5401, NN-344, Albulin-G; or fast acting insulin such as Novolog (insulin aspart), Humalog (insulin lispro), Apidra (insulin glulisine).

In some embodiments, the second therapeutic agent is an amylin receptor agonist such as Symlin (pramlintide).

In some embodiments, the second therapeutic agent is glucose-dependent insulinotropic peptide/gastric inhibitory polypeptide (GIP) analog; glucagon receptor (GCGR) antagonist such as BAY-27-9955, Cpd G, or ISIS-325,568; glucocorticoid receptor (GCCR) antagonist such as ISIS-377,131; achromium and vanadium salt or derivative; 11beta-hydroxysteroid dehydrogenase (11beta-HSD1 and 11beta-HSD2) dehydrogenase and reductase inhibitor such as BVT-3498; a protein tyrosine phosphatase 1b (PTP 1b) inhibitor; glucose transporter (GLUT) and isoforms (GLUT1, GLUT4) inhibitor; sodium-glucose cotransporter and isoforms (SGLT1, SGLT2) inhibitor such as dapaglifozin, sergilfozin, and AVE-2268; sirtuin (SIRT) and isoforms agonist (SIRT1) such as resveratrol, SRT-501; a PPAR gamma/delta agonist; a PPAR alpha/gamma agonist such as tesaglitasar, muraglitazar, naveglitazar; a fructose-1,6-bisphosphatase (FBPase) inhibitor, such as CS-917, MB-7803; a glucose-dependent insulinotropic receptor (GDIR, G protein-coupled receptor 119, GPR-119) agonist such as ADP-668; a glucose-dependent insulin secretion by G protein-coupled receptors GPR-40, GPR-120, GPR-109A (HM-74A) agonist; fibroblast growth factor (FGF) and isoforms (FGF-21) analog; presenilins-associated rhomboid-like protein (PSARL) antagonist such as CXS-203; hepatic insulin sensitizing substance (HISS), bone morphogenic protein-9 (BMP-9); osteocalcin; visfatin (nicotinamide phosphoribosyltransferase, Nampt); selective PPAR gamma modulator (SPPARM) such as metaglidasen, MBX-2044; glucokinase (GK) activator such as RO-28-1675; glycogen phosphates (GP) inhibitor such as PSN-357; beta-cell growth factor such as islet neogenesis gene-associated protein (INGAP); CD-3 antagonist such as teplizumab, GAD65 antagonist such as Diamyd, DiaPep277, interleukin-1 inhibitor (IL-1) such as XOMA-052, jun N-terminal kinase (JNK) inhibitor, tolerogen such as NBI-6024, TRX4.

In some embodiments, the second therapeutic agent is an anti-obesity agent. In some embodiments, the anti-obesity agent is a cannabinoid 1 receptor (CB1R) inverse agonist and antagonist such as Acomplia/Zimulti (rimonabant), Meridia (Sibutramine), or Xenical (Orlistad).

In some embodiments, the second therapeutic agent is a gastro-intestinal hormone analog. In some embodiments, the gastro-intestinal hormone analog is a glucagon-like peptide-2 (GLP-2) analog such as Gattex (teduglutide); a peptide YY analog such as PYY(1-36), PYY(3-36); a pancreatic polypeptide (PP) analog; or a gastrin analog.

5.3.3 Selecting Subjects for Treatment

In one aspect, the present invention provides methods of selecting a subject for treatment with an insulinotropic peptide conjugate or formulation provided herein, comprising identifying a subject that has been previously treated with an anti-diabetic agent. Previous treatments with any antidiabetic agent known in the art can serve as a basis for identifying a subject for treatment with an insulinotropic peptide conjugate, e.g., an insulinotropic peptide conjugate described herein. Exemplary anti-diabetic agents are provided above. In some embodiments, the anti-diabetic agent is an oral anti-diabetic agent (OAD). In some embodiments, the subject is identified for treatment if the subject has not been previously treated with an antidiabetic agent, e.g., an OAD. In other embodiments, the subject is identified for treatment if the subject has previously been treated with an anti-diabetic agent, e.g., an OAD. Whether a subject has been previously treated with an anti-diabetic agent, e.g., an OAD, can be determined according to the judgment of the practitioner in the art. In certain embodiments, the present invention provides methods of selecting a subject for treatment with an insulinotropic peptide conjugate or formulation provided herein, comprising identifying a subject that has experienced hypoglycemia with the other anti-diabetic agent.

In certain embodiments, the present invention provides methods of selecting a subject for treatment with an insulinotropic peptide conjugate or formulation provided herein, comprising identifying a subject that has undergone previous treatment and experienced weight gain or undesirable weight gain.

In certain embodiments, the present invention provides methods of selecting a subject for treatment with an insulinotropic peptide conjugate or formulation provided herein, comprising identifying a subject that has been previously treated with a second active agent, e.g., an OAD such as sulfonylurea, metformin or a thiazolidinedione, the method can further comprise determining whether administration of the anti-diabetic agent resulted in a desirable therapeutic outcome, for example, acceptable control of the subject's glucose levels as determined by a practitioner of skill in the art. Acceptable glycemic control can be indicated by, but limited to, a decrease in whole blood glucose, a decrease in plasma blood glucose, a decrease in interstitial glucose (IG), and/or a decrease in HbA1c levels. In some embodiments, the present invention provides methods of selecting a subject for treatment with an insulinotropic peptide conjugate or formulation provided herein, comprising identifying a subject that has previously been administered an anti-diabetic agent, e.g., an OAD, e.g., resulting in acceptable control of the subject's glucose levels. In a particular embodiment, the present invention provides methods of selecting a subject for treatment with an insulinotropic peptide conjugate or formulation provided herein, comprising identifying a subject that has previously been administered an anti-diabetic agent, e.g., an OAD, not resulting in acceptable control of the subject's glucose levels. The foregoing methods can further comprise administering to the identified subject the insulinotropic peptide conjugate or formulation.

In some embodiments, the present invention provides methods of selecting a subject for treatment with an insulinotropic peptide conjugate or formulation provided herein, comprising identifying a subject that has been administered an antidiabetic agent, e.g., an OAD, prior to the first administration of the insulinotropic peptide conjugate. In a particular embodiment, the OAD is metformin. In some embodiments, the present invention provides methods of selecting a subject for treatment with an insulinotropic peptide conjugate or formulation provided herein, comprising identifying a subject that has been administered another antidiabetic agent, e.g., an OAD, not more than 30, 25, 20, 15, 10 or 5 days ago (as measured from the time of the identifying), said method further comprising administering the insulinotropic peptide conjugate or formulation within 30, 25, 20, 15, 10 or 5 days of the administration of the other antidiabetic agent. In a particular embodiment, the present invention provides methods of selecting a subject for treatment with an insulinotropic peptide conjugate or formulation provided herein, comprising identifying a subject that has not been administered an effective amount of another antidiabetic agent, e.g., an OAD, and then administering the other antidiabetic agent at the time (e.g. within the same hour or the same day as) of the first administration of the insulinotropic peptide conjugate. In other embodiments, the present invention provides methods of selecting a subject for treatment with an insulinotropic peptide conjugate or formulation provided herein, comprising identifying a subject that has not been administered an effective amount of another antidiabetic agent, e.g., an OAD, and then administering to the subject a first administration of the insulinotropic peptide conjugate or formulation.

In another aspect, the present invention provides methods for treating a subject having pre-diabetes, e.g., impaired glucose tolerance (IGT) and/or impaired fasting glucose (IFG), comprising administering to said subject an insulinotropic peptide conjugate, e.g., an insulinotropic peptide conjugate formulation described herein, in an amount effective to treat pre-diabetes. In some embodiments, the insulinotropic peptide conjugate is exendin-4(1-39) Lys⁴⁰ (ε-AEEA-MPA)-NH₂ conjugated to albumin. In some embodiments, the present invention provides methods of selecting a subject for treatment with an insulinotropic peptide conjugate or formulation provided herein, comprising identifying a subject that has IFG and/or IGT. In some embodiments, the methods comprise identifying a subject that has a diagnosis of IFG by a practitioner in the art. In some embodiments, the present invention provides methods of selecting a subject for treatment with an insulinotropic peptide conjugate or formulation provided herein, comprising identifying a subject that has that has fasting plasma glucose levels of >100 mg/dl (5.6 mmol/l) but <126 mg/dl (7.0 mmol/l). In other embodiments, the present invention provides methods of selecting a subject for treatment with an insulinotropic peptide conjugate or formulation provided herein, comprising identifying a subject that has that has a diagnosis of IGT by a practitioner in the art. In some embodiments, the methods comprise identifying a subject that has 2-hour oral glucose tolerance test levels of >140 mg/dl (7.8 mmol/l) but <200 mg/dl (11.1 mmol/l). The foregoing methods can further comprise administering to the identified subject the insulinotropic peptide conjugate or formulation.

In another aspect, the present invention provides methods for treating a subject who is obese but neither diabetic nor pre-diabetic, comprising administering to said subject an insulinotropic peptide conjugate, e.g. an insulinotropic peptide conjugate formulation described herein, in an amount effective to treat obesity. In some embodiments, the insulinotropic peptide conjugate is exendin-4(1-39) Lys⁴⁰ (ε-AEEA-MPA)-NH₂ conjugated to albumin. In some embodiments, the present invention provides methods of selecting a subject for treatment with an insulinotropic peptide conjugate or formulation provided herein, comprising identifying a subject that is obese but neither diabetic nor pre-diabetic for treatment with an insulinotropic peptide conjugate, wherein the methods comprise identifying a subject that has been previously treated with an anti-obesity agent. Previous treatments with any anti-obesity agent known in the art can serve as a basis for selection of a subject for treatment with an insulinotropic peptide conjugate, e.g. an insulinotropic peptide conjugate described herein. In some embodiments, the anti-obesity agent is Orlistat. In some embodiments, the anti-obesity agent is Sibutramine. In other embodiments, the anti-obesity agent is Liraglutide (NN2211). Liraglutide (NN2211) is a GLP-1 analog having the structure Arg(34)Lys(26)-(N-epsilon-(gamma-Glu(N-alpha-hexadecanoyl))-GLP-1(7-36)-NH₂. In some embodiments, the subject is selected for treatment if the subject has not been previously treated with Liraglutide. In other embodiments, the present invention provides methods of selecting a subject for treatment with an insulinotropic peptide conjugate or formulation provided herein, comprising identifying a subject that has previously been treated with Liraglutide. The foregoing methods can further comprise administering to the identified subject the insulinotropic peptide conjugate or formulation.

In certain embodiments, where the subject has been previously treated with Liraglutide, the present invention provides methods of selecting a subject for treatment with an insulinotropic peptide conjugate or formulation provided herein, comprising identifying a subject that has previously been administered Liraglutide resulting in a desirable therapeutic outcome, for example, weight loss amounting to greater than 5% of the subject's baseline weight, as determined by a practitioner of skill. In some embodiments, the present invention provides methods of selecting a subject for treatment with an insulinotropic peptide conjugate or formulation provided herein, comprising identifying a subject that has previously been administered Liraglutide resulting in weight loss amounting to greater than 5% of the subject's baseline weight. In a particular embodiment, the present invention provides methods of selecting a subject for treatment with an insulinotropic peptide conjugate or formulation provided herein, comprising identifying a subject that has previously been administered Liraglutide not resulting in weight loss amounting to greater than 5% of the subject's baseline weight. The foregoing methods can further comprise administering to the identified subject the insulinotropic peptide conjugate or formulation.

5.3.4 Treatment of Nervous System Disorders

The insulinotropic peptide conjugates and formulations provided herein provided herein can be used as a sedative. In one aspect of the invention, there is provided a method of sedating a mammalian subject having an abnormality resulting in increased activation of the central or peripheral nervous system. The method comprises administering a pharmaceutical formulation comprising an insulinotropic peptide conjugate described herein to the subject in an amount sufficient to produce a sedative or anxiolytic effect on the subject. The pharmaceutical formulation can be administered intracerebroventricularly, orally, subcutaneously, intramuscularly, or intravenously. Such methods are useful to treat or ameliorate nervous system conditions such as anxiety, movement disorder, aggression, psychosis, seizures, panic attacks, hysteria and sleep disorders.

In a related aspect, the invention encompasses a method of increasing the activity of a mammalian subject, comprising administering a pharmaceutical formulation comprising an insulinotropic peptide conjugate described herein to the subject in an amount sufficient to produce an activating effect on the subject. Preferably, the subject has a condition resulting in decreased activation of the central or peripheral nervous system. The pharmaceutical formulations can be used in the treatment of an insulinotropic peptide-related disease or condition. In certain embodiments, the pharmaceutical formulations can be used in the treatment or amelioration of depression, schizoaffective disorders, sleep apnea, attention deficit syndromes with poor concentration, memory loss, forgetfulness, and narcolepsy, to name just a few conditions in which arousal of the central nervous system may be advantageous.

The insulinotropic peptide conjugates and formulations provided herein provided herein can also be used to induce arousal for the treatment or amelioration of depression, schizoaffective disorders, sleep apnea, attention deficit syndromes with poor concentration, memory loss, forgetfulness, and narcolepsy. The therapeutic efficacy of the treatment can be monitored by subject interview to assess their condition, by psychological/neurological testing, or by amelioration of the symptoms associated with these conditions. For example, treatment of narcolepsy can be assessed by monitoring the occurrence of narcoleptic attacks. As another example, effects of modified ITPs on the ability of a subject to concentrate, or on memory capacity, can be tested using any of a number of diagnostic tests well known to those of skill in art.

5.3.5 Post Surgery Treatment

The insulinotropic peptide conjugates and formulations provided herein provided herein can be utilized for post surgery treatments. A subject is in need of a pharmaceutical formulation comprising a conjugated insulinotropic peptide described herein for about 1-16 hours before surgery is performed on the subject, during surgery on the subject, and after the subject's surgery for a period of not more than about 5 days.

The pharmaceutical formulations are administered from about sixteen hours to about one hour before surgery begins. The length of time before surgery when the compounds used in the present invention should be administered in order to reduce catabolic effects and insulin resistance is dependent on a number of factors. These factors are generally known to the physician of ordinary skill, and include, most importantly, whether the subject is fasted or supplied with a glucose infusion or beverage, or some other form of sustenance during the preparatory period before surgery. Other important factors include the subject's sex, weight and age, the severity of any inability to regulate blood glucose, the underlying causes of any inability to regulate blood glucose, the expected severity of the trauma caused by the surgery, the route of administration and bioavailability, the persistence in the body, the formulation, and the potency of the compound administered. A preferred time interval within which to begin administration of the modified insulinotropic peptides used in the present invention is from about one hour to about ten hours before surgery begins. The most preferred interval to begin administration is between two hours and eight hours before surgery begins.

Insulin resistance following a particular type of surgery, elective abdominal surgery, is most profound on the first post-operative day, lasts at least five days, and may take up to three weeks to normalize. Thus, the post-operative subject may be in need of administration of the pharmaceutical formulations of the present invention for a period of time following the trauma of surgery that will depend on factors that the physician of ordinary skill will comprehend and determine. Among these factors are whether the subject is fasted or supplied with a glucose infusion or beverage, or some other form of sustenance following surgery, and also, without limitation, the subject's sex, weight and age, the severity of any inability to regulate blood glucose, the underlying causes of any inability to regulate blood glucose, the actual severity of the trauma caused by the surgery, the route of administration and bioavailability, the persistence in the body, the formulation, and the potency of the compound administered. The preferred duration of administration of the compounds used in the present invention is not more than five days following surgery.

5.3.6 Insulin Resistance Treatment

The insulinotropic peptide conjugates and formulations provided herein provided herein can be utilized to treat insulin resistance independently from their use in post surgery treatment. Insulin resistance may be due to a decrease in binding of insulin to cell-surface receptors, or to alterations in intracellular metabolism. The first type, characterized as a decrease in insulin sensitivity, can typically be overcome by increased insulin concentration. The second type, characterized as a decrease in insulin responsiveness, cannot be overcome by large quantities of insulin. Insulin resistance following trauma can be overcome by doses of insulin that are proportional to the degree of insulin resistance, and thus is apparently caused by a decrease in insulin sensitivity.

5.3.7 Treatment of Diabetes or Obesity with Reduced Nausea

The insulinotropic peptide conjugates and formulations provided herein can be used in the treatment of an insulinotropic peptide related disease or condition while reducing nausea side effect such as described in U.S. patent application Ser. No. 11/595,576 (Publication No. 2007/0207958), entitled “Method of Treatment of Diabetes and/or Obesity with Reduced Nausea Effect,” filed Nov. 9, 2006, which is incorporated by reference herein in its entirety.

5.3.8 Other conditions

The insulinotropic peptide conjugates and formulations provided herein can be used to alter the concentration of fibrinogen in a subject in need thereof. Provided herein are methods of decreasing the concentration of fibrinogen in a subject in need thereof, the method comprising administering to the subject an effective amount of an insulinotropic peptide conjugate or formulation provided herein, wherein the concentration of fibrinogen is decreased in the subject. Provided herein are methods of decreasing the concentration of fibrinogen in a subject with an elevated level of fibrinogen, the methods comprising administering to the subject an effective amount of an insulinotropic peptide conjugate or formulation provided herein, wherein the concentration of fibrinogen is decreased in the subject. Provided herein are methods of providing an improved cardiovascular risk profile of a subject in need thereof comprising administering to the subject an effective amount of an insulinotropic peptide conjugate or formulation provided herein and measuring a decrease in concentration of fibrinogen in the subject, wherein the cardiovascular risk profile of the subject is improved. Provided herein are methods of providing an improved cardiovascular risk profile of a subject with an elevated level of fibrinogen comprising administering to the subject an effective amount of an insulinotropic peptide conjugate or formulation provided herein and measuring a decrease in the concentration of fibrinogen in the subject, wherein the cardiovascular risk profile of the subject is improved. Provided herein are methods of treating a subject in need thereof, comprising administering to the subject an effective amount of an insulinotropic peptide conjugate or formulation provided herein, wherein the concentration of fibrinogen in the subject is decreased. Provided herein are methods of treating a subject with an elevated level of fibrinogen, comprising administering to the subject an effective amount of an insulinotropic peptide conjugate or formulation provided herein, wherein the concentration of fibrinogen in the subject is decreased.

The insulinotropic peptide conjugates and formulations provided herein can be used to alter the lipoprotein particle size or subclass composition in a subject in need thereof. Provided herein are methods for increasing the concentration of large LDL, large HDL, total HDL or any combination of said lipoproteins in a subject in need thereof comprising administering to said subject an effective amount of an insulinotropic peptide conjugate or formulation provided herein, wherein the concentration of large LDL, large HDL, total HDL, or any combination of said lipoproteins is increased in said subject. Provided herein are methods for increasing the concentration of large LDL, large HDL, total HDL or any combination of said lipoproteins in a subject who has a decreased large LDL, large HDL, total HDL level, or any combination thereof comprising administering to said subject a an effective amount of an insulinotropic peptide conjugate or formulation provided herein, wherein the concentration of large LDL, large HDL, total HDL, or any combination of said lipoproteins is increased in said subject. Provided herein are methods for decreasing the concentration of small LDL, very small LDL, total LDL or any combination of said lipoproteins in a subject in need thereof comprising administering to said subject an effective amount of an insulinotropic peptide conjugate or formulation provided herein, wherein the concentration of small LDL is decreased. Provided herein are methods for decreasing the concentration of small LDL, very small LDL, total LDL or any combination of said lipoproteins in a subject who has an elevated level of small LDL, very small LDL, total LDL or any combination thereof comprising administering to said subject an effective amount of an insulinotropic peptide conjugate or formulation provided herein, wherein the concentration of small LDL is decreased. Provided herein are methods for providing an improved cardiovascular risk profile of a subject in need thereof comprising administering to an effective amount of an insulinotropic peptide conjugate or formulation provided herein and measuring an increased concentration of large LDL, large HDL, total HDL or any combination of said lipoproteins, wherein the cardiovascular risk profile of said subject is improved. Provided herein are methods for providing an improved cardiovascular risk profile of a subject who has a decreased level of large LDL, large HDL, total HDL or any combination thereof comprising administering to said subject an effective amount of an insulinotropic peptide conjugate or formulation provided herein and measuring an increased concentration of large LDL, large HDL, total HDL or any combination of said lipoproteins, wherein the cardiovascular risk profile of said subject is improved. Provided herein are methods for treating a subject with an elevated level of small LDL, very small LDL or total LDL or any combination of said lipoproteins, comprising administering to said subject an effective amount of an insulinotropic peptide conjugate or formulation provided herein, wherein the concentration of small LDL, very small LDL, total LDL or any combination of said lipoproteins is decreased in said subject. Provided herein are methods for increasing the average particle size of LDL or HDL in a subject in need thereof comprising administering to said subject an effective amount of an insulinotropic peptide conjugate or formulation provided herein, wherein the particle size of LDL or HDL is increased in said subject. Provided herein are methods for increasing the average particle size of LDL or HDL in a subject who has an elevated level of small LDL, a decreased level of large HDL, a decreased level of total HDL or any combination thereof comprising administering to said subject a an effective amount of an insulinotropic peptide conjugate or formulation provided herein, wherein the particle size of LDL or HDL is increased in said subject.

5.3.9 Dosage and Frequency of Administration

The insulinotropic peptide conjugates, e.g., insulintropic peptide conjugate formulations, can be administered according to any technique deemed suitable by one of skill in the art. For example, the insulinotropic peptide conjugates, e.g., insulinotropic peptide conjugate formulations, can be administered by any of the following means: (a) enterally, e.g., orally (by mouth), rectally (e.g., in the form of a suppository or an enema), by feeding tube (e.g., gastric feeding tube, duodenal feeding tube, gastrostromy); (b) parenterally, e.g., subcutaneously, intravenously, intramuscularly, intradermally (into the skin itself), transdermally (diffusion through skin, e.g., intact skin), intra-arterially, intra-peritoneally, intracardiac (into the heart) administration, intraosseous (into the bone marrow) administration intrathecally (into the spinal canal), transmucosally (diffusion through a mucous membrane, e.g., insufflation (snorting), nasally, e.g., intranasally), sublingually (under the tongue), buccally (through the cheek), vaginally, by inhalation (e.g., pulmonary administration); (c) topically; (d) epidurally (injection or infusion into the epidural space); and (e) intravitreally. Each administration of insulinotropic peptide conjugates, e.g., insulinotropic peptide conjugate formulations, can be by bolus or by infusion. In preferred embodiments, the insulinotropic peptide conjugate, e.g., insulinotropic peptide conjugate formulation, is administered subcutaneously. In a particular embodiment, the insulinotropic peptide conjugate, e.g., insulinotropic peptide conjugate formulation, is administered subcutaneously using a needle, e.g., a 25-gauge needle, a 26-gauge needle, a 27-gauge needle, a 28-gauge needle, a 29-gauge needle, a 30-gauge needle, a 31-gauge needle, a 32-gauge needle, or a 33-gauge needle, or a higher gauge needle.

The dosage and frequency of administration of the insulinotropic peptide conjugates, e.g., insulinotropic peptide conjugate formulations, can be determined by one skilled in the art. The amount of an insulinotropic peptide conjugate that will be effective in the treatment of a disorder or condition will vary with the nature and severity of the disorder or condition, and the route by which the active ingredient is administered. The frequency and dosage will also vary according to factors specific for each subject depending on the severity of the disorder or condition, the route of administration, as well as age, body weight, response, and the past medical history of the subject. Effective doses may be extrapolated from dose-response curves derived from in vitro or animal model test systems.

Exemplary doses of an insulinotropic peptide conjugate include milligram or microgram amounts of the insulinotropic peptide conjugate per kilogram of subject or sample weight (e.g., about 1 microgram per kilogram to about 50 microgram per kilogram, e.g., about 10 microgram per kilogram to about 30 microgram per kilogram).

In some embodiments, the dosage of insulinotropic peptide conjugate, e.g., insulinotropic peptide conjugate formulation, which may be effective to achieve the desired therapeutic response for a particular subject is administered to the subject in accordance with a weekly dosing regime administered over a number of weeks. In some embodiments, the insulinotropic peptide conjugate, e.g., insulinotropic peptide conjugate formulation, can be administered once a week (e.g., as a single dose). In some embodiments, the insulinotropic peptide conjugate, e.g., insulinotropic peptide conjugate formulation, can be administered twice a week (e.g., as two of the same or different doses). In other embodiments, the insulinotropic peptide conjugate, e.g., insulinotropic peptide conjugate formulation, can be administered once every 2, 3, 4, 5 or 6 days. In other embodiments, the insulinotropic peptide conjugate, e.g., insulinotropic peptide conjugate formulation, can be administered once every 8, 9, 10, 11, 12 or 13 days. In other embodiments, the insulinotropic peptide conjugate, e.g., insulinotropic peptide conjugate formulation, can be administered two times every 3, 4, 5, 6, 7 or 8 day period. In other embodiments, the insulinotropic peptide conjugate, e.g., insulinotropic peptide conjugate formulation, can be administered two times every 9, 10, 11, 12, 13 or 14 day period.

In some embodiments, the dose is administered once a week or twice a week and the dose comprises the insulinotropic peptide conjugate in an amount between about 1000 μg and 3000 μg (e.g., 1025 μg, 1050 μg, 1075 μg, 1100 μg, 1125 μg, 1150 μg, 1175 μg, 1200 μg, 1225 μg, 1250 μg, 1275 μg, 1300 μg, 1325 μg, 1350 μg, 1375 μg, 1400 μg, 1425 μg, 1450 μg, 1475 μg, 1500 μg, 1525 μg, 1550 μg, 1575 μg, 1600 μg, 1625 μg, 1650 μg, 1675 μg, 1700 μg, 1725 μg, 1750 μg, 1775 μg, 1800 μg, 1825 μg, 1850 μg, 1875 μg, 1900 μg, 1925 μg, 1950 μg, 1975 μg, 2000 μg, 2025 μg, 2050 μg, 2075 μg, 2100 μg, 2125 μg, 2150 μg, 2175 μg, 2200 μg, 2225 μg, 2250 μg, 2275 μg, 2300 μg, 2325 μg, 2350 μg, 2375 μg, 2400 μg, 2425 μg, 2450 μg, 2475 μg, 2500 μg, 2525 μg, 2550 μg, 2575 μg, 2600 μg, 2625 μg, 2650 μg, 2675 μg, 2700 μg, 2725 μg, 2750 μg, 2775 μg, 2800 μg, 2825 μg, 2850 μg, 2875 μg, 2900 μg, 2925 μg, 2950 μg, or 2975 μg), preferably between about 1000 μg and 2750 μg (e.g., 1025 μg, 1050 μg, 1075 μg, 1100 μg, 1125 μg, 110 μg, 1175 μg, 1200 μg, 1225 μg, 1250 μg, 1275 μg, 1300 μg, 1325 μg, 1350 μg, 1375 μg, 1400 μg, 1425 μg, 1450 μg, 1475 μg, 1500 μg, 1525 μg, 1550 μg, 1575 μg, 1600 μg, 1625 μg, 1650 μg, 1675 μg, 1700 μg, 1725 μg, 1750 μg, 1775 μg, 1800 μg, 1825 μg, 1850 μg, 1875 μg, 1900 μg, 1925 μg, 1950 μg, 1975 μg, 2000 μg, 2025 μg, 2050 μg, 2075 μg, 2100 μg, 2125 μg, 2150 μg, 2175 μg, 2200 μg, 2225 μg, 2250 μg, 2275 μg, 2300 μg, 2325 μg, 2350 μg, 2375 μg, 2400 μg, 2425 μg, 2450 μg, 2475 μg, 2500 μg, 2525 μg, 2550 μg, 2575 μg, 2600 μg, 2625 μg, 2650 μg, 2675 μg, 2700 μg, or 2725 μg), and more preferably between about 1000 and 2500 μg (e.g., 1025 μg, 10501 g, 1075 μg, 1100 μg, 1125 μg, 1150 μg, 1175 μg, 1200 μg, 1225 μg, 1250 μg, 1275 μg, 1300 μg, 1325 μg, 1350 μg, 1375 μg, 1400 μg, 1425 μg, 1450 μg, 1475 μg, 1500 μg, 1525 μg, 1550 μg, 1575 μg, 1600 μg, 1625 μg, 1650 μg, 1675 μg, 1700 μg, 1725 μg, 1750 μg, 1775 μg, 1800 μg, 1825 μg, 1850 μg, 1875 μg, 1900 μg, 1925 μg, 1950 μg, 1975 μg, 2000 μg, 2025 μg, 2050 μg, 2075 μg, 2100 μg, 2125 μg, 2150 μg, 2175 μg, 2200 μg, 2225 μg, 2250 μg, 2275 μg, 2300 μg, 2325 μg, 2350 μg, 2375 μg, 2400 μg, 2425 μg, 2450 μg, or 2475 μg), most preferably between about 1000 μg to 2000 μg (e.g., 1025 μg, 1050 μg, 1075 μg, 1100 μg, 1125 μg, 1150 μg, 1175 μg, 1200 μg, 1225 μg, 1250 μg, 1275 μg, 1300 μg, 1325 μg, 1350 μg, 1375 μg, 1400 μg, 1425 μg, 1450 μg, 1475 μg, 1500 μg, 1525 μg, 1550 μg, 1575 μg, 1600 μg, 1625 μg, 1650 μg, 1675 μg, 1700 μg, 1725 μg, 1750 μg, 1775 μg, 1800 μg, 1825 μg, 1850 μg, 1875 μg, 1900 μg, 1925 μg, 1950 μg, or 1975 μg) of the insulinotropic peptide conjugate. In some embodiments, the dose comprises the insulinotropic peptide in an amount between 1000 μg to 2000 μg. In some embodiments, the dose comprises the insulinotropic peptide in an amount between 1500 μg to 2000 μg.

In certain embodiments, the total weekly dose is administered in a single administration during the week, i.e., once a week, and the total weekly dose comprises the insulinotropic peptide conjugate in an amount of 1000 μg or 1500 μg. In certain embodiments, the total weekly dose is administered once a week, and the dose comprises the insulinotropic peptide conjugate in an amount of 2000 μg. In certain embodiments, the total weekly dose is administered over two administrations during the week, i.e., twice a week, and each administration comprises the insulinotropic peptide conjugate in an amount of 1000 μg, amounting to a total weekly dose of 2000 μg. In certain embodiments, the total weekly dose is administered twice a week, and each administration comprises the insulinotropic peptide conjugate in an amount of 1500 μg, amounting to a total weekly dose of 3000 μg. In certain embodiments, the total weekly dose is administered twice a week, and each administration comprises the insulinotropic peptide conjugate in an amount of 1600 μg, amounting to a total weekly dose of 3200 μg. In certain embodiments, the total weekly dose is administered twice a week, and each administration comprises the insulinotropic peptide conjugate in an amount of 1700 μg, amounting to a total weekly dose of 3400 μg. In certain embodiments, the total weekly dose is administered twice a week, wherein the first administration comprises the insulinotropic peptide conjugate in an amount of 1500 μg and the second administration comprises the insulinotropic peptide in an amount of 2000 μg, amounting to a total weekly dose of 3500 μg. In certain embodiments, the total weekly dose is administered twice a week, and each administration comprises the insulinotropic peptide conjugate in an amount of 1750 μg, amounting to a total weekly dose of 3500 μg. In certain embodiments, the total weekly dose is administered twice a week, and each administration comprises the insulinotropic peptide conjugate in an amount of 1800 μg, amounting to a total weekly dose of 3600 μg. In certain embodiments, the total weekly dose is administered twice a week, and each administration comprises the insulinotropic peptide conjugate in an amount of 1900 μg, amounting to a total weekly dose of 3800 μg. In certain embodiments, the total weekly dose is administered twice a week, and each administration comprises the insulinotropic peptide conjugate in an amount of 2000 μg, amounting to a total weekly dose of 4000 μg.

In certain embodiments, these dosages, or other exemplary dosages described herein, can be provided in a delivery device for convenient administration of the dose to the subject. Any delivery device known in the art can be used. In particular embodiments, the delivery device is a syringe configured for subcutaneous delivery, e.g. a 0.3, 0.5, 1, 2, 3 or greater than 3 ml syringe having a 25, 26, 27, 28, 29, 30, 31, 32, 33, or larger than 33-gauge needle.

Different therapeutically effective amounts of the insulinotropic peptide conjugate may be applicable for different disorders and conditions, as will be readily known by those of ordinary skill in the art.

In certain embodiments, administration of the insulinotropic peptide conjugate, e.g., insulinotropic peptide conjugate formulations, provided herein can be repeated, and the administrations can be separated by at least 12 hours, one day, 36 hours, two days, 60 hours, three days, 84 hours, four days, five days, six days, 7 days, 8 days, 9 days, 10 days, 11 days, 12 days, 13 days, 14 days, 15 days, 16 days, 17 days, 18 days, 19 days, 20 days, 21 days, 4 weeks, 6 weeks, 2 months, 75 days, 3 months, or 6 months. In certain embodiments, the repeated administration of the insulinotropic peptide conjugate, e.g., insulinotropic peptide conjugate formulation, is separated by three or four days, by one week, or by two weeks.

In certain embodiments, the methods can be practiced, and the formulations can be given, as a single, one time dose, or chronically. By chronic/chronically is meant that the formulations of the invention are administered more than once to a given individual. For example, chronic administration can be multiple doses of a formulation administered to a subject, on a weekly basis, a biweekly basis, monthly basis, or more or less frequently, as will be apparent to those of skill in the art. Chronic administration can continue for weeks, months, or years if appropriate according to the judgment of the practitioner of skill in the art. Furthermore, if certain doses, in the judgment of the practioner of skill in the art, show tolerability profiles which may not be acceptable, e.g., frequent and severe bouts of nausea and vomiting, the practioner can reduce the dose to reduce such profiles. For example, the dose as described herein can be reduced from a 1500 μg dose to a 1000 μg dose or a 2000 μg dose can be reduced to a 1500 μg dose.

The dose of insulinotropic peptide conjugate administered over the course of repeated administrations can be held constant, or can be varied, e.g., increased or decreased, relative to the dose of insulinotropic peptide conjugate administered in earlier administrations. In certain embodiments, the dose of insulinotropic peptide conjugate administered over the course of repeated administrations is held constant. Thus, in some embodiments, a weekly dose of 1500 μg of insulinotropic peptide conjugate is administered to the subject, and administration is repeated on a weekly basis at 1500 μg per week. In other embodiments, a weekly dose of 3000 μg of insulinotropic peptide conjugate, delivered in two doses of 1500 μg, is administered to the subject, and twice-a-week administration is repeated on a weekly basis at a total weekly dose of 3000 μg of insulinotropic peptide conjugate per week. In some embodiments, a weekly dose of 2000 μg of insulinotropic peptide conjugate is administered to the subject, and administration is repeated on a weekly basis at 2000 μg per week. In other embodiments, a weekly dose of 4000 μg of insulinotropic peptide conjugate, delivered in two doses of 2000 μg, is administered to the subject, and twice-a-week administration is repeated on a weekly basis at a total weekly dose of 4000 μg of insulinotropic peptide conjugate per week. In some embodiments, a weekly dose of 3000 μg of insulinotropic peptide conjugate is administered to the subject, and administration is repeated on a weekly basis at 3000 μg per week.

In other embodiments, the dose of insulinotropic peptide conjugate, e.g., insulinotropic peptide conjugate formulation, administered to the subject is increased over the course of repeated administrations. For instance, in a particular embodiment, an initial total weekly dose of 1500 μg of insulinotropic peptide conjugate is administered to a subject for a first period of time, followed by administration of a total weekly dose of 2000 μg of insulinotropic peptide conjugate for a second period of time. In some embodiments, the first period of time is 1, 2, 3, 4, 5, 6, 7, 8 or more weeks. In a particular embodiment, the first period of time is four weeks, i.e., the increase in dose begins at the outset of the fifth week of dosing. In some embodiments, the second period of time is 1, 2, 3, 4, 5, 6, 7, 8 or more weeks. In a particular embodiment, the weekly dose is chronically administered (i.e, the second period of time is chronic administration as described herein). In another embodiment, an initial total weekly dose of 1500 μg of insulinotropic peptide conjugate is administered to a subject for four weeks, immediately followed by administration (starting at the fifth week) of a total weekly dose of 2000 μg of insulinotropic peptide conjugate chronically.

In a particular embodiment, the dose of insulinotropic peptide conjugate, e.g., insulinotropic peptide conjugate formulation, is administered to the subject in the following steps in the order stated: (a) administering 1.5 mg of the insulinotropic peptide conjugate to the subject once a week for a first duration of time; and (b) administering 2.0 mg of the insulinotropic peptide conjugate to the subject once a week for a second duration of time. In some embodiments, the first duration of time is 4 weeks. In some embodiments, the second duration of time is 8 weeks.

In another embodiment where the dose of insulinotropic peptide conjugate, e.g., insulinotropic peptide conjugate formulation, administered to the subject is increased over the course of repeated administrations, an initial total weekly dose of 3000 μg of insulinotropic peptide conjugate, delivered in two doses of 1500 μg, is administered to a subject for a first period of time, followed by administration of a total weekly dose of 4000 μg of insulinotropic peptide conjugate, delivered in two doses of 2000 μg, for a second period of time. In some embodiments, the first period of time is 1, 2, 3, 4, 5, 6, 7, 8 or more weeks. In a particular embodiment, the first period of time is four weeks, i.e., the increase in dose begins at the outset of the fifth week of dosing. In some embodiments, the second period of time is 1, 2, 3, 4, 5, 6, 7, 8 or more weeks. In a particular embodiment, the weekly dose is chronically administered (i.e, the second period of time is chronic administration as described herein). In another embodiment, an initial total weekly dose of 3000 μg of insulinotropic peptide conjugate, delivered in two doses of 1500 μg, is administered to a subject for four weeks, immediately followed by administration (starting at the fifth week) of a total weekly dose of 4000 μg of insulinotropic peptide conjugate, delivered in two doses of 2000 μg, chronically.

In a particular embodiment, the dose of insulinotropic peptide conjugate, e.g., insulinotropic peptide conjugate formulation, is administered to the subject in the following steps in the order stated: (a) administering 1.5 mg of the insulinotropic peptide conjugate to the subject twice a week for a first duration of time, and (b) administering 2.0 mg of the insulinotropic peptide conjugate to the subject twice a week for a second duration of time. In some embodiments, the first duration of time is 4 weeks. In some embodiments, the second duration of time is 8 weeks.

In another embodiment where the dose of insulinotropic peptide conjugate, e.g., insulinotropic peptide conjugate formulation, administered to the subject is increased over the course of repeated administrations, an initial total weekly dose of 1500 μg of insulinotropic peptide conjugate is administered to a subject for a first period of time, followed by administration of a total weekly dose of 2000 μg of insulinotropic peptide conjugate for a second period of time, followed by administration of a total weekly dose of 3000 μg of insulinotropic peptide conjugate for a third period of time. In some embodiments, the first period of time is 1, 2, 3, 4, 5, 6, 7, 8 or more weeks and the second period of time is 1, 2, 3, 4, 5, 6, 7, 8 or more weeks. In a particular embodiment, the first period of time is four weeks and the second period of time is four weeks, i.e., the increase in dose begins at the outset of the fifth and ninth week of dosing. In a particular embodiment, the first period of time is two weeks and the second period of time is two weeks, i.e., the increase in dose begins at the outset of the third and fifth week of dosing. In some embodiments, the third period of time is 1, 2, 3, 4, 5, 6, 7, 8 or more weeks. In a particular embodiment, the weekly dose is chronically administered (i.e, the third period of time is chronic administration as described herein). In another embodiment, an initial total weekly dose of 1500 μg of insulinotropic peptide conjugate is administered to a subject for four weeks, immediately followed by administration (starting at the fifth week) of a total weekly dose of 2000 μg of insulinotropic peptide conjugate for four weeks, immediately followed by administration (starting at the ninth week) of a total weekly dose of 3000 μg chronically. In another embodiment, an initial total weekly dose of 1500 Hg of insulinotropic peptide conjugate is administered to a subject for two weeks, immediately followed by administration (starting at the third week) of a total weekly dose of 2000 μg of insulinotropic peptide conjugate for two weeks, immediately followed by administration (starting at the fifth week) of a total weekly dose of 3000 μg chronically.

In a particular embodiment, the dose of insulinotropic peptide conjugate, e.g., insulinotropic peptide conjugate formulation, is administered to the subject in the following steps in the order stated: (a) administering 1.5 mg of the insulinotropic peptide conjugate to the subject once a week for a first duration of time; (b) administering 2.0 mg of the insulinotropic peptide conjugate to the subject once a week for a second duration of time; and (c) administering 3.0 mg of the insulinotropic peptide conjugate to the subject once a week for a third duration of time. In some embodiments, the first duration of time is 4 weeks. In some embodiments, the second duration of time is 8 weeks.

In other embodiments, the dose of insulinotropic peptide conjugate, e.g., insulinotropic peptide conjugate formulation, administered to the subject is decreased over the course of repeated administrations. For instance, in a particular embodiment, 1500 μg of insulinotropic peptide conjugate is administered twice a week for a total weekly dose of 3000 μg to a subject for a first period of time, followed by administration of a total weekly dose of 2000 μg of insulinotropic peptide conjugate for a second period of time. In another particular embodiment, 1500 μg of insulinotropic peptide conjugate is administered twice a week for a total weekly dose of 3000 μg to a subject for a first period of time, followed by administration of a 1000 μg of insulinotropic peptide conjugate twice a week for a total weekly dose of 2000 μg to the subject for a second period of time. In some embodiments, the first period of time is 1, 2, 3, 4, 5, 6, 7, 8 or more weeks. In a particular embodiment, the first period of time is four weeks. In some embodiments, the second period of time is 1, 2, 3, 4, 5, 6, 7, 8 or more weeks. In a particular embodiment, the weekly dose is chronically administered (i.e, the second period of time is chronic administration as described herein).

An effective amount of an insulinotropic peptide conjugate described herein will provide therapeutic benefit without causing substantial toxicity.

Toxicity of an insulinotropic peptide conjugate can be determined by standard pharmaceutical procedures in cell cultures or experimental animals, for example, by determining the LD50 (the dose lethal to 50% of the population) or the LD100 (the dose lethal to 100% of the population). The dose ratio between toxic and therapeutic effect is the therapeutic index. Compounds which exhibit high therapeutic indices are preferred. The data obtained from these cell culture assays and animal studies can be used in formulating a dosage range that is not toxic for use in human. The dosage of the compounds described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the subject's condition. (See, e.g., Fingl et al., 1996, In: The Pharmacological Basis of Therapeutics, 9th ed., Chapter 2, p. 29, Elliot M. Ross).

5.3.9.1 Routes of Administration and Dosage of Combination Therapies

The insulinotropic peptide conjugate, e.g., insulinotropic peptide conjugate formulation described herein and the one or more second therapeutic agents can be administered at essentially the same time, i.e., concurrently, e.g., within the same hour or same day, etc., or at separately staggered times, i.e. sequentially prior to or subsequent to the administration of the other anti-diabetic agent, e.g., on separate days, weeks, etc. The instant methods are therefore to be understood to include all such regimes of simultaneous or non-simultaneous treatment. In some embodiments, the insulinotropic peptide conjugate formulation is administered within 0.1, 0.5, 1, 2, 3, 4, 5, 6, 7, 8, 9, 10, 11, 12, 13, 14, 15, 16, 17, 18 or more than 18 hours of administration of the other second therapeutic agents. In some embodiments, the insulinotropic peptide conjugate formulation is administered within 1, 2, 3, 4, 5, 6, 7, 8, 9, 10, 11, 12, 13, 14 or more than 14 days of administration of the other second therapeutic agents. In some embodiments, the insulinotropic peptide conjugate formulation is administered within 1, 2, 3, 4, 5 or more than 5 weeks of administration of the second therapeutic agents.

In some embodiments of the combination therapies provided herein, the insulinotropic peptide conjugate formulation will be administered to the subject by subcutaneous injection in accordance with a dosing regime provided herein, e.g., at intervals of between 5, 6, 7, 8 or 9 days or at intervals of between 12, 13, 14, 15 or 16 days. Depending on the disease to be treated and the subject's condition, the particular one or more second therapeutic agents can be administered by oral, parenteral (e.g., intramuscular, intraperitoneal, intravenous, intracerebral ventricular (ICV), intracisternal injection or infusion, subcutaneous injection, or implant), inhalation spray, nasal, vaginal, rectal, sublingual, or topical routes of administration and can be formulated, alone or together, in suitable dosage unit formulations containing conventional non toxic pharmaceutically acceptable diluents, excipients or carriers appropriate for each route of administration. When the particular second therapeutic agent and the insulinotropic peptide conjugate are administered separately, they can be administered by different routes.

The formulation can be administered at any injection site deemed suitable by the practitioner of skill. In certain embodiments, the formulation is administered in the abdomen, thigh or arm.

The formulation can be administered at any time deemed suitable by the practitioner of skill. In certain embodiments, the formulation is administered in the morning, before a meal or in the evening prior to sleep, or a combination thereof.

It will be understood, however, that the specific dose level and frequency of dosage for any particular subject can be varied and will depend upon a variety of factors including the age, body weight, general health, sex, diet, mode and time of administration, rate of excretion, drug combination, the severity of the particular condition, and the host undergoing therapy.

In the event that the subject should experience adverse events in response to one or more agents of the combination therapy provided herein, for example, nausea, vomiting, injection-related skin reaction, hypoglycemia, i.e., blood glucose level, 60 mg/dL (3.3 mmol/L) with clinical signs of hypoglycemia, or any other constitutional symptoms or signs, such as extreme and rapid weight loss, the specific dose level and frequency of dosage for one or more of the agents can be reduced or adjusted according to the judgment of the practitioner of skill in the art.

In a particular embodiment of the combination therapy provided herein, the subject receives the insulinotropic peptide conjugate and an OAD, e.g., a biguanide, e.g., metformin. In another particular embodiment, the subject receives the insulinotropic peptide conjugate, and two OADs, e.g., a biguanide, e.g., metformin, sulfonylurea or a thiazolidinedione, and a second OAD.

### 5.4 Kits

In a further embodiment, the present invention provides kits comprising an insulinotropic peptide conjugate, e.g., insulinotropic peptide conjugate formulation, of the invention, which can be used, for instance, in practicing the methods of treatment described herein. For example, the present invention provides kits for the treatment of type II diabetes mellitus in a subject in need thereof. The kits comprise an insulinotropic peptide conjugate, e.g., insulinotropic peptide conjugate formulation, in a package for distribution to a practitioner of skill in the art. The kits can comprise a label or labeling with instructions for use of the insulinotropic conjugate as described herein, e.g, instructions for administering the insulinotropic peptide conjugate, e.g., insulinotropic peptide conjugate formulation, for the treatment of subjects with (or who are or are undergoing), e.g. pre-diabetes (e.g., impaired glucose tolerance (IGT) and impaired fasting glucose (IFG)), diabetes, e.g., type I diabetes or type II diabetes, late autoimmune diabetes in adults (“LADA”) also known as late onset autoimmune diabetes of adulthood, slow onset type I diabetes and type 1.5 diabetes, steroid induced diabetes, Human Immunodeficiency Virus (HIV) Treatment-Induced Diabetes, diabetes development in subjects with congenital or HIV-Associated Lipodystrophy (“Fat Redistribution Syndrome”), obesity (i.e., BMI of 30 kg/m² or greater), overweight (i.e., BMI between 25 kg/m² and 30 kg/m²), metabolic syndrome (Syndrome X), nervous system disorders, surgery, insulin resistance, hypoglycemia unawareness, restrictive lung disease, gastrointestinal disorders, e.g., irritable bowel syndrome (IBS), functional dyspepsia, pain associated with gastrointestinal disorders, e.g., pain associated with IBS and functional dyspepsia, inflammatory bowel disease (IBD), e.g., Crohn's disease and ulcerative colitis, pain associated with IBD, hyperglycemia, e.g., hyperglycemia associated with surgery (e.g., a major surgical procedure, e.g., coronary bypass surgery) e.g., hyperglycemia associated with surgery on subjects with diabetes, e.g., type II diabetes, metabolic syndrome, coronary heart failure (CHF), disorders associated with beta cell disfunction, disorders associated with the absence of beta cells, disorders associated with insufficient numbers of beta cells, and other conditions treatable with an insulinotropic peptide or insulinotropic peptide conjugate.

The kits can comprise a label or labeling with instructions for use of the insulinotropic conjugate as described herein, e.g, instructions for administering the insulinotropic peptide conjugate, e.g., insulinotropic peptide conjugate formulation, to promote weight loss, stimulate insulin synthesis and release, to enhance adipose, muscle or liver tissue sensitivity toward insulin uptake, to stimulate glucose uptake, to slow (e.g., decrease the rate of) digestive processes, e.g., gastric emptying, to block or inhibit secretion of glucagon, to promote beta cell function, proliferation, and/or activity, to restore first phase insulin release in subjects with diabetes, to reduce food intake, to reduce appetite, to prevent or protect against liver disease, e.g., liver disease associated with obesity, diabetes, or hyperglycemia (e.g., non-alcoholic fatty liver disease (NAFLD), non-alcoholic steatohepatitis (NASH)).

The instructions on the label can further include instructions for storage conditions of the insulinotropic peptide conjugates as described herein.

In certain embodiments, the kit can comprise one or more containers, e.g., bottles, vials, ampoules, pre-filled containers, e.g., pre-filled syringes or prefilled injection pens, microchip (e.g., a microchip for controlled release of its contents) or test tubes which contain a unit dosage or a multi-use dosage of the insulinotropic peptide conjugate, e.g., insulotropic peptide conjugate formulation. The dosage forms can be contained as liquid or lyophilized formulations. Kits comprising lyophilized dosage forms can further comprise one or more additional containers comprising a diluent for reconstituting the lyophilized formulation, such that the protein, insulinotropic peptide conjugate, concentration in the reconstituted formulation is at least 1, 2, 3, 4, 5, 10, 20, 30, 40, 50 mg/ml, for example from about 1 mg/ml to about 100 mg/ml, more preferably from about 1 mg/ml to about 50 mg/ml, and most preferably from about 1 mg/ml to about 15 mg/ml.

The kit can further comprise one or more additional components useful for carrying out the methods of treatment described herein, including, but not limited to, buffers, filters, needles, syringes, and package inserts with instructions for use. In a particular embodiment, the kit comprises a needle, e.g., a 25-gauge needle, a 26-gauge needle, a 27-gauge needle, a 28-gauge needle, a 29-gauge needle, a 30-gauge needle, a 31-gauge needle, a 32-gauge needle, or a 33-gauge needle, or a higher gauge needle, useful, e.g. for the subcutaneous administration of the insulinotropic peptide conjugate formulation to a subject. In certain embodiments, the kits can comprise components useful for the safe disposal of means for administering the insulinotropic peptide conjugate formulation, e.g. a sharps container for used syringes and needles.

In a preferred embodiment, the kit comprises one or more syringes pre-loaded with a first dosage of the insulinotropic peptide conjugate, e.g., insulinotropic peptide conjugate formulation, and one or more syringes pre-loaded with a second higher dosage, of the insulinotropic peptide conjugate, e.g., insulinotropic peptide conjugate formulation, useful e.g., for administering increasing dosages to a subject during the course of a dosing regimen described herein. In a particular embodiment, the kit comprises 1, 2, 3, 4, 5, 6, 7, 8, or more than 8 syringes pre-loaded with a first dosage of the insulinotropic peptide conjugate, e.g., insulinotropic peptide conjugate formulation. In another particular embodiment, the kit comprises 1, 2, 3, 4, 5, 6, 7, 8, or more than 8 syringes pre-loaded with a second higher dosage of the insulinotropic peptide conjugate, e.g., insulinotropic peptide conjugate formulation.

In some embodiments, syringes pre-loaded with a first dosage comprise the insulinotropic peptide conjugate in an amount of about 1000 μg. In some embodiments, syringes pre-loaded with a first dosage comprise the insulinotropic peptide conjugate in an amount of about 1500 μg. In some embodiments, syringes pre-loaded with a second higher dosage comprise the insulinotropic peptide conjugate in an amount of about 2000 μg.

In other embodiments, the kit comprises one, two, three, four, five, six, seven, eight, nine, ten or more than ten empty syringes, and one, two, three, four, five, six, seven, eight, nine, ten or more than ten vials, wherein each vial contains 1 dose, 2 doses, 3 doses, 4 doses, 5 doses, 6 doses, 7 doses, 8 doses, 9 doses, 10 doses or more than 10 doses of the insulinotropic peptide conjugate formulation. In other embodiments, the kit comprises one, two, three, four, five, six, seven, eight, nine, ten or more than ten syringes pre-loaded with 1 dose, 2 doses, 3 doses, 4 doses, 5 doses, 6 doses, 7 doses, 8 doses, 9 doses, 10 doses, or more than 10 doses of the insulinotropic peptide conjugate formulation. In some embodiments, the syringe comprises a luer-lock, luer-cone, or other needle fitting connector that facilitates attachment of a disposable needle. In other embodiments, the syringe comprises a staked, i.e., permanent, needle.

In a particular embodiment, the kit comprises a pen-type delivery apparatus and one, two, three, four, five, six, seven, eight, nine, ten or more than ten replaceable cartridges, wherein the replaceable cartridge comprises, e.g., is pre-loaded with 1 dose, 2 doses, 3 doses, 4 doses, 5 doses, 6 doses, 7 doses, 8 doses, 9 doses, 10 doses or more than 10 doses of the insulinotropic peptide conjugate formulation. In certain embodiments where the pen-type delivery apparatus comprises multiple doses, the dose can be pre-set, i.e., fixed. In other embodiments, the dose can be a flexible dose, i.e., dialed-in by the user. In a particular embodiment, the kit comprises one, two, three, four, five, six, seven, eight, nine, ten or more than ten pen-type delivery apparatuses pre-loaded with one, two, three, four, five, six, seven, eight, nine, ten or more than ten doses of the insulinotropic peptide conjugate formulation. In some embodiments, the pen-type delivery apparatus comprises a luer-lock, luer-cone, or other needle fitting connector that facilitates attachment of a disposable needle. In a particular embodiment, the kit comprises a disposable pen-type delivery apparatus. In other embodiments, the pen-type delivery apparatus comprises a staked, i.e., permanent, needle. In some embodiments, the insulinotropic peptide conjugate formulation comprises 10 mg/ml exendin-4(1-39) Lys⁴⁰ (ε-AEEA-MPA)-NH₂ albumin conjugate in 10 mM sodium acetate buffer at pH 5.0, containing 5 mM sodium octanoate, 0.1% (w/v) pluronic F68 and 150 mM sodium chloride. In other embodiments, the insulinotropic peptide conjugate formulation comprises 10 mg/ml exendin-4(1-39) Lys⁴⁰ (ε-AEEA-MPA)-NH₂ albumin conjugate in 10 mM sodium phosphate buffer at pH 7.0, containing 1.6 mM sodium octanoate, 15 mg/L polysorbate 80, and 135 mM sodium chloride.

### 5.5 Insulinotropic Peptide Conjugates

The invention is directed to pharmaceutical formulations comprising an insulinotropic peptide conjugate. Useful insulinotropic peptides include, but are not limited to, GLP-1, exendin-3 and exendin-4, and their precursors, derivatives and fragments. Such insulinotropic peptides include those disclosed in U.S. Pat. Nos. 6,514,500; 6,821,949; 6,887,849; 6,849,714; 6,329,336; 6,924,264; WO 03/103572 and 6,593,295, the contents of each of which are incorporated by reference herein in their entireties.

In a preferred embodiment, the insulinotropic peptide is a C-terminal amide (CO—NH₂).

In some embodiments, the insulinotropic peptide is GLP-1. In some embodiments, the insulinotropic peptide is a GLP-1 derivative. In some embodiments, the insulinotropic peptide is exendin-3. In some embodiments, the insulinotropic peptide is an exendin-3 derivative. In some embodiments, the insulinotropic peptide is exendin-4. In some embodiments, the insulinotropic peptide is an exendin-4 derivative. In some embodiments, the insulinotropic peptide is exendin-4(1-39)-NH₂. In some embodiments, the insulinotropic peptide is exendin-4(1-39)Lys⁴⁰-NH₂.

In a preferred embodiment, the insulinotropic peptide conjugate is exendin-4(1-39) Lys⁴⁰ (ε-AEEA-MPA)-NH₂ albumin conjugate.

5.5.1 GLP-1 and Its Derivatives

The hormone glucagon can be synthesized according to any method known to those of skill in the art. In some embodiments, it is synthesized as a high molecular weight precursor molecule which is subsequently proteolytically cleaved into three peptides: glucagon, GLP-1, and glucagon-like peptide 2 (GLP-2). GLP-1 has 37 amino acids in its unprocessed form as shown in SEQ ID NO: 1 (HDEFERHAEG TFTSDVSSYL EGQAAKEFIA WLVKGRG). Unprocessed GLP-1 is essentially unable to mediate the induction of insulin biosynthesis. The unprocessed GLP-1 peptide is, however, naturally converted to a 31-amino acid long peptide (7-37 peptide) having amino acids 7-37 of GLP-1 (“GLP-1(7-37)”) SEQ ID NO:2 (HAEG TFTSDVSSYL EGQAAKEFIA WLVKGRG). GLP-1(7-37) can also undergo additional processing by proteolytic removal of the C-terminal glycine to produce GLP-1(7-36) which also exists predominantly with the C-terminal residue, arginine, in amidated form as arginineamide, GLP-1(7-36) amide. This processing occurs in the intestine and to a much lesser extent in the pancreas, and results in a polypeptide with the insulinotropic activity of GLP-1(7-37).

A compound is said to have an “insulinotropic activity” if it is able to stimulate, or cause the stimulation of, the synthesis or expression of the hormone insulin. The hormonal activity of GLP-1(7-37) and GLP-1(7-36) appear to be specific for the pancreatic beta cells where it appears to induce the biosynthesis of insulin. Glucagon-like-peptide hormones are useful in the study of the pathogenesis of maturity onset diabetes mellitus, a condition characterized by hyperglycemia in which the dynamics of insulin secretion are abnormal. Moreover, glucagon-like peptides are useful in the therapy and treatment of this disease, and in the therapy and treatment of hyperglycemia.

Peptide moieties (fragments) can be chosen from the determined amino acid sequence of human GLP-1. The interchangeable terms “peptide fragment” and “peptide moiety” are meant to include both synthetic and naturally occurring amino acid sequences derivable from a naturally occurring amino acid sequence.

The amino acid sequence for GLP-1 has been reported by several researchers. See Lopez, L. C. et al., 1983, Proc. Natl. Acad. Sci., USA 80:5485-5489; Bell, G. I. et al., 1983, Nature 302:716-718; Heinrich, G. et al., 1984, Endocrinol. 115:2176-2181. The structure of the preproglucagon mRNA and its corresponding amino acid sequence is well known. The proteolytic processing of the precursor gene product, proglucagon, into glucagon and the two insulinotropic peptides has been characterized. As used herein, the notation of GLP-1(1-37) refers to a GLP-1 polypeptide having all amino acids from 1 (N-terminus) through 37 (C-terminus). Similarly, GLP-1(7-37) refers to a GLP-1 polypeptide having all amino acids from 7 (N-terminus) through 37 (C-terminus). Similarly, GLP-1(7-36) refers to a GLP-1 polypeptide having all amino acids from number 7 (N-terminus) through number 36 (C-terminus).

In one embodiment, GLP-1(7-36) and its peptide fragments are synthesized by conventional means as detailed below, such as by the well-known solid-phase peptide synthesis described by Merrifield, J. M., 1962, Chem. Soc. 85:2149, and Stewart and Young, Solid Phase Peptide Synthesis, Freeman, San Francisco, 1969, pp. 27-66), the contents of each of which are incorporated by reference herein in their entireties. However, it is also possible to obtain fragments of the proglucagon polypeptide, or of GLP-1, by fragmenting the naturally occurring amino acid sequence, using, for example, a proteolytic enzyme. Further, it is possible to obtain the desired fragments of the proglucagon peptide or of GLP-1 through the use of recombinant DNA technology, as disclosed by Maniatis, T., et al., Molecular Biology: A Laboratory Manual, Cold Spring Harbor, N.Y., 1982, which is hereby incorporated by reference herein in its entirety.

Useful peptides for the methods described herein include those which are derivable from GLP-1 such as GLP-1(1-37) and GLP-1(7-36). A peptide is said to be “derivable from a naturally occurring amino acid sequence” if it can be obtained by fragmenting a naturally occurring sequence, or if it can be synthesized based upon a knowledge of the sequence of the naturally occurring amino acid sequence or of the genetic material (DNA or RNA) which encodes this sequence.

Also useful are those molecules which are said to be “derivatives” of GLP-1 such as GLP-1(1-37) and especially GLP-1(7-36). Such a “derivative” has the following characteristics: (1) it shares substantial homology with GLP-1 or a similarly sized fragment of GLP-1; (2) it is capable of functioning as an insulinotropic hormone; and (3) using at least one of the assays provided herein, the derivative has an insulinotropic activity of at least 1%, 5%, 10%, 25% 50%, 75%, 100%, or greater than 100% of the insulinotropic activity of GLP-1.

A derivative of GLP-1 is said to share “substantial homology” with GLP-1 if the amino acid sequences of the derivative shares at least 80%, and more preferably at least 90%, and most preferably at least 95% identity to GLP-1(1-37). Percent identity in this context means the percentage of amino acid residues in the candidate sequence that are identical (i.e., the amino acid residues at a given position in the alignment are the same residue) or similar (i.e., the amino acid substitution at a given position in the alignment is a conservative substitution, as discussed above), to the corresponding amino acid residue in the peptide after aligning the sequences and introducing gaps, if necessary, to achieve the maximum percent sequence homology. In certain embodiments, a GLP-1 derivative is characterized by its percent sequence identity or percent sequence similarity with the naturally occurring GLP-1 sequence. Sequence homology, including percentages of sequence identity and similarity, are determined using sequence alignment techniques well-known in the art, preferably computer algorithms designed for this purpose, using the default parameters of said computer algorithms or the software packages containing them.

Useful derivatives also include GLP-1 fragments which, in addition to containing a sequence that is substantially homologous to that of a naturally occurring GLP-1 peptide may contain one or more additional amino acids at their amino and/or their carboxy termini, or internally within said sequence. Thus, useful derivatives include polypeptide fragments of GLP-1 that may contain one or more amino acids that may not be present in a naturally occurring GLP-1 sequence provided that such polypeptides have an insulinotropic activity of at least 1%, 5%, 10%, 25% 50%, 75%, 100%, or greater than 100% of the insulinotropic activity of GLP-1. The additional amino acids may be D-amino acids or L-amino acids or combinations thereof.

Useful GLP-1 fragments also include those which, although containing a sequence that is substantially homologous to that of a naturally occurring GLP-1 peptide, lack one or more additional amino acids at their amino and/or their carboxy termini that are naturally found on a GLP-1 peptide. Thus, useful polypeptide fragments of GLP-1 may lack one or more amino acids that are normally present in a naturally occurring GLP-1 sequence provided that such polypeptides have an insulinotropic activity of at least 1%, 5%, 10%, 25% 50%, 75%, 100%, or greater than 100% of the insulinotropic activity of GLP-1. In certain embodiments, the polypeptide fragments lack one amino acid normally present in a naturally occurring GLP-1 sequence. In some embodiments, the polypeptide fragments lack two amino acids normally present in a naturally occurring GLP-1 sequence. In some embodiments, the polypeptide fragments lack three amino acids normally present in a naturally occurring GLP-1 sequence. In some embodiments, the polypeptide fragments lack four amino acids normally present in a naturally occurring GLP-1 sequence.

Also useful are obvious or trivial variants of the above-described fragments which have inconsequential amino acid substitutions (and thus have amino acid sequences which differ from that of the natural sequence) provided that such variants have an insulinotropic activity which is substantially identical to that of the above-described GLP-1 derivatives. Examples of obvious or trivial substitutions include the substitution of one basic residue for another (i.e. Arg for Lys), the substitution of one hydrophobic residue for another (i.e. Leu for Ile), or the substitution of one aromatic residue for another (i.e. Phe for Tyr), etc.

In addition to those GLP-1 derivatives with insulinotropic activity, GLP-1 derivatives which stimulate glucose uptake by cells but do not stimulate insulin expression or secretion are useful for the methods described herein. Such GLP-1 derivatives are described in U.S. Pat. No. 5,574,008, which is hereby incorporated by reference herein in its entirety.

GLP-1 derivatives which stimulate glucose uptake by cells but do not stimulate insulin expression or secretion which find use in the methods described herein

(SEQ ID NO:3)

H₂N-Ser-Tyr-Leu-Glu-Gly-Gln-Ala-Ala-Lys-Glu-Phe-
Ile-Ala-Trp-Leu-Val-Xaa-Gly-Arg-R²;

(SEQ ID NO:4)

H₂N-Ser-Ser-Tyr-Leu-Glu-Gly-Gln-Ala-Ala-Lys-Glu-
Phe-Ile-Ala-Trp-Leu-Val-Xaa-Gly-Arg-R²;

(SEQ ID NO:5)

H₂N-Val-Ser-Ser-Tyr-Leu-Glu-Gly-Gln-Ala-Ala-Lys-
Glu-Phe-Ile-Ala-Trp-Leu-Val-Xaa-Gly-Arg-R²;

(SEQ ID NO:6)

H₂N-Asp-Val-Ser-Ser-Tyr-Leu-Glu-Gly-Gln-Ala-Ala-
Lys-Glu-Phe-Ile-Ala-Trp-Leu-Val-Xaa-Gly-Arg-R²;

(SEQ ID NO:7)

H₂N-Ser-Asp-Val-Ser-Ser-Tyr-Leu-Glu-Gly-Gln-Ala-
Ala-Lys-Glu-Phe-Ile-Ala-Trp-Leu-Val-Xaa-Gly-Arg-
R²;

(SEQ ID NO:8)

H₂N-Thr-Ser-Asp-Val-Ser-Ser-Tyr-Leu-Glu-Gly-Gln-
Ala-Ala-Lys-Glu-Phe-Ile-Ala-Trp-Leu-Val-Xaa-Gly-
Arg-R²;

(SEQ ID NO:9)

H₂N-Phe-Thr-Ser-Asp-Val-Ser-Ser-Tyr-Leu-Glu-Gly-
Gln-Ala-Ala-Lys-Glu-Phe-Ile-Ala-Trp-Leu-Val-Xaa-
Gly-Arg-R²;

(SEQ ID NO:10)

H₂N-Thr-Phe-Thr-Ser-Asp-Val-Ser-Ser-Tyr-Leu-Glu-
Gly-Gln-Ala-Ala-Lys-Glu-Phe-Ile-Ala-Trp-Leu-Val-
Xaa-Gly-Arg-R²;

(SEQ ID NO:11)

H₂N-Gly-Thr-Phe-Thr-Ser-Asp-Val-Ser-Ser-Tyr-Leu-
Glu-Gly-Gln-Ala-Ala-Lys-Glu-Phe-Ile-Ala-Trp-Leu-
Val-Xaa-Gly-Arg-R²;

(SEQ ID NO:12)

H₂N-Glu-Gly-Thr-Phe-Thr-Ser-Asp-Val-Ser-Ser-Tyr-
Leu-Glu-Gly-Gln-Ala-Ala-Lys-Glu-Phe-Ile-Ala-Trp-
Leu-Val-Xaa-Gly-Arg-R²;

(SEQ ID NO:13)

H₂N-Ala-Glu-Gly-Thr-Phe-Thr-Ser-Asp-Val-Ser-Ser-
Tyr-Leu-Glu-Gly-Gln-Ala-Ala-Lys-Glu-Phe-IIe-Ala-
Trp-Leu-Val-Xaa-Gly-Arg-R²;

(SEQ ID NO:14)

H₂N-His-Ala-Glu-Gly-Thr-Phe-Thr-Ser-Asp-Val-Ser-
Ser-Tyr-Leu-Glu-Gly-Gln-Ala-Ala-Lys-Glu-Phe-Ile-
Ala-Trp-Leu-Val-Xaa-Gly-Arg-R²;
and

(SEQ ID NO:15)

H₂N-His-D-Ala-Glu-Gly-Thr-Phe-Thr-Ser-Asp-Val-Ser-
Ser-Tyr-Leu-Glu-Gly-Gln-Ala-Ala-Lys-Glu-Phe-Ile-
Ala-Trp-Leu-Val-Xaa-Gly-Arg-R².

These peptides are C-terminal GLP-1 fragments which do not have insulinotropic activity but which are nonetheless useful for treating diabetes and hyperglycemic conditions as described in U.S. Pat. No. 5,574,008, which is hereby incorporated by reference herein in its entirety.

An additional GLP-1 derivative which finds use in the formulations and methods described herein includes a GLP-1/exendin-4 hybrid peptide comprising GLP-1(7-36) fused to the nine C-terminal amino acids of exendin-4, having the sequence:

(SEQ ID NO:28)

HAEG TFTSDVSSYL EGQAAKEFIA WLVKGRPSSGAPPPS.

Also useful in the formulations and methods described herein is the GLP-1 derivative comprising a fusion protein molecule as follows: [Gly⁸]GLP-1(7-36)-[Gly⁸]GLP-1(7-36)-human serum albumin (albiglutide), as described in U.S. Pat. No. 7,141,547, which is hereby incorporate by reference in its entirety.

Additional GLP-1 derivatives which find use in the formulations and methods described herein include the following GLP-1 fusion protein molecules: GLP-1(7-36)-human serum albumin; human serum albumin-GLP-1(7-36); [Gly⁸]GLP-1(7-36)-human serum albumin; human serum albumin-[Gly⁸]GLP-1(7-36); GLP-1(7-36)-GLP-1(7-36)-human serum albumin; GLP-1(9-36)-human serum albumin; and [Gly⁸]GLP-1(7-36)-GLP-1(7-36)-human serum albumin, as described in U.S. Pat. No. 7,141,547, which is hereby incorporated by reference herein in its entirety.

An additional GLP-1 derivative which finds use in the formulations and methods described herein includes a GLP-1/exendin-4/human serum albumin hybrid polypeptide, comprising [Gly⁸][Glu²²]GLP-1(7-36) fused to the eight C-terminal amino acids of exendin-4(1-39), fused to a linker sequence, fused to human serum albumin, having the sequence: HGEGTFTSDV SSYLEEQAAK EFIAWLVKGR GSSGAPPPSG GGGGSGGGGS GGGGSDAHKS EVAHRFKDLG EENFKALVLI AFAQYLQQCP FEDHVKLVNE VTEFAKTCVA DESAENCDKS LHTLFGDKLC TVATLRETYG EMADCCAKQE PERNECFLQH KDDNPNLPRL VRPEVDVMCT AFHDNEETFL KKYLYEIARR HPYFYAPELL FFAKRYKAAF TECCQAADKA ACLLPKLDEL RDEGKASSAK QRLKCASLQK FGERAFKAWA VARLSQRFPK AEEAEVSKLV TDLTKVHTEC CHGDLLECAD DRADLAKYIC ENQDSISSKL KECCEKPLLE KSHClAEVEN DEMPADLPSL AADFVESKDV CKNYAEAKDV FLGMFLYEYA RRHPDYSVVL LLRLAKTYET TLEKCCAAAD PHECYAKVFD EFKPLVEEPQ NLIKQNCELF EQLGEYKFQN ALLVRYTKKV PQVSTPTLVE VSRNLGKVGS KCCKHPEAKR MPCAEDYLSV VLNQLCVLHE KTPVSDRVTK CCTESLVNRR PCFSALEVDE TYVPKEFNAE TFTFHADICT LSEKERQIKK QTALVELVKH KPKATKEQLK AVMDDFAAFV EKCCKADDKE TCFAEEGKKL VAASQAALGL (SEQ ID NO:29), as described in U.S. Pat. No. 7,271,149, which is hereby incorporate by reference in its entirety.

5.5.2 Exendin-3 and Exendin-4 Peptides and Derivatives

Exendin-3 and exendin-4 are 39 amino acid peptides (differing at residues 2 and 3) which are approximately 53% homologous to GLP-1 and find use as insulinotropic agents.

The amino acid sequence of exendin-3 is HSDGTFTSDLSKQMEEEAVRLFIEWLKNGGPSSGAPPPS (SEQ ID NO: 16), and the amino acid sequence of exendin-4 is HGEGTFTSDLSKQMEEEAVRLFIEWLKNGGPSSGAPPPS (SEQ ID NO: 17).

Also useful for the formulations described herein are insulinotropic fragments of exendin-4 comprising the amino acid sequences: exendin-4(1-31) desGlu¹⁷ Tyr³² (SEQ ID NO: 18) HGEGTFTSDLSKQMEEAVRLFIEWLKNGGPY and exendin-4(1-30) Tyr³¹ (SEQ ID NO: 19) HGEGTFTSDLSKQMEEEAVRLFIEWLKNGGY.

Also useful is the inhibitory fragment of native exendin-4 comprising the amino acid sequence: exendin-4(9-39) (SEQ ID NO:20) DLSKQMEEEAVRLFIEWLKNGGPSSGAPPPS.

Other exemplary insulinotropic peptides are presented in SEQ ID NOS:21-27.

SEQ ID NO:21

HDEFERHAEGTFTSDVSSYLEGQAAKEFIAWLVKGRK

SEQ ID NO:22

HAEGTFTSDVSSYLEGQAAKEFIAWLVKGRK

SEQ ID NO:23

HGEGTFTSDLSKQMEEEAVRLFIEWLKNGGPSSGAPPPSK

SEQ ID NO:24

HSDGTFTSDLSKQMEEEAVRLFIEWLKNGGPSSGAPPPSK

SEQ ID NO:25

HGEGTFTSDLSKEMEEEVRLFIEWLKNGGPY

SEQ ID NO:26

HGEGTFTSDLSKEMEEEVRLFIEWLKNGGY

SEQ ID NO:27

DLSKQMEEEAVRLFIEWLKGGPSSGPPPS

Useful peptides for the formulations described herein include peptides which are derivable from the naturally occurring exendin-3 and exendin-4 peptides. A peptide is said to be “derivable from a naturally occurring amino acid sequence” if it can be obtained by fragmenting a naturally occurring sequence, or if it can be synthesized based upon a knowledge of the sequence of the naturally occurring amino acid sequence or of the genetic material (DNA or RNA) which encodes this sequence.

Useful molecules for the formulations described herein also include those which are said to be “derivatives” of exendin-3 and exendin-4. In one embodiment of the invention, a “derivative” has the following characteristics: (1) it shares substantial homology with exendin-3 or exendin-4 or a similarly sized fragment of exendin-3 or exendin-4; (2) it is capable of functioning as an insulinotropic hormone and (3) using at least one of the assays provided herein, the derivative has an insulinotropic activity of at least 1%, 5%, 10%, 25% 50%, 75%, 100%, or greater than 100% of the insulinotropic activity of either exendin-3 or exendin-4.

A derivative of exendin-3 or exendin-4 is said to share “substantial homology” with exendin-3 and exendin-4 if the amino acid sequences of the derivative shares at least 80%, and more preferably at least 90%, and most preferably at least 95% identity to exendin-3 and exendin-4. Percent identity in this context means the percentage of amino acid residues in the candidate sequence that are identical (i.e., the amino acid residues at a given position in the alignment are the same residue) or similar (i.e., the amino acid substitution at a given position in the alignment is a conservative substitution, as discussed above), to the corresponding amino acid residue in the native peptide after aligning the sequences and introducing gaps, if necessary, to achieve the maximum percent sequence homology. In certain embodiments, a exendin-3 or exendin-4 derivative is characterized by its percent sequence identity or percent sequence similarity with the naturally occurring exendin-3 or exendin-4 sequence. Sequence homology, including percentages of sequence identity and similarity, are determined using sequence alignment techniques well-known in the art, preferably computer algorithms designed for this purpose, using the default parameters of said computer algorithms or the software packages containing them.

Useful derivatives also include exendin-3 or exendin-4 fragments which, in addition to containing a sequence that is the same or that is substantially homologous to that of a naturally occurring exendin-3 or exendin-4 peptide may contain one or more additional amino acids at their amino and/or their carboxy termini, or internally within said sequence. Thus, useful derivatives include polypeptide fragments of exendin-3 or exendin-4 that may contain one or more amino acids that may not be present in a naturally occurring exendin-3 or exendin-4 sequences provided that such polypeptides have an insulinotropic activity of at least 1%, 5%, 10%, 25% 50%, 75%, 100%, or greater than 100% of the insulinotropic activity of either exendin-3 or exendin-4.

Similarly, useful derivatives include exendin-3 or exendin-4 fragments which, although containing a sequence that is substantially homologous to that of a naturally occurring exendin-3 or exendin-4 peptide may lack one or more additional amino acids at their amino and/or their carboxy termini that are naturally found on a exendin-3 or exendin-4 peptide. Thus, useful derivatives include polypeptide fragments of exendin-3 or exendin-4 that may lack one or more amino acids that are normally present in a naturally occurring exendin-3 or exendin-4 sequence provided that such polypeptides have an insulinotropic activity of at least 1%, 5%, 10%, 25% 50%, 75%, 100%, or greater than 100% of the insulinotropic activity of either exendin-3 or exendin-4.

Useful derivatives further include exendin-3 or exendin-4 fragments which are otherwise identical in sequence to that of the naturally occurring exendin-3 or exendin-4 peptide but for the addition, deletion or substitution of no more than 5, 4, 3, 2 or 1 amino acids. In certain embodiments, the derivative contains no more than 5, no more than 4, no more than 3, no more than 2, or no more than 1 amino addition, deletion, or substitution relative to the native exendin-3 or exendin-4 sequence. Thus, useful derivatives include polypeptide fragments of exendin-3 or exendin-4 that are identical but for no more than 5, 4, 3, 2, or 1 amino acid additions, deletions or substitutions relative to the native exendin-3 or exendin-4 sequence, provided that such polypeptides have an insulinotropic activity of at least 1%, 5%, 10%, 25% 50%, 75%, 100%, or greater than 100% of the insulinotropic activity of either exendin-3 or exendin-4.

Useful derivatives also include conservative variants of the above-described fragments which have inconsequential amino acid substitutions (and thus have amino acid sequences which differ from that of the natural sequence) provided that such variants still have an insulinotropic activity. Examples of conservative substitutions include the substitution of one basic residue for another (i.e. Arg for Lys), the substitution of one hydrophobic residue for another (i.e. Leu for Ile), or the substitution of one aromatic residue for another (i.e. Phe for Tyr), etc. The following six groups each contain amino acids that are conservative substitutions for one another:
Alanine (A), Serine (S), and Threonine (T)
Aspartic acid (D) and Glutamic acid (E)
Asparagine (N) and Glutamine (Q)
Arginine (R) and Lysine (K)
Isoleucine (I), Leucine (L), Methionine (M), and Valine (V)
Phenylalanine (F), Tyrosine (Y), and Tryptophan (W).

Also useful in the formulations and methods described herein are the exendin-4 derivatives comprising a fusion protein molecule as follows: exendin-4(1-39)-human serum albumin, and human serum albumin-exendin-4(1-39), as described in U.S. Pat. No. 7,141,547 or 7,271,149, the contents of each of which are incorporated by reference herein in their entireties.

5.5.3 Conjugates of Insulinotropic Peptides to Albumin

Useful insulinotropic peptide conjugates of the pharmaceutical formulation described herein include insulinotropic peptides and their derivatives conjugated to albumin. Several methods can be used to link an insulinotropic peptide to albumin. In certain embodiments, the insulinotropic peptide is linked to albumin according to any technique known to those of skill in the art. In some embodiments, the insulinotropic peptide is modified to include a reactive group which can react with available reactive functionalities on albumin to form covalent linkages.

The reactive group is chosen for its ability to form a stable covalent bond with albumin, for example, by reacting with one or more amino groups, hydroxyl groups, or thiol groups on the serum protein or peptide. Preferably, a reactive group reacts with only one amino group, hydroxyl group, or thiol group on albumin. Preferably, a reactive group reacts with a specific amino group, hydroxyl group, or thiol group on albumin. A useful conjugate of the methods described herein comprises a modified peptide, or a modified derivative thereof, which is covalently attached to albumin via a reaction of the reactive group with an amino group, hydroxyl group, or thiol group on albumin. Thus, a useful conjugate comprises a modified peptide, or a modified derivative thereof, in which the reactive group has formed a covalent bond to albumin.

To form covalent bonds with the functional group on a protein, one may use as a chemically reactive group a wide variety of active carboxyl groups, particularly esters. While a number of different hydroxyl groups may be employed in these linking agents, the most convenient would be N-hydroxysuccinimide (NHS), N-hydroxy-sulfosuccinimide (sulfo-NHS), maleimide-benzoyl-succinimide (MBS), gamma-maleimido-butyryloxy succinimide ester (GMBS) and 3-maleimidopropionic acid (3-MPA).

Primary amines are the principal targets for NHS esters. Accessible α-amine groups present on the N-termini of proteins react with NHS esters. However, ε-amino groups on a protein may not be desirable or available for the NHS coupling. While five amino acids have nitrogen in their side chains, only the ε-amine of lysine reacts significantly with NHS esters. An amide bond can form when the NHS ester conjugation reaction reacts with primary amines releasing N-hydroxysuccinimide. These succinimide containing reactive groups are herein referred to as succinimidyl groups.

In particular embodiments, the functional group on albumin is the single free thiol group located at amino acid residue 34 (Cys34) and the chemically reactive group is a maleimido-containing group such as (GMBA or MPA). GMBA stands for gamma-maleimide-butrylamide. Such maleimide containing groups are referred to herein as maleimido groups.

In some embodiments, albumin is covalently linked to a succinimidyl or maleimido group on the insulinotropic peptide. In some embodiments, an albumin amino, hydroxyl or thiol group is covalently linked to a succinimidyl or maleimido group on the insulinitropic peptide. In some embodiments, albumin cysteine 34 thiol is covalently linked to a [2-[2-[2-maleimidopropionamido(ethoxy)ethoxy]acetic acid linker on the epsilon amino of a lysine of the insulinotropic peptide.

In a specific embodiment, the reactive group is a single MPA reactive group attached to the peptide, optionally through a linking group, at a single defined amino acid and the MPA is covalently attached to albumin at substantially a single amino acid residue of albumin, preferably cysteine 34. In a preferred embodiment, the albumin is recombinant human albumin. In certain embodiments, the reactive group, preferably MPA, is attached to the peptide through one or more linking groups, preferably AEEA, AEA, or amino-octanoic acid, more particularly 8-amino-octanoic acid. In certain examples of embodiments in which the reactive group, preferably MPA, is attached to the peptide through more than one linking group, each linking group can be independently selected from the group consisting preferably of AEA ((2-amino) ethoxy acetic acid), AEEA ([2-(2-amino)ethoxy)]ethoxy acetic acid), and amino-octanoic acid, more particularly 8-amino-octanoic acid. In one embodiment, the reactive group, preferably MPA, is attached to the peptide via 1, 2, 3, 4, 5 or 6 AEEA linking groups which are arranged in tandem. In another embodiment, the reactive group, preferably MPA, is attached to the peptide via 1, 2, 3, 4, 5 or 6 8-amino-octanoic acid linking groups which are arranged in tandem.

In certain embodiments, the reactive group can be attached to any residue of the insulinotropic peptide suitable for attachment of such a reactive group. The residue can be a terminal or internal residue of the peptide. In certain embodiments, the reactive group can be attached to the carboxy-terminus or amino-terminus of the peptide. In advantageous embodiments, the reactive group is attached to a single site of the peptide. This can be achieved using protecting groups known to those of skill in the art. In certain embodiments, a derivative of the insulinotropic peptide can comprise a residue incorporated for attachment of the reactive group. Useful residues for attachment include, but are not limited to, lysine, aspartate and glutamate residues. The residue can be incorporated internally or at a terminus of the peptide. In certain embodiments, the reactive group is attached to an internal lysine residue. In certain embodiments, the reactive group is attached to a terminal lysine residue. In certain embodiments, the reactive group is attached to an amino-terminal lysine residue. In certain embodiments, the reactive group is attached to a carboxy-terminal lysine residue, for instance, a lysine residue at the carboxy-terminus of GLP-1, GLP-1(7-37) or exendin-4.

The manner of modifying insulinotropic peptides with a reactive group for conjugation to a macromolecule, e.g., albumin, will vary widely, depending upon the nature of the various elements comprising the insulinotropic peptide. The synthetic procedures will be selected so as to be simple, provide for high yields, and allow for a highly purified product. Normally, the chemically reactive group will be created at the last stage of insulinotropic peptide synthesis, for example, with a carboxyl group, esterification to form an active ester. Specific methods for the production of modified insulinotropic peptides are described in U.S. Pat. No. 6,329,336, 6,849,714 or 6,887,849, the contents of each of which are incorporated by reference herein in their entireties.

The insulinotropic peptide conjugates can also be non-specifically conjugated to albumin. Bonds to amino groups will generally be employed, particularly with the formation of amide bonds for non-specific conjugation. To form such bonds, one can use as a chemically reactive group coupled to the insulinotropic peptide a wide variety of active carboxyl groups, particularly esters. While a number of different hydroxyl groups can be employed in these linking agents, the most convenient would be N-hydroxysuccinimide (NHS) and N-hydroxy-sulfosuccinimide (sulfo-NHS). Other linking agents which can be utilized are described in U.S. Pat. No. 5,612,034, which is hereby incorporated by reference herein in its entirety.

In some embodiments, the insulinotropic peptide conjugates can comprise an albumin fusion protein, i.e., an albumin molecule, or a fragment or variant thereof, fused to an insulinotropic peptide. The albumin fusion protein can be generated by translation of a nucleic acid comprising a polynucleotide encoding all or a portion of a therapeutic protein joined to a polynucleotide encoding all or a portion of albumin. In some embodiments, the albumin fusion protein is comprised of albumin, or a fragment or variant thereof, fused to a glucagon-like peptide 1 as described in U.S. Pat. No. 7,141,547 or 7,271,149, which are hereby incorporate by reference in their entireties. In some embodiments, the albumin fusion protein is comprised of albumin, or a fragment or variant thereof, fused to exendin-3, or a fragment or variant thereof. In some embodiments, the albumin fusion protein is comprised of albumin, or a fragment or variant thereof, fused to exendin-4, or a fragment or variant thereof. In some embodiments, the albumin fusion protein is [Gly⁸]GLP-1(7-36)-[Gly⁸]GLP-1(7-36)-human serum albumin (albiglutide) as described in U.S. Pat. No. 7,141,547 or 7,271,149.

5.5.4 Insulinotropic Peptide Synthesis

Insulinotropic peptides can be synthesized by standard methods of solid phase peptide chemistry known to those of ordinary skill in the art. For example, insulinotropic peptides fragments can be synthesized by solid phase chemistry techniques following the procedures described by Steward and Young (Steward, J. M. and Young, J. D., 1984, Solid Phase Peptide Synthesis, 2nd Ed. (Pierce Chemical Company, Rockford, Ill.) using an Applied Biosystem synthesizer. Similarly, multiple fragments can be synthesized then linked together to form larger fragments. These synthetic peptide fragments can also be made with amino acid substitutions at specific locations. For solid phase peptide synthesis, a summary of the many techniques may be found in J. M. Stewart and J. D. Young, 1963, Solid Phase Peptide Synthesis. (W. H. Freeman Co., San Francisco), and J. Meienhofer, 1973, Hormonal Proteins and Peptides, vol. 2, p. 46, Academic Press, New York). For classical solution synthesis see G. Schroder and K. Lupke, The Peptides, Vol. 1, (Academic Press, New York). In some embodiments, synthesis of the insulinotropic peptides is as described in U.S. Pat. No. 6,329,336, 6,849,714 or 6,887,849, the contents of each of which are incorporated by reference herein in their entireties.

5.5.5 Conjugation

Preferably, the peptide and albumin are present in the conjugate in a 1:1 molar ratio, or an approximately 1:1 molar ratio. In a preferred embodiment, the peptide and albumin are present in the conjugate in a 1:1 molar ratio, or an approximately 1:1 molar ratio, and the peptide is attached to the reactive group, optionally through a linking group, at substantially only one site on the peptide and the reactive group is attached to the albumin at substantially only one site on albumin.

Preferably, the albumin in the peptide conjugates is human serum albumin. Preferably, the single site of attachment of the reactive group to albumin is preferably the thiol of cysteine 34 of albumin (e.g., via a maleimide linkage). In a specific embodiment, the reactive group is a single MPA reactive group attached to the peptide, optionally through a linking group, at a single defined amino acid and the MPA is covalently attached to albumin at substantially a single amino acid residue of albumin, preferably cysteine 34.

In a preferred embodiment, a conjugate is formed by contacting a modified peptide comprising a maleimido group with a thiol-containing serum protein, preferably albumin, under conditions comprising a pH of between 3.0 and 8.0, thereby preferably forming a stable thioether linkage which cannot be cleaved under physiological conditions. In preferred embodiments, the serum protein is recombinant human albumin.

In one embodiment, the modified peptide of the conjugate is amidated at its C-terminal end. In another embodiment, the modified peptide is not amidated at its C-terminal end. A conjugate can also comprise such an amidated peptide.

In a preferred embodiment, a single reactive group is covalently attached at a defined site of the modified peptide. In a preferred embodiment of the conjugate, a single reactive group is covalently attached at a defined site of the modified peptide and the reactive group is covalently attached to a single defined site of albumin, preferably to the thiol group of amino acid residue Cys34 of albumin. Preferably, the reactive group of a modified peptide or conjugate of the invention comprises a maleimide group and forms peptide:albumin conjugates of approximately a 1:1 molar ratio. In certain embodiments, a 1:1 molar ratio of peptide to serum protein is preferred over higher ratios because a 1:1 molar ratio provides better biological activity and less immunogenicity than higher ratios (see e.g., Stehle et al. 1997 Anti-Cancer Drugs 8:677-685, incorporated by reference herein in its entirety).

In a preferred embodiment, the albumin is recombinant human albumin. Specific methods for the production of preformed peptide: albumin conjugates are described in U.S. Provisional Application No. 60/791,241, entitled “Process for the Production of Preformed Conjugate of Recombinant Albumin,” filed Apr. 11, 2006, and U.S. patent application Ser. No. 11/645,297 (Publication No. 2007/0269863), entitled “Process for the Production of Preformed Conjugates of Albumin and a Therapeutic Agent,” filed Dec. 22, 2006, the contents of each of which are incorporated by reference herein in their entireties. Specific methods for the purification of peptide: albumin conjugates are described in U.S. Patent Application Publication No. 2005/0267293, which is incorporated by reference herein in its entirety.

In certain embodiments, the conjugate is according to the following:

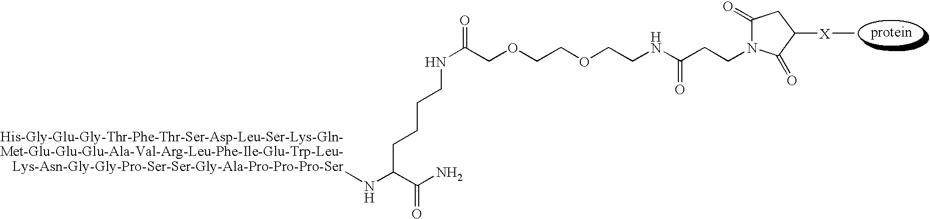

(SEQ ID NO: 31) wherein X is S, O, or NH of an amino acid of said protein. In certain embodiments, said protein is albumin. In certain embodiments, said protein is albumin and X is S (sulfur) of Cys 34 of said albumin. Albumin of the conjugate can be any albumin as described above.

In certain embodiments, the conjugate is according to the following:

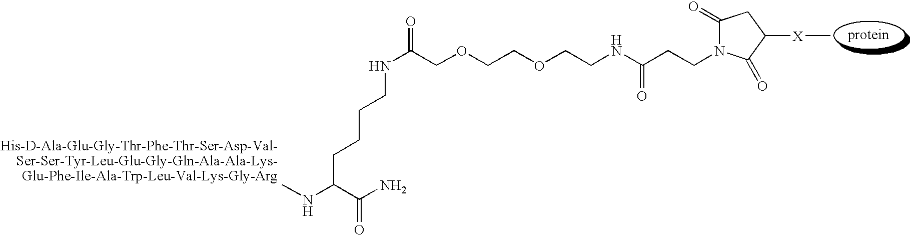

(SEQ ID NO: 32) wherein X is S, O, or NH of an amino acid of said protein. In certain embodiments, said protein is albumin. In certain embodiments, said protein is albumin and X is S (sulfur) of Cys 34 of said albumin. The albumin of the conjugate can be any albumin as described below.

5.5.5.1 Albumin

Any albumin known to those of skill in the art can be used to form a insulinotropic peptide conjugate of the formulations described herein. In some embodiments, the albumin can be serum albumin isolated from a host species and purified for use in the formation of a conjugate. The serum albumin can be any mammalian serum albumin known to those of skill in the art, including but not limited to mouse, rat, rabbit, guinea pig, dog, cat, sheep, bovine, ovine, equine, or human albumin. In some embodiments, the albumin is human serum albumin. In some embodiments, the albumin is bovine serum albumin.

Human serum albumin (HSA) is responsible for a significant proportion of the osmotic pressure of serum and also functions as a carrier of endogenous and exogenous ligands. In its mature form, HSA is a non-glycosylated monomeric protein of 585 amino acids, corresponding to a molecular weight of about 66 kD. Its globular structure is maintained by 17 disulfide bridges which create a sequential series of 9 double loops. See Brown, J. R., Albumin Structure, Function and Uses, Rosenoer, V. M. et al/(eds), Pergamon Press, Oxford (1977), which is incorporated by reference herein in its entirety. The native mature human serum albumin sequence is:

(SEQ ID NO:30)

DAHKSE VAHRFKDLGE ENFKALVLIA FAQYLQQCPF EDHVKLVNEV
TEFAKTCVAD ESAENCDKSL HTLFGDKLCT VATLRETYGE
MADCCAKQEP ERNECFLQHK DDNPNLPRLV RPEVDVMCTA
FHDNEETFLK KYLYEIARRH PYFYAPELLF FAKRYKAAFT
ECCQAADKAA CLLPKLDELR DEGKASSAKQ RLKCASLQKF
GERAFKAWAV ARLSQRFPKA EFAEVSKLVT DLTKVHTECC
HGDLLECADD RADLAKYICE NQDSISSKLK ECCEKPLLEK
SHCIAEVEND EMPADLPSLA ADFVESKDVC KNYAEAKDVF
LGMFLYEYAR RHPDYSVVLL LRLAKTYETT LEKCCAAADP
HECYAKVFDE FKPLVEEPQN LIKQNCELFE QLGEYKFQNA
LLVRYTKKVP QVSTPTLVEV SRNLGKVGSK CCKHPEAKRM
PCAEDYLSVV LNQLCVLHEK TPVSDRVTKC CTESLVNRRP
CFSALEVDET YVPKEFNAET FTFHADICTL SEKERQIKKQ
TALVELVKHK PKATKEQLKA VMDDFAAFVE KCCKADDKET
CFAEEGKKLV AASQAALGL.

Thus, conjugates formed with the mature form of albumin are within the scope of the processes described herein. Unless indicated otherwise, reference to an albumin herein is intended to refer to the mature form of the albumin.

In some embodiments, the albumin is recombinant serum albumin. The recombinant albumin can be any mammalian albumin known to those of skill in the art, including but not limited to mouse, rat, rabbit, guinea pig, dog, cat, sheep, bovine, ovine, equine, or human albumin. In a preferred embodiment, the recombinant albumin is recombinant human albumin, in particular, recombinant human albumin (rHA). In various embodiments, rHA can be produced in a mammalian or non-mammalian organism. In one embodiment, the rHA is produced in a non-mammalian organism. Examples of non-mammalian organisms that can be used for the production of rHA include, without limitation, yeast, bacteria, plants, fungi, and insects. In one embodiment, the rHA is produced in a whole plant or a whole fungus. In another embodiment, the rHA is produced in cultured plant cells, cultured fungus cells, or cultured insect cells. In another embodiment, the rHA is produced in a non-human mammal or in non-human mammalian cells. Examples of non-human mammals that can be used for the production of rHA include, without limitation, those belonging to one of the following: the family Bovidae, the family Canidae, the family Suidae, the order Rodentia, the order Lagomorpha, and the order Primates (excluding humans). In a particular embodiment, the non-human mammal that is used for the production of rHA is selected from the group consisting of a cow, a dog, a pig, a sheep, a goat, a rat, a mouse, a rabbit, a chimpanzee, and a gorilla. In another embodiment, the non-human mammalian cells used for the production of rHA are, without limitation, bovine, canine, porcine, ovine, caprine, rodent, rabbit, or non-human primate cells. The main advantage of rHA produced in a non-human organism compared with albumin purified from human blood or serous fluids is the absence of human-derived products in the manufacturing process of rHA. The use of such controlled production methods leads to a purer product with less structural heterogeneity.

In some embodiments, the insulinotropic peptide conjugate can comprise an albumin precursor. Human albumin is synthesized in liver hepatocytes and then secreted in the blood stream. This synthesis leads, in a first instance, to a precursor, prepro-HSA, which comprises a signal sequence of 18 amino acids directing the nascent polypeptide into the secretory pathway. Thus, conjugates formed with an albumin precursor are within the scope of the conjugates described herein.

In certain embodiments, the insulinotropic peptide conjugate can comprise molecular variants of albumin. Variants of albumin can include natural variants resulting from the polymorphism of albumin in the human population. More than 30 apparently different genetic variants of human serum albumin have been identified by electrophoretic analysis under various conditions. See e.g., Weitkamp et al., Ann. Hum. Genet., 36(4):381-92 (1973); Weitkamp, Isr. J. Med. Sci., 9(9):1238-48 (1973); Fine et al., Biomedicine, 25(8):291-4 (1976); Fine et al., Rev. Fr. Transfus. Immunohematol., 25(2):149-63. (1982); Rochu et al., Rev. Fr. Transfus. Immunohematol. 31(5):725-33 (1988); Arai et al., Proc. Natl. Acad. Sci. USA 86(2): 434-8 (1989), the contents of each of which are incorporated by reference herein in their entireties. Thus, conjugates formed with molecular variants of albumin are within the scope of the conjugates described herein.

In a specific embodiment, the albumin variant has not more than 5, 4, 3, 2 or 1 amino acid substitutions, deletions or insertions relative to the sequence of mature native human serum albumin.

In some embodiments, the insulinotropic peptide conjugate can comprise derivatives of albumin which share substantial homology with albumin. For instance, conjugates can be formed with an albumin homologue having an amino acid sequence which shares at least 75%, at least 80%, at least 85%, more preferably at least 90%, and most preferably at least 95% identity to native human serum albumin, i.e., SEQ ID NO. 30. Percent identity in this context means the percentage of amino acid residues in the candidate sequence that are identical (i.e., the amino acid residues at a given position in the alignment are the same residue) or similar (i.e., the amino acid substitution at a given position in the alignment is a conservative substitution, as discussed above), to the corresponding amino acid residue in the peptide after aligning the sequences and introducing gaps, if necessary, to achieve the maximum percent sequence homology. In certain embodiments, an albumin derivative is characterized by its percent sequence identity or percent sequence similarity with the naturally occurring albumin sequence. Sequence homology, including percentages of sequence identity and similarity, are determined using sequence alignment techniques well-known in the art, preferably computer algorithms designed for this purpose, such as BLAST, using the default parameters of said computer algorithms or the software packages containing them.

In certain embodiments, the albumin homologue comprises a free cysteine. In certain embodiments, the albumin homologue comprises a single free cysteine. In some embodiments, the albumin homologue comprises a free cysteine 34.

In some embodiments, the insulinotropic peptide conjugate can comprise an N-terminal fragment of human serum albumin of at least 100, 200, 300, 400, 500 or more than 500 amino acids. In another embodiment, the insulinotropic peptide conjugate can comprise a human serum albumin variant comprising a modification of the Asp-Ala-His-Lys N-terminal sequence. In another embodiment, the insulinotropic peptide conjugate can comprise at least one deletion among the three N-terminal amino acid residues Asp-Ala-His.

In another embodiment, the insulinotropic peptide conjugate can comprise an N-terminal extension of albumin, such as Glu⁻³, Ala⁻², Glu⁻¹, Phe⁰-HSA (1-585 of SEQ ID NO. 30) or an N-terminal fragment thereof. In another embodiment of the invention the human serum albumin (HSA) variant is selected from the group consisting of HSA (2-585 of SEQ ID NO. 30), HSA (3-585 of SEQ ID NO. 30), HSA (4-585 of SEQ ID NO. 30), Asp-Ala-HSA (4-585 of SEQ ID NO. 30), Xaa³-HSA (1-585 of SEQ ID NO. 30) where Xaa³ is an amino acid residue which has substituted the His residue occupying position 3 in native HSA, and N-terminal fragments thereof.

In some embodiments, the insulinotropic peptide conjugate can comprise structural derivatives of albumin. Structural derivatives of albumin can include proteins or peptides which possess an albumin-type activity, for example, a functional fragment of albumin. In some embodiments, the derivative is an antigenic determinant of albumin, i.e., a portion of a polypeptide that can be recognized by an anti-albumin antibody. In some embodiments, the recombinant albumin can be any protein with preferably a plasma half-life of 75% to 100% of the plasma half-life of human serum albumin in humans and which can be obtained by modification of a gene encoding human serum albumin. By way of example and not limitation, the recombinant albumin can contain insertions or deletions in only the trace metal binding region of albumin, such that binding of trace metals, e.g., nickel and/or copper is reduced or eliminated, as described in U.S. Pat. No. 6,787,636, which is incorporated by reference herein in its entirety. In particular, the recombinant albumin can be modified in the N-terminal region or binding region VI, such as through a truncation of at least one amino acid at the N-terminal end, so that it exhibits reduced or eliminated binding of trace metals such as nickel and/or copper. Other suitable modifications to this binding region include mutations such as an elongation or insertion which will be sufficient to disrupt the trace metal binding which is highest at this site. Reduced trace metal binding by albumin can be advantageous for reducing the likelihood of an allergic reaction to the trace metal in the subject being treated with the albumin composition.

Structural derivatives of albumin can be generated using any method known to those of skill in the art, including but not limited to, oligonucleotide-mediated (site-directed) mutagenesis, alanine scanning, and polymerase chain reaction (PCR) mutagenesis. Site-directed mutagenesis (see Carter, Biochem. J. 237:1-7 (1986); Zoller and Smith, Methods Enzymol. 154:329-50 (1987)), cassette mutagenesis, restriction selection mutagenesis (Wells et al., Gene 34:315-323 (1985)) or other known techniques can be performed on cloned albumin-encoding DNA to produce albumin variant DNA or sequences which encode structural derivatives of albumin (Ausubel et al., Current Protocols In Molecular Biology, John Wiley and Sons, New York (current edition); Sambrook et al., Molecular Cloning, A Laboratory Manual, 3d. ed., Cold Spring Harbor Laboratory Press, Cold Spring Harbor, N.Y. (2001), the contents of each of which are incorporated by reference herein in their entireties.

In certain embodiments, albumin derivatives include any macromolecule with preferably a plasma half-life of 75% to 100% of the plasma half-life of human serum albumin in humans which can be obtained by in vitro modification of the albumin protein. In some embodiments, the albumin is modified with fatty acids. In some embodiments, the albumin is modified with metal ions. In some embodiments, the albumin is modified with small molecules having high affinity to albumin. In some embodiments, the albumin is modified with sugars, including but not limited to, glucose, lactose, mannose, and galactose.

In some embodiments, the insulinotropic peptide conjugate can comprise an albumin fusion protein, i.e., an albumin molecule, or a fragment or variant thereof, fused to a therapeutic protein, or a fragment or variant thereof. The albumin fusion protein can be generated by translation of a nucleic acid comprising a polynucleotide encoding all or a portion of a therapeutic protein joined to a polynucleotide encoding all or a portion of albumin. Any albumin fusion protein known to those of skill in the art can be used to form conjugates according to the processes of the invention. Exemplary albumin fusion proteins are described in U.S. Pat. Nos. 6,548,653, 6,686,179, 6,905,688, 6,994,857, 7,045,318, 7,056,701, 7,141,547 and 7,271,149, the contents of each of which are incorporated by reference herein in their entireties. In some embodiments, the albumin fusion protein is comprised of albumin, or a fragment or variant thereof, fused to a glucagon-like peptide 1 as described in U.S. Pat. No. 7,141,547 or 7,271,149. In some embodiments, the albumin fusion protein is comprised of albumin, or a fragment or variant thereof, fused to exendin-3, or a fragment or variant thereof. In some embodiments, the albumin fusion protein is comprised of albumin, or a fragment or variant thereof, fused to exendin-4, or a fragment or variant thereof. In some embodiments, the albumin fusion protein is comprised of albumin, or a fragment or variant thereof, fused to a multiyear of exendin-4, or a fragment or variant thereof.

Albumin used to form a conjugate described herein can be obtained using methods or materials known to those of skill in the art. For instance, albumin can be obtained from a commercial source, e.g., Novozymes Biopharma UK Ltd. (Nottingham, UK; recombinant human albumin derived from Saccharomyces cerevisiae); Cortex-Biochem (San Leandro, Calif.; serum albumin), Talecris Biotherapeutics (Research Triangle Park, North Carolina; serum albumin), ZLB Behring (King of Prussia, Pa.), or New Century Pharmaceuticals (Huntsville, Ala.; recombinant human albumin derived from Pichia pastoris).

In some embodiments, the albumin is RECOMBUMIN® (Novozymes Biopharma UK Ltd. (Nottingham, UK)). Recombumin® is a recombinant human albumin (rHA) that is produced in vitro using recombinant yeast technology, in which genetically modified yeast (Saccharomyces cerevisiae) secrete soluble rHA which is subsequently harvested, purified and formulated for use as an excipient for the manufacture of biologics or a coating for medical devices. The main advantage of rHA over HSA is that it is expressed in yeast with no animal- or human-derived products used in the manufacturing process. The use of such controlled production methods leads to a purer product with less structural heterogeneity. Previous studies have indicated that there is no significant difference between soluble rHA and HSA in terms of their biochemical characteristics, radiolabelling efficiency and biological behavior in vitro and in vivo. See Dodsworth et al., 1996, Biotechnol. Appl. Biochem. 24: 171-176.

In some embodiments, the albumin is MEDWAY® (ALBREC®, GB-1057, Mitsubishi Tanabe Pharma Corp., Osaka, Japan). MEDWAY is a recombinant human albumin (rHA) that is produced in vitro using recombinant yeast technology, in which genetically modified yeast (Pichia pastoris) secrete soluble rHA which can be subsequently harvested, purified and formulated for the indicated treatment.

In some embodiments, the albumin variant that is used in a conjugate is ALBAGEN™ (New Century Pharma, Huntsville, Ala.). ALBAGEN is HSA (2-585) and is hypoallergenic due to the modified metal binding properties caused by the single N-terminal deletion.

In some embodiments, the albumin is ALBUCULT™ (Novozymes Biopharma UK Ltd. (Nottingham, UK)). Albucult™ is a yeast-derived recombinant human albumin solution designed specifically for cell culture applications. It is produced without the use of animal- or human-derived materials and is therefore free from risk of contaminating human or animal-derived viruses or prions.

### 6. EXAMPLES

The invention is illustrated by the following examples which are not intended to be limiting in any way.

### 6.1 Example 1

### Preparation of Exendin-4 Albumin Conjugates

Exendin-4(1-39) Lys⁴⁰ (ε-AEEA-MPA)-NH₂ conjugated with human serum albumin (HSA) Cys³⁴ (hereinafter “exendin-4(1-39) Lys⁴⁰ (ε-AEEA-MPA)-NH₂ HSA-conjugate” in the following examples) was prepared as described in detail in U.S. Pat. No. 6,329,336; U.S. Pat. Pub. No. 2005/0267293; U.S. patent application Ser. No. 11/645,297, filed Dec. 22, 2006, entitled “Process for the Production of Preformed Conjugate of Recombinant Albumin,” the contents of each of which are incorporated by reference herein in their entireties.

Preparation of Exendin-4(1-39) Lys⁴⁰ (ε-AEEA-MPA)-NH₂

Exendin-4(1-39) Lys⁴⁰ (ε-AEEA-MPA)-NH₂ was prepared according to methods described in U.S. Pat. No. 6,329,336, which is incorporated by reference herein in its entirety. Briefly, solid phase peptide synthesis of Exendin-4 on a 100 μmole scale was performed using manual solid-phase synthesis and a Symphony Peptide Synthesizer using Fmoc protected Rink Amide MBHA resin. The selective deprotection of the Lys(Aloc) group was performed manually and accomplished by treating the resin with a solution of 3 eq of Pd(PPh₃)₄ dissolved in 5 mL of CHCl₃ NMM:HOAc (18:1:0.5) for 2 h. The resin was then washed with CHCl₃ (6×5 mL), 20% HOAc in DCM (6×5 mL), DCM (6×5 mL), and DMF (6×5 mL). The synthesis was then re-automated for the addition of the aminoethoxyethoxyacetic acid (AEEA) group the 3-maleimidopropionic acid (MPA). Resin cleavage and product isolation was performed using 85% TFA/5% TIS/5% thioanisole and 5% phenol, followed by precipitation by dry-ice cold Et₂O. The product was purified by preparative reverse phase HPLC using a Varian (Rainin) preparative binary HPLC system.

Preparation of Exendin-4(1-39) Lys⁴⁰ (ε-AEEA-MPA)-NH—HSA-Conjugates

Exendin-4(1-39) Lys⁴⁰ (ε-AEEA-MPA)-NH₂ was then conjugated to human recombinant serum albumin as described in U.S. patent application Ser. No. 11/645,297 (Publication No. 2007/0269863), filed Dec. 22, 2006, entitled “Process for the Production of Preformed Conjugates of Albumin and a Therapeutic Agent,” the contents of which are incorporated by reference herein in their entirety. Recombinant albumin expressed in Saccharomyces cerevisiae was purified and treated with thioglycolic acid, and purified by phenyl sepharose HIC prior to conjugation. The conjugation reaction comprised 35 μl of 10 mM exendin-4(1-39) Lys⁴⁰ (ε-AEEA-MPA)-NH₂ combined with 175 μl of mercaptalbumin enriched albumin in at a final molar ratio of 0.7:1. The reaction proceeded for 30 minutes at 37° C., and was then stored at 4 C for liquid chromatography/mass spec analysis and purification by butyl sepharose HIC.

Exendin-4(1-39) Lys⁴⁰ (ε-AEEA-MPA)-NH₂ HSA-conjugate was purified by loading the conjugation reaction mixture onto a hydrophobic support equilibrated in aqueous buffer having a high salt content; applying to the support a gradient of decreasing salt concentration; and collecting the eluted albumin conjugate as described in U.S. patent application Ser. No. 11/645,297 (Publication No. 2007/0269863), filed Dec. 22, 2006, entitled “Process for the Production of Preformed Conjugates of Albumin and a Therapeutic Agent,” the contents of which are incorporated by reference herein in their entirety.

### 6.2 Example 2

### Stability Studies on Formulations Comprising Exendin-4(1-39) Lys⁴⁰ (ε-AEEA-MPA)-NH₂ HSA-Conjugates

This example describes formulations which were evaluated and identified as providing suitable conditions and excipients for the preservation of protein structure and stability of exendin-4-albumin conjugates.

6.2.1 Formulation Matrix

Twenty seven formulations were prepared with excipients as shown in Table 1. The exendin-4(1-39) Lys⁴⁰ (ε-AEEA-MPA)-NH₂ HSA-conjugate formulations included (1) a pH range from 5.0 to 7.0 (5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0); (2) 10 mM sodium acetate buffer (pH 5.0) or 10 mM sodium phosphate buffer (pH 6.0-7.0); (3) 150 mM sodium chloride, 5% (w/v) Sorbitol, 9% (w/v) Sucrose or 5% (w/v) Glycerol as a tonicity modifier; (4) 5 mM sodium octanoate, 5 mM sodium octanoate+5 mM Na—N-acetyltryptophan, 5 mM sodium octanoate+5 mM H-Glut, or 5 mM sodium octanoate+20 mM arginine as stabilizers; (5) 0.1% pluronic (w/v) F68 as a surfactant; and (6) an exendin-4(1-39) Lys⁴⁰ (ε-AEEA-MPA)-NH₂ albumin conjugate concentration of 10 mg/mL, 20 mg/mL, or 40 mg/mL.

Stocks of all excipients (sodium acetate, sodium phosphate, sodium chloride, sorbitol, sucrose, glycerol, sodium octanoate, Na—N-acetyltryptophan, H-glut, arginine, plutonic F68), were prepared, sterile filtered and stored at 4° C. Each excipient was added to the final concentration, sterile filtered and the pH of the solution was adjusted. The formulations were packaged for use in sterile 0.5 ml glass vials.

TABLE 1
Formulation Matrix

| Form. ID | Protein Conc. | pH | Buffer | | Tonicity Modifier | | Stabilizer I | Stabilizer II | | Surfactant |
|---|---|---|---|---|---|---|---|---|---|---|
| A5NO | 10 mg/mL | 5 | 10 | mM NaAc | 150 | mM NaCl | 5 mM Octanoate | | | 0.1% F68 |
| A5SO | 10 mg/mL | 5 | 10 | mM NaAc | 5% | Sorbitol | 5 mM Octanoate | | | 0.1% F68 |
| A5SuO | 10 mg/mL | 5 | 10 | mM NaAc | 9% | Sucrose | 5 mM Octanoate | | | 0.1% F68 |
| A5GO | 10 mg/mL | 5 | 10 | mM NaAc | 5% | Glycerol | 5 mM Octanoate | | | 0.1% F68 |
| A5NOG | 10 mg/mL | 5 | 10 | mM NaAc | 150 | mM NaCl | 5 mM Octanoate | 5 | mM H-Glut | 0.1% F68 |
| A5NOR | 10 mg/mL | 5 | 10 | mM NaAc | 150 | mM NaCl | 5 mM Octanoate | 20 | mM R | 0.1% F68 |
| P6NO | 10 mg/mL | 6 | 10 | mM NaPi | 150 | mM NaCl | 5 mM Octanoate | | | 0.1% F68 |
| P6SO | 10 mg/mL | 6 | 10 | mM NaPi | 5% | Sorbitol | 5 mM Octanoate | | | 0.1% F68 |
| P6SuO | 10 mg/mL | 6 | 10 | mM NaPi | 9% | Sucrose | 5 mM Octanoate | | | 0.1% F68 |
| P6GO | 10 mg/mL | 6 | 10 | mM NaPi | 5% | Glycerol | 5 mM Octanoate | | | 0.1% F68 |
| P6NOG | 10 mg/mL | 6 | 10 | mM NaPi | 150 | mM NaCl | 5 mM Octanoate | 5 | mM H-Glut | 0.1% F68 |
| P6NOR | 10 mg/mL | 6 | 10 | mM NaPi | 150 | mM NaCl | 5 mM Octanoate | 50 | mM R | 0,1% F68 |
| P6SOG* | 10 mg/mL | 6 | 10 | mM NaPi | 5% | Sorbitol | 5 mM Octanoate | 5 | mM H-Glut | 0.1% F68 |
| P6SOR | 10 mg/mL | 6 | 10 | mM NaPi | 5% | Sorbitol | 5 mM Octanoate | 20 | mM R | 0.1% F68 |
| P6SA | 10 mg/mL | 6 | 10 | mM NaPi | 5% | Sorbitol | 5 mM Na-N-acetyltryptophane, | | | 0.1% F68 |
| | | | | | | | 5 mM Octanoate | | | |
| 20P6SO | 20 mg/mL | 6 | 10 | mM NaPi | 5% | Sorbitol | 5 mM Octanoate | | | 0.1% F68 |
| 20P6SuO | 20 mg/mL | 6 | 10 | mM NaPi | 9% | Sucrose | 5 mM Octanoate | | | 0.1% F68 |
| 40P6SO | 40 mg/mL | 6 | 10 | mM NaPi | 5% | Sorbitol | 5 mM Octanoate | | | 0.1% F68 |
| 40P6SuO | 40 mg/mL | 6 | 10 | mM NaPi | 9% | Sucrose | 5 mM Octanoate | | | 0.1% F68 |
| P7NO | 10 mg/mL | 7 | 10 | mM NaPi | 150 | mM NaCl | 5 mM Octanoate | | | 0.1% F68 |
| P7SO | 10 mg/mL | 7 | 10 | mM NaPi | 5% | Sorbitol | 5 mM Octanoate | | | 0.1% F68 |
| P7SuO | 10 mg/mL | 7 | 10 | mM NaPi | 9% | Sucrose | 5 mM Octanoate | | | 0.1% F68 |
| P7GO | 10 mg/mL | 7 | 10 | mM NaPi | 5% | Glycerol | 5 mM Octanoate | | | 0.1% F68 |
| P7NOG | 10 mg/mL | 7 | 10 | mM NaPi | 150 | mM NaCl | 5 mM Octanoate | 5 | mM H-Glut | 0.1% F68 |
| P7NOR | 10 mg/mL | 7 | 10 | mM NaPi | 150 | mM NaCl | 5 mM Octanoate | 20 | mM R | 0.1% F68 |

| | | | | | | | | | |
|---|---|---|---|---|---|---|---|---|---|
| *P6NON₂ | 10 mg/mL | 6 | 10 | mM NaPi | 150 | mM NaCl | 5 mM Octanoate | Nitrogen | 0.1% F68 |
| *P6SON₂ | 10 mg/mL | 6 | 10 | mM NaPi | 5% | Sorbitol | 5 mM Octanoate | Nitrogen | 0.1% F68 |

*Nitrogen-blanketed samples.

6.2.2 Methods for Formulation Studies

As summarized in Table 2, several methods were implemented to characterize the physical and chemical stability of the exendin-4(1-39) Lys⁴⁰ (ε-AEEA-MPA)-NH₂ HSA-conjugate in the formulations. Appearance analysis based on visual inspections for clarity, color and the presence of particulates was conducted to determine the quality of the formulations. A pH meter and an osmometer were used to determine maintenance of the pH and osmolality of the formulations within an acceptable range. Peptide concentration analysis by OD₂₈₀ and interaction hydrophobic chromatography (HIC-HPLC) was performed to determine the maintenance of the formulation's peptide concentration within an acceptable range. SDS-PAGE was used to evaluate the purity of peptides in the formulations. Size exclusion chromatograph (SEC-HPLC) was conducted as a test of aggregation, purity and stability in general. Reverse Phase HPLC (RP-HPLC) separates molecules on the basis of relative hydrophobicities and was used to monitor peptide degradants in the formulations.

TABLE 2
Test methods for stability assessment of exendin-4(1-39) Lys⁴⁰
(ε-AEEA-MPA)-NH₂ HSA-conjugate formulations.

| Attribute | Test Method | Time Points | Acceptance Criteria |
|---|---|---|---|
| pH | pH meter | 0, 3, 6 months | 4.0-8.0 |
| Osmolality | osmometer | 0, 3, 6 months | 270-330 mOsm |
| Concentration | HIC-HPLC | 0, 3, 6 months | 9.0-11.0 mg/mL |
| Purity | SDS-PAGE | All | Single band with same |
| | | | MW as standard with |
| | | | absence of large |
| | | | domain degradation |
| Aggregate | SEC-HPLC | All | <1% higher MW |
| Content | | | aggregates |
| Peptide | RP-HPLC | All | |
| Degradants | | | |

The stability of exendin-4(1-39) Lys⁴⁰ (ε-AEEA-MPA)-NH₂ HSA-conjugate in each formulation stored at 4° C., 25° C., and 40° C., for up to six months was examined as summarized in Table 3.

TABLE 3
Stress and time point conditions for CJC-1134-PC candidates.

| | | Time points (months) | | | | | | | | |
|---|---|---|---|---|---|---|---|---|---|---|
| Temperatures | | 0 | 0.25 | 0.50 | 0.75 | 1 | 2 | 3 | 4 | 6 |
| +40° | C. | X | X | X | X | X | X | X | — | — |
| +25° | C. | — | X | X | X | X | X | X | X | X |
| +5° | C. | — | X | X | X | X | X | X | X | X |

6.2.3 pH, Concentration and Osmolality of the Formulations

The pH, conjugate concentration, and osmolality of the formulations were evaluated at time zero; three months at 5° C., 25° C. and 40° C.; and six months at 5° C. and 25° C. as shown in Tables 4 through 9. Formulations comprising glutamic acid, glycerol and arginine were found to be hypertonic and were subsequently removed from the matrix after one month due to instability. Formulations comprising sucrose were removed from the matrix after one month due to redundancy of the nonionic tonicity modifier. Some formulations containing exendin-4(1-39) Lys⁴⁰ (ε-AEEA-MPA)-NH₂ HSA-conjugate at a concentration of 40 mg/ml had less than their target conjugate concentration by more than 2 mg as observed by OD₂₈₀.

TABLE 4
pH, concentration, and osmolality readings at time zero.

| Form. ID | Protein Conc. (mg/mL) | pH | Osmolality |
|---|---|---|---|
| A5NO | 9.3 | 5.23 | 293 |
| A5SO | 9.5 | 5.31 | 289 |
| A5SuO | 9.6 | 5.30 | 286 |
| A5GO | 9.5 | 5.33 | 545 |
| A5NOG | 9.4 | 4.82 | 301 |
| A5NOR | 8.8 | 5.15 | 326 |
| P6NO | 9.2 | 5.94 | 299 |
| P6SO | 9.4 | 6.07 | 293 |
| P6SuO | 9.3 | 6.11 | 291 |
| P6GO | 9.5 | 6.14 | 562 |
| P6NOG | 9.3 | 4.90 | 301 |
| P6NOR | 9.2 | 6.08 | 338 |
| P6SOG | 9.3 | 5.01 | 297 |
| P6SOR | 9.5 | 5.97 | 349 |
| P6SA | No data * | 5.07 | 296 |
| 20P6SO | 17.7 | 6.12 | 289 |
| 20P6SuO | 18.4 | 6.11 | 282 |
| 40P6SO | 34.7 | 6.13 | 302 |
| 40P6SuO | 32.5 | 6.18 | 264 |
| P7NO | 10.0 | 6.55 | 299 |
| P7SO | 9.5 | 6.78 | 297 |
| P7SuO | 9.7 | 6.78 | 289 |
| P7GO | 9.8 | 6.83 | 562 |
| P7NOG | 9.6 | 5.55 | 305 |
| P7NOR | 9.3 | 6.98 | 329 |

* Acetyltryptophan formulation unreadable by spectrophotometer

TABLE 5
pH, concentration, and osmolality for
samples after 3 months at 5° C.

| | Concentration A₂₈₀ | Osmolality | |
|---|---|---|---|
| Formulation | (mg/mL) | (mOsm) | pH |
| A5NO | 10.3 | 291 | 5.25 |
| A5SO | 10.6 | 299 | 5.32 |
| A5NOG | 10.7 | 307 | 4.87 |
| P6NO | 10.1 | 308 | 5.97 |
| P6SO | 10.4 | 304 | 6.07 |
| P6NOG | 10.6 | 321 | 5.73 |
| P6SA | n/a* | 311 | 6.21 |
| 20P6SO | 20.7 | 305 | 6.19 |
| 40P6SO | 37.2 | 310 | 6.25 |
| P7NO | 10.8 | 308 | 6.72 |
| **P6NON₂ | 10.3 | 311 | 6.02 |
| **P6SON₂ | 10.1 | 305 | 6.17 |

*Acetyltryptophan formulation unreadable by spectrophotometer
**Nitrogen-blanketed samples

TABLE 6
pH, concentration, and osmolality readings for
select samples after 3 months at 25° C.

| | Concentration A₂₈₀ | Osmolality | |
|---|---|---|---|
| Formulation | (mg/mL) | (mOsm) | pH |
| A5NO | 10.3 | 295 | 5.26 |
| A5SO | 10.0 | 289 | 5.30 |
| A5NOG | 9.9 | 299 | 4.87 |
| P6NO | 10.1 | 302 | 5.99 |
| P6SO | 9.8 | 290 | 6.08 |
| P6NOG | 10.0 | 308 | 5.70 |
| P6SA | n/a* | 305 | 6.08 |
| 20P6SO | 18.8 | 290 | 6.10 |
| 40P6SO | 37.7 | 306 | 6.16 |
| P7NO | 10.5 | 301 | 6.66 |
| P6NON₂ | 10.0 | 304 | 6.00 |
| P6SON₂ | 9.9 | 293 | 6.06 |

*Acetyltryptophan formulation unreadable by spectrophotometer
**Nitrogen-blanketed samples

TABLE 7
pH, concentration, and osmolality readings for
select samples after 3 months at 40° C.

| | Concentration A₂₈₀ | Osmolality | |
|---|---|---|---|
| Formulation | (mg/mL) | (mOsm) | pH |
| A5NO | 10.2 | 305 | 5.29 |
| A5SO | 10.6 | 293 | 5.30 |
| A5NOG | 9.6 | 301 | 4.84 |
| P6NO | 9.7 | 301 | 6.04 |
| P6SO | 10.1 | 297 | 6.06 |
| P6NOG | 10.0 | 320 | 5.73 |
| P6SA | n/a* | 317 | 6.07 |
| 20P6SO | 20.1 | 309 | 6.06 |
| 40P6SO | 42.8 | 305 | 6.16 |
| P7NO | 10.6 | 307 | 6.66 |

*Acetyltryptophan formulation unreadable by spectrophotometer

TABLE 8
pH, concentration, and osmolality readings for
select samples after 6 months at 5° C.

| | | Osmolality | Concentration |
|---|---|---|---|
| Sample ID | pH | (mOsm) | (mg/mL) |
| A5NO | 5.20 | 307 | 10.0 |
| A5SO | 5.23 | 306 | 10.1 |
| A5NOG | 4.83 | 311 | 10.0 |
| P6NO | 5.89 | 326 | 10.3 |
| P6SO | 6.03 | 313 | 10.4 |
| P6NOG | 5.58 | 339 | 10.3 |
| P6SA | 6.05 | 330 | No data* |
| 20P6SO | 5.98 | 306 | 20.4 |
| 40P6SO | 6.00 | 322 | 39.1 |
| P7NO | 6.51 | 325 | 11.4 |
| P6NO N2 | 5.86 | 326 | 9.6 |
| P6SO N2 | 5.93 | 317 | 10.0 |

*Acetyltryptophan formulation unreadable by spectrophotometer
**Nitrogen-blanketed samples

TABLE 9
pH, Concentration, and Osmolality readings for
select samples after 6 months at 25° C.

| | | Osmolality | Concentration |
|---|---|---|---|
| Sample ID | pH | (mOsm) | (mg/mL) |
| A5NO | 5.14 | 299 | 9.4 |
| A5SO | 5.17 | 292 | 9.7 |
| A5NOG | 4.75 | 307 | 9.6 |
| P6NO | 5.86 | 312 | 9.5 |
| P6SO | 5.90 | 301 | 9.8 |
| P6NOG | 5.55 | 317 | 10.3 |
| P6SA | 5.85 | 318 | No data* |
| 20P6SO | 5.90 | 303 | 19.1 |
| 40P6SO | 5.95 | 326 | 37.0 |
| P7NO | 6.48 | 318 | 10.2 |
| P6NO N2 | 5.86 | 328 | 9.7 |
| P6SO N2 | 5.91 | 310 | 9.6 |

*Acetyltryptophan formulation unreadable by spectrophotometer
**Nitrogen-blanketed samples

6.2.4 Effect of Temperature

The stability profile of exendin-4(1-39) Lys⁴⁵ (ε-AEEA-MPA)-NH₂ HSA-conjugate in different formulations was examined under accelerated stability conditions (temperature at 25° C. or 40° C.) over a period of six months. Major degradation products included peptide degradants and aggregates.

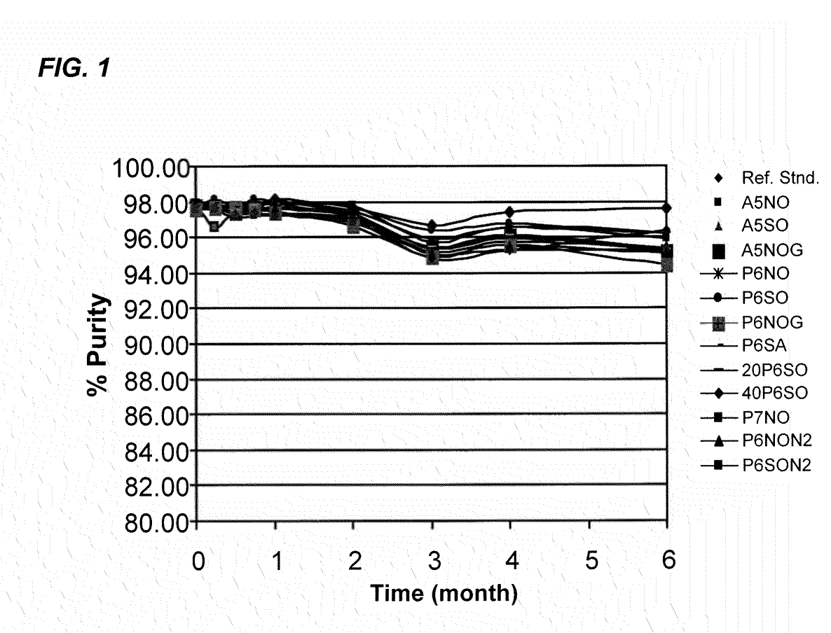

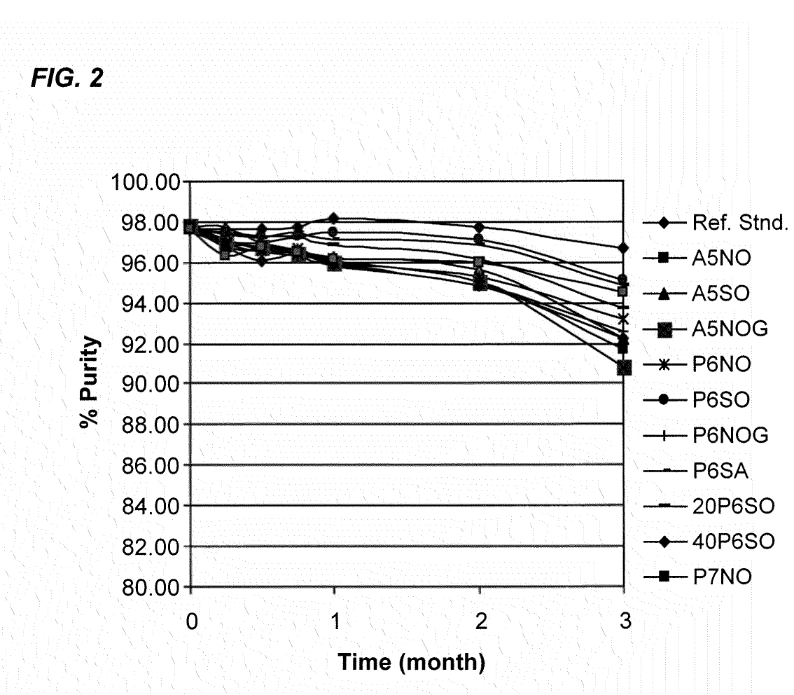

As shown in FIG. 1, sorbitol formulations at pH 6.0 containing either sodium octanoate or a combination of Na—N-acetyltryptophan with sodium octanoate performed slightly better (0.05-0.2%) than other formulations after 6 months at 25° C. Likewise, as shown in FIG. 2, sorbitol formulations at pH 6.0 containing either sodium octanoate or a combination of Na—N-acetyltryptophan with sodium octanoate maintained higher purity (0.4-4.0%) compared to other samples after 3 months at 40° C.

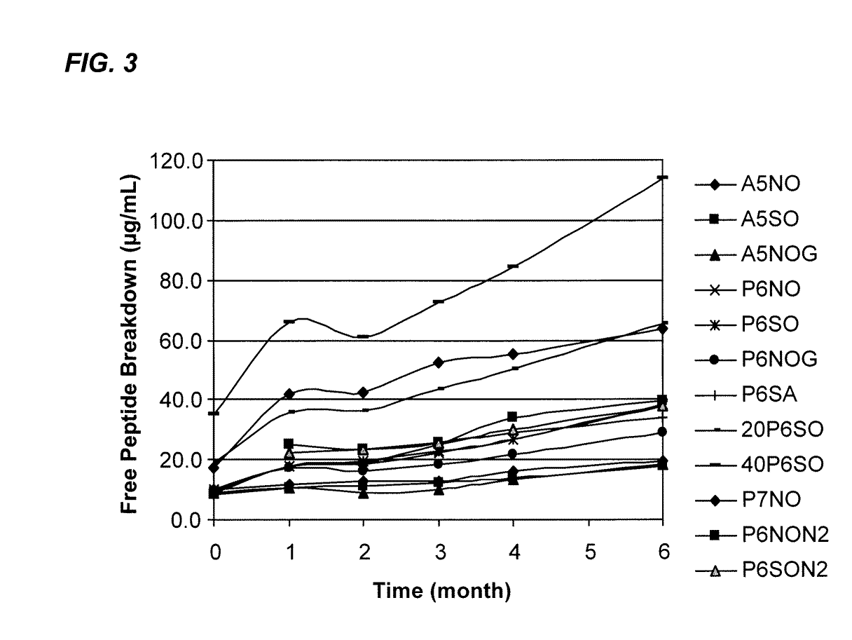

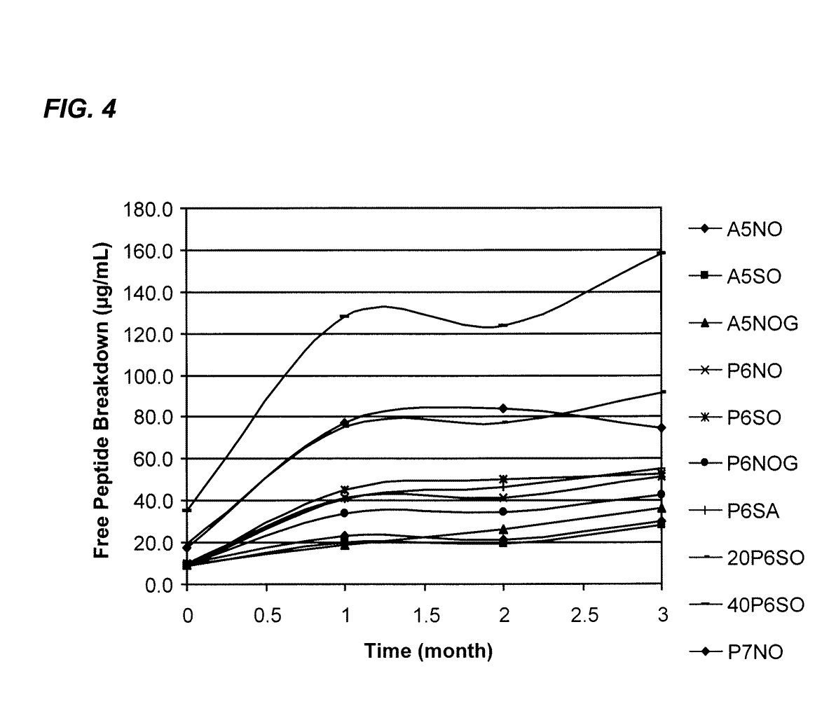

FIGS. 3 and 4 present the time-course of peptide degradants in formulations incubated for 6 months at 25° C., and 3 months at 40° C., respectively, as determined by RP-HPLC. High concentration and high pH formulations, such as formulations with pH 6.0 containing 20 mg/ml or 40 mg/ml exendin-4(1-39) Lys⁴⁰ (ε-AEEA-MPA)-NH₂ HSA-conjugate, as well as formulations with pH 7.0, were found to contain a higher peptide degradants (>20%) than other samples at 25-40° C. Generally, lower pH formulations, such as formulations with pH 5.0, had lower levels of peptide degradants of exendin-4(1-39) Lys⁴⁰ (ε-AEEA-MPA)-NH₂ HSA-conjugate at 40° C.

6.2.5 Effect of Buffers

The stability of exendin-4(1-39) Lys⁴⁰ (ε-AEEA-MPA)-NH₂ HSA-conjugate in sodium acetate buffer and sodium phosphate buffer at 10 mM was tested.

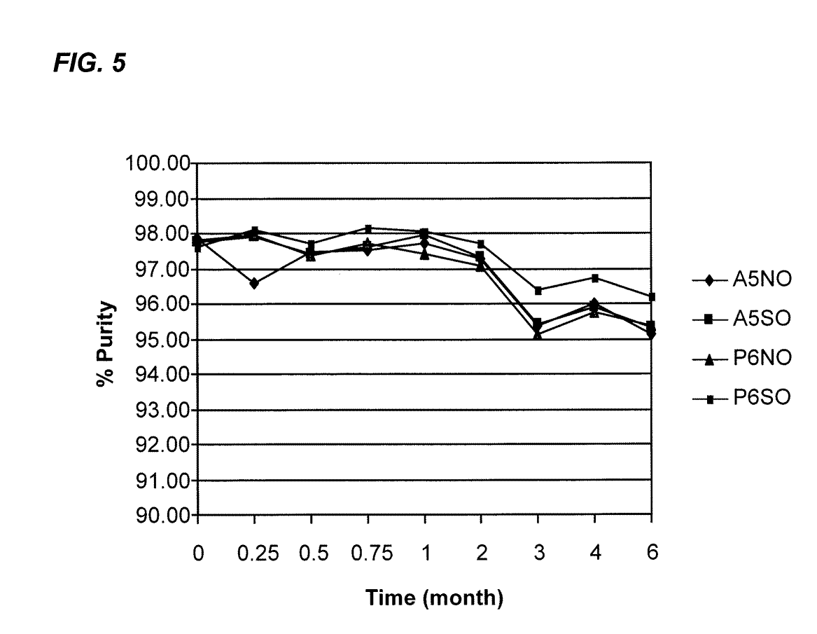

As shown by the SEC-HPLC purity comparison in FIG. 5, the stability of exendin-4(1-39) Lys⁴⁰ (ε-AEEA-MPA)-NH₂ HSA-conjugate in acetate and phosphate buffers did not appear to be significantly different, although formulations containing phosphate buffers performed slightly better after 6 months.

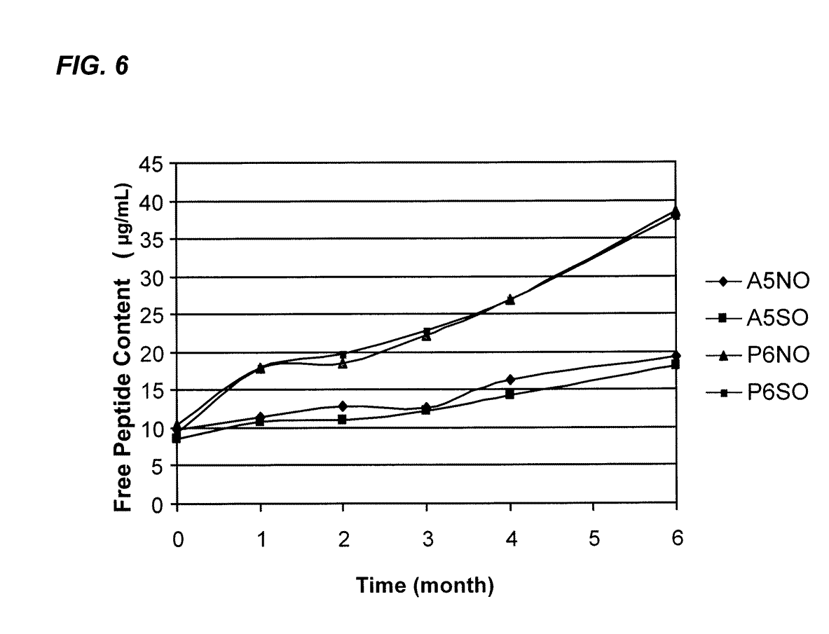

As shown by the RP-HPLC peptide degradants comparison in FIG. 6, a marked increase (>10%) in peptide degradants was observed in sodium phosphate-buffered formulations compared to formulations in sodium acetate buffer at the end of 6 months.

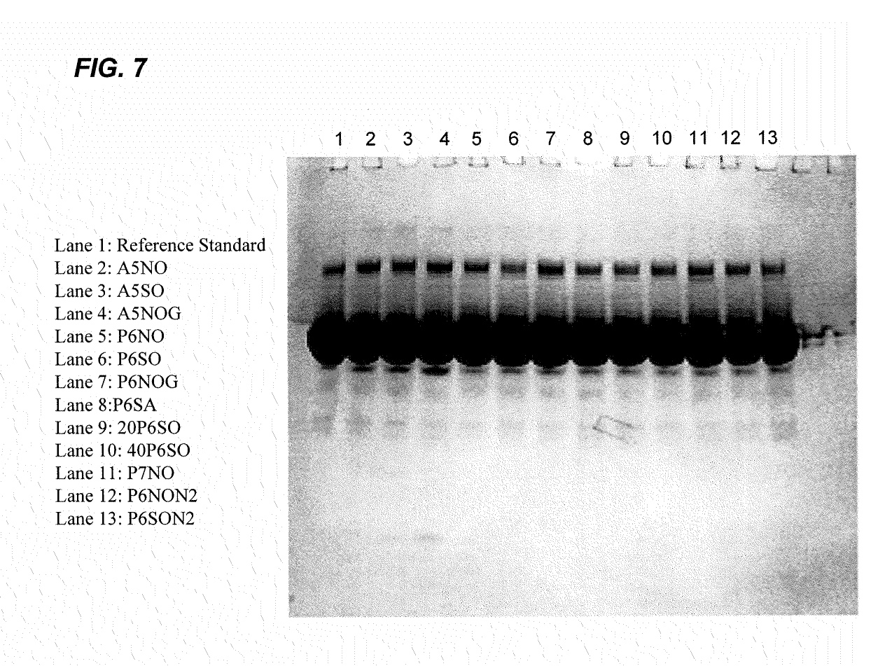

FIG. 7 presents an SDS-PAGE comparison of pH 5.0 formulations in sodium acetate vs. pH 6.0 formulations in sodium phosphate buffers after 6 months at 25° C. Lower pH formulations, such as formulations containing sodium acetate buffer with pH of 5.0, displayed a low molecular weight impurity below the main band and a hint of lower molecular weight degradation product.

6.2.6 Effect of pH

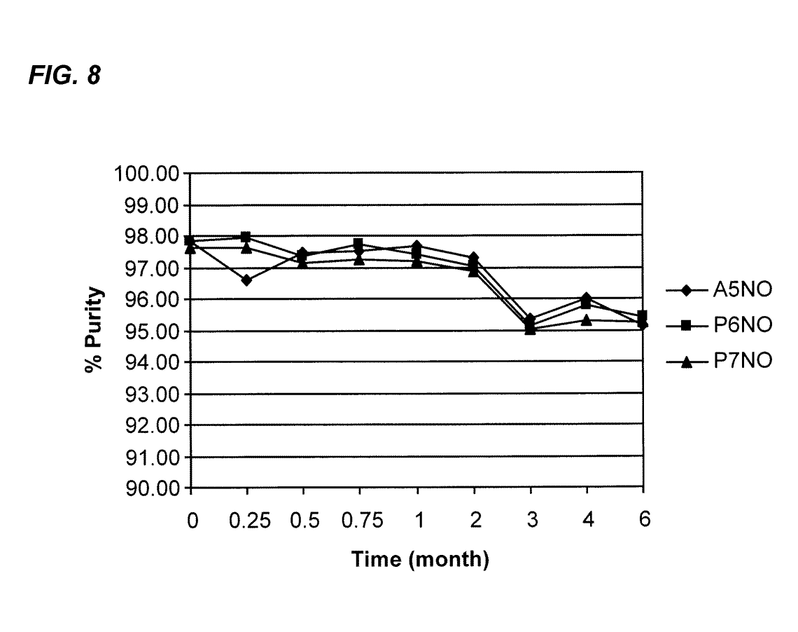

The stability of exendin-4(1-39) Lys⁴⁰ (ε-AEEA-MPA)-NH₂ HSA-conjugate was tested in formulations having a range of pH, including pH 5.0, pH 6.0, and pH 7.0. FIG. 8 presents an SEC-HPLC purity comparison of different pH formulations incubated for 6 months at 25° C. The pH 5.0 and pH 6.0 formulations containing salt performed comparably, with both formulations retaining ˜96.0% purity. At most time points, the pH 7.0 formulation displayed slightly lower purity than the pH 5.0 and pH 6.0 formulations.

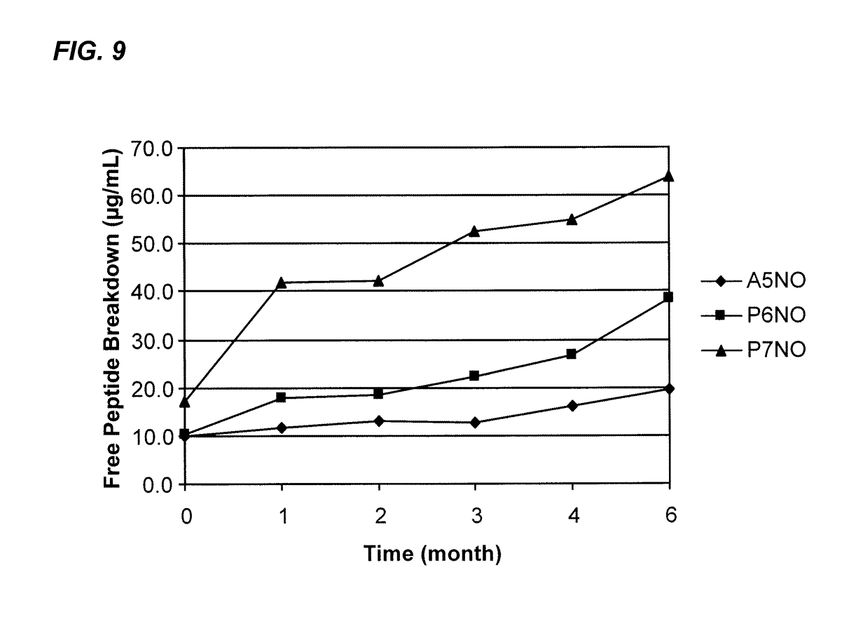

FIG. 9 presents an RP-HPLC peptide degradants comparison of different pH formulations incubated for 6 months at 25° C. The pH 5.0 formulation had the lowest amount of peptide degradants at ˜20 μg/mL; the pH 6.0 formulation had peptide degradants at almost ˜40 μg/mL; and the pH 7.0 formulation had peptide degradants at greater than ˜60 μg/mL.

6.2.7 Effect of Tonicity Modifier

The stability of exendin-4(1-39) Lys⁴⁰ (ε-AEEA-MPA)-NH₂ HSA-conjugate was tested in formulations containing a variety of tonicity modifiers including 150 mM sodium chloride, 5% (w/v) sorbitol, 9% (w/v) sucrose and 5% (w/v) glycerol.

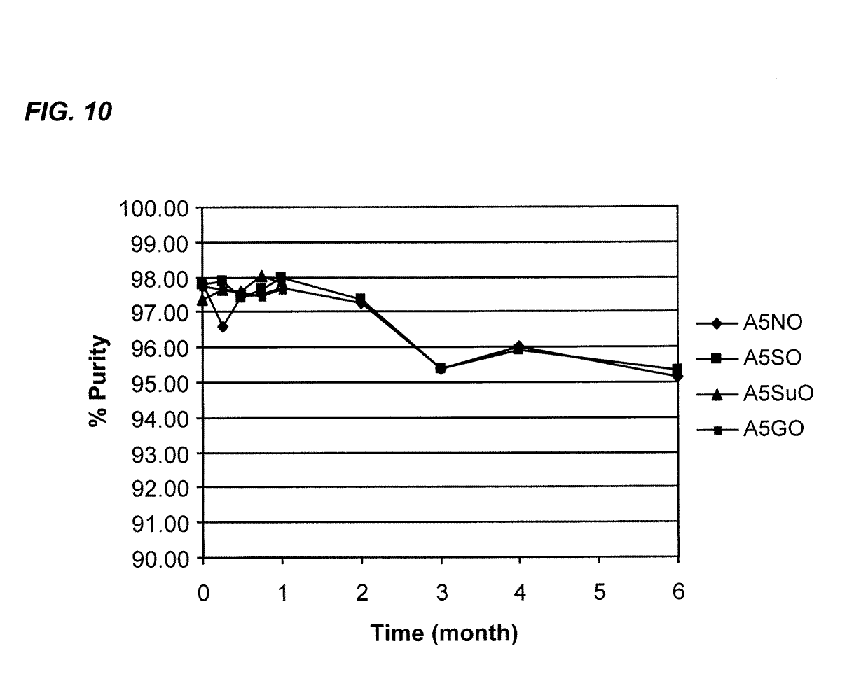

As shown in FIG. 10, which presents an SEC-HPLC purity comparison of pH 5.0 formulations containing different tonicity modifiers incubated for 0-6 months at 25° C., sodium chloride and sorbitol formulations performed comparably (within ˜0.2% purity) after 6 months.

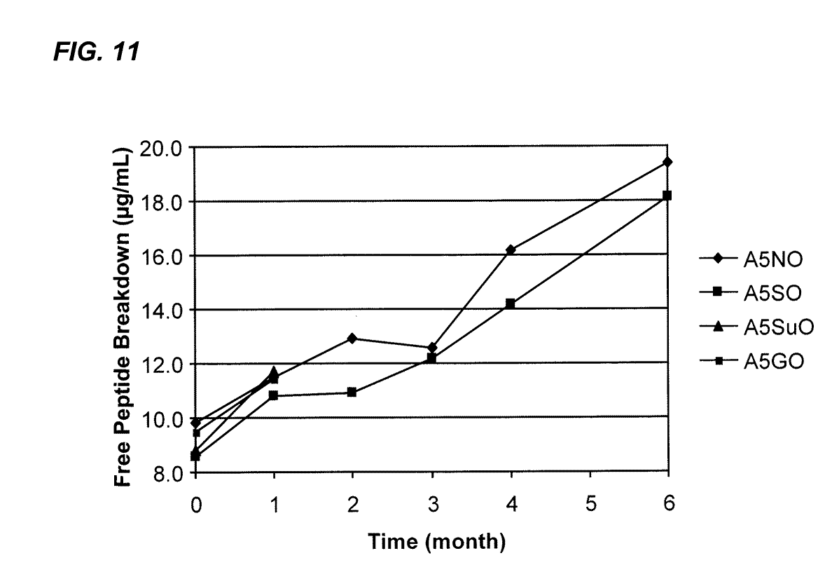

As shown in FIG. 11, which presents an RP-HPLC peptide degradants comparison of pH 5.0 formulations containing different tonicity modifiers incubated for 0-6 months at 25° C., sodium chloride and sorbitol formulations performed comparably after 6 months, with sorbitol formulations containing slightly less (˜10%) peptide degradants than in sodium chloride formulations.

6.2.8 Effect of Stabilizer

A variety of stabilizers were tested in addition to 5 mM sodium octanoate in this study: 5 mM Na—N-acetyltryptophan, 5 mM H-glutamic acid, 20 mM arginine, and nitrogen.

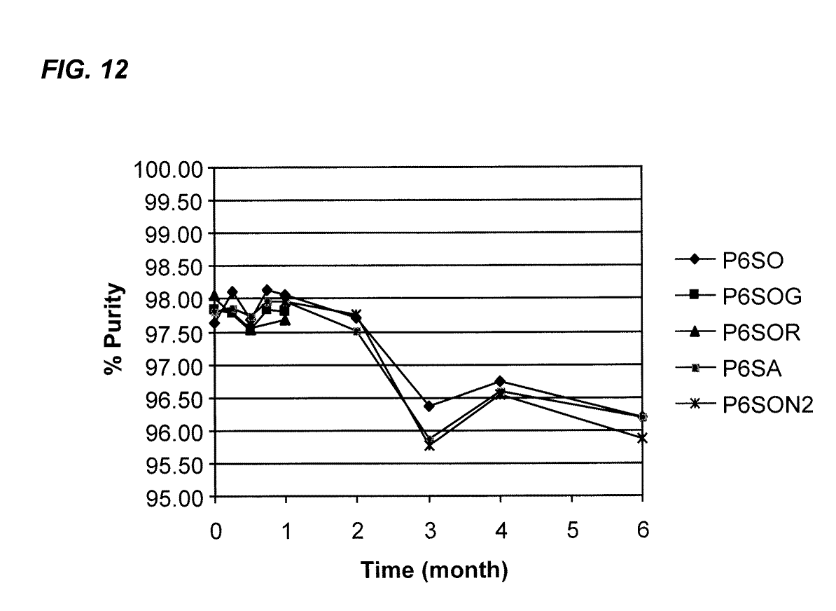

FIG. 12 presents an SEC-HPLC purity comparison of pH 6.0 formulations containing different stabilizers incubated for 0-6 months at 25° C. After 6 months at 25° C., formulations containing 5 mM sodium octanoate, and formulations containing 5 mM sodium octanoate and 20 mM arginine maintained purity at about 96.2%; formulations containing 5 mM sodium octanoate and nitrogen maintained purity at about 95.9%.

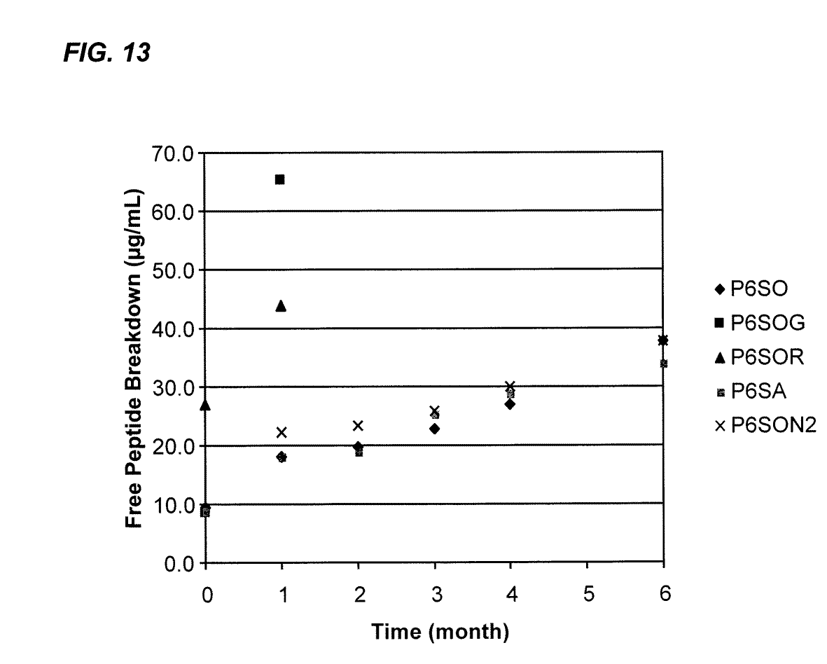

As shown in FIG. 13, which presents an RP-HPLC peptide degradants comparison of pH 6.0 formulations containing different stabilizers incubated for 1-6 months at 25° C., formulations containing 20 mM arginine showed slightly less peptide degradants (˜10%) than formulations containing either 5 mM sodium octanoate or 5 mM sodium octanoate with nitrogen overlay.

6.2.9 Effect of Conjugate Concentration

A range of exendin-4(1-39) Lys⁴⁰ (ε-AEEA-MPA)-NH₂ albumin conjugate concentrations was tested, including 10 mg/ml, 20 mg/ml and 40 mg/ml.

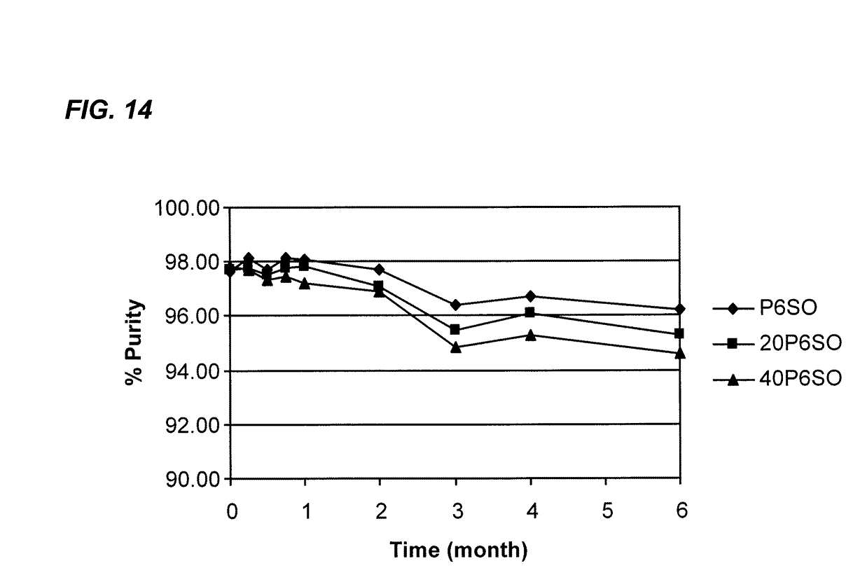

FIG. 14 presents an SEC-HPLC purity comparison of pH 6.0 sorbitol formulations containing 10 mg/ml, 20 mg/ml, and 40 mg/ml of exendin-4(1-39) Lys⁴⁰ (ε-AEEA-MPA)-NH₂ HSA-conjugate when stored for 6 months at 25° C. Purity was observed to be conjugate concentration-dependent. The highest purity was observed in formulation containing 10 mg/ml conjugate, which maintained a level of purity ˜0.9% greater than formulation containing 20 mg/ml conjugate, and ˜1.6% greater purity than formulation containing 40 mg/ml conjugate, following a 6-month incubation at 25° C.

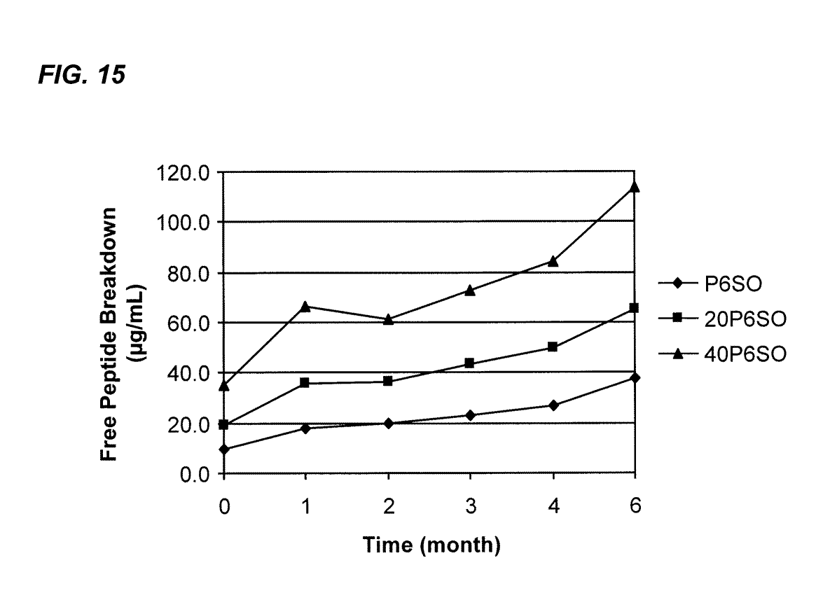

FIG. 15 presents an RP-HPLC purity comparison of pH 6.0 sorbitol formulations containing 10 mg/ml, 20 mg/ml, and 40 mg/ml of CJC-1134-PC following a 6-month incubation at 25° C. Likewise, the amount of peptide degradants was found to be conjugate concentration-dependent, as formulation containing 10 mg/ml conjugate had the lowest amount of peptide degradants at ˜40 μg/mL. Degradation was approximately 1.72-fold higher in the 20 mg/ml formulation and approximately 3-fold higher in the 40 mg/ml formulation relative to the degradation observed in the 10 mg/ml formulation after incubation at 25° C. for 6 months.

### 6.2.10 Conclusion

Peptide degradants appears to be influenced by a combination of buffer composition and pH. Lower pH is preferred for formulations of exendin-4(1-39) Lys⁴⁰ (ε-AEEA-MPA)-NH₂ HSA-conjugate. Both sodium chloride and sorbitol were found to be compatible tonicity modifiers with exendin-4(1-39) Lys⁴⁰ (ε-AEEA-MPA)-NH₂ HSA-conjugate.

SEC-HPLC analysis showed comparable purity data for pH 5.0 and pH 6.0 formulations incubated at higher incubation temperatures, while RP-HPLC showed that the lowest amount of peptide degradants occurred in pH 5.0 formulations. As peptide degradants is considered a more prominent stability issue in exendin-4(1-39) Lys⁴⁰ (ε-AEEA-MPA)-NH₂ HSA-conjugate formulations, a useful pH is pH 5.0 in 10 mM sodium acetate buffer.

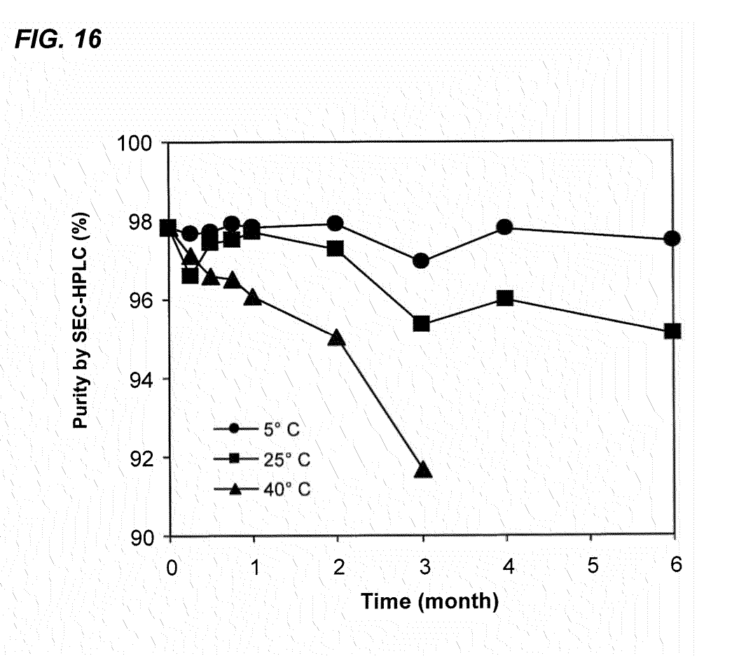

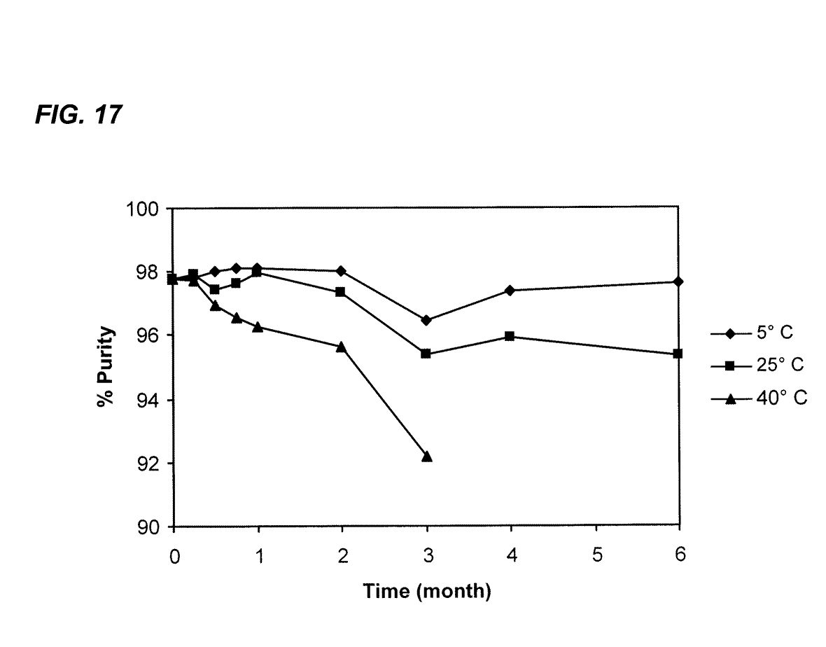

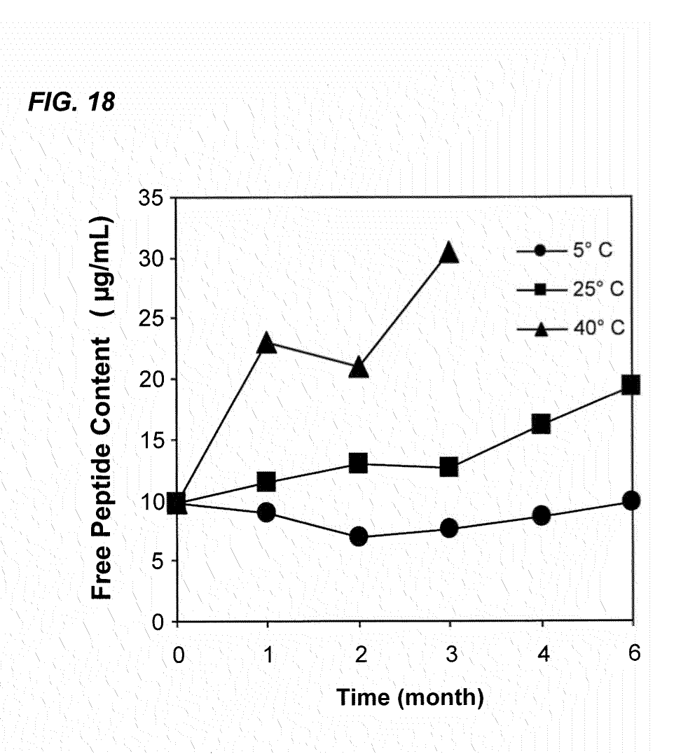

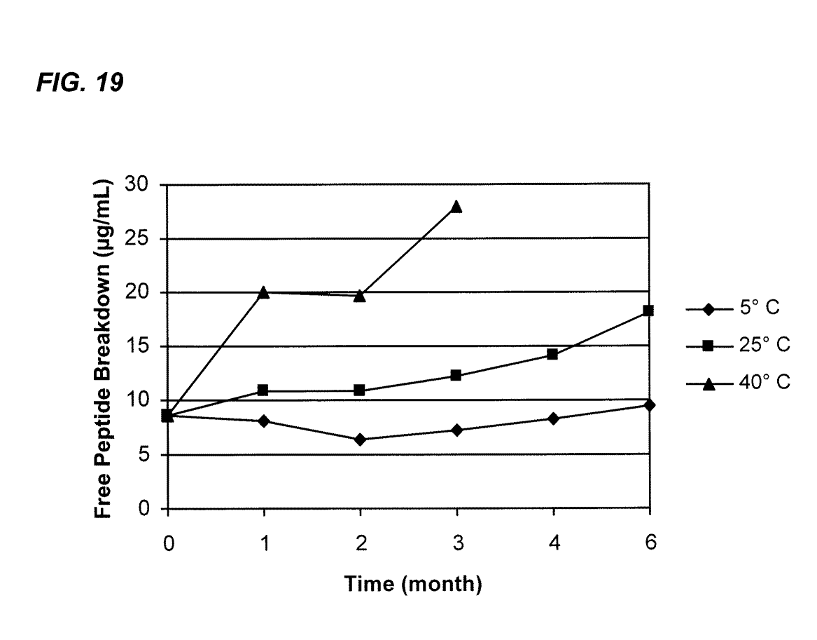

With respect to tonicity modifiers, pH 5.0 formulations containing sodium acetate buffer and either 150 mM sodium chloride or 5% (w/v) sorbitol performed comparably over the course of 6 months when incubated at 4° C., 25° C., and 40° C. SEC-HPLC data showed less than a 0.5% decrease in purity over 6 months at 4° C., and a ˜2.5% decrease at 25° C. for both formulations. After 3 months at 40° C., a ˜5.0% decrease in purity was observed by SEC-HPLC for both formulations. These data are presented in FIG. 16 (150 mM sodium chloride formulation) and FIG. 17 (5% (w/v) sorbitol formulation), respectively. Further, RP-HPLC analysis shows that these two formulations minimized peptide degradants to ˜8-20 μg/mL after 6 months at 4° C. and 25° C., respectively. These data are presented in FIG. 18 (150 mM sodium chloride formulation) and FIG. 19 (5% (w/v) sorbitol formulation), respectively.

Thus, both sodium chloride and sorbitol tonicity modifiers are compatible for formulation with exendin-4(1-39) Lys⁴⁰ (ε-AEEA-MPA)-NH₂ HSA-conjugate. With respect to stabilizer, 5 mM sodium octanoate, as well as the 20 mM arginine formulation maintained purity and a low level of peptide degradants after 6 months at 25° C.

Accordingly, useful formulations include 10 mg/ml exendin-4(1-39) Lys⁴⁰ (ε-AEEA-MPA)-NH₂ HSA-conjugate in 10 mM sodium acetate buffer at pH 5.0, containing 5 mM sodium octanoate, 0.1% (w/v) pluronic F68, and either 150 mM sodium chloride or 5% (w/v) sorbitol.

### 6.3 Example 3

### Preservatives

Various preservatives were examined for their compatibility with the formulations (10 mM sodium phosphate buffer pH 7.0, or 10 mM sodium acetate buffer pH 5.0 with 10 mg/ml exendin-4(1-39) Lys⁴⁰ (ε-AEEA-MPA)-NH₂ HSA-conjugate). Preservative included 0.005%, 0.1%, or 1.0% (w/v) m-cresol, benzyl alcohol, methanol, ethanol, iso-propanol, butyl paraben, ethyl paraben, methyl paraben, phenol, glycerol, xylitol, resorcinol, cathechol, 2,6-dimethylcyclohexanol, 2-methyl-2,4-pentadiol, dextran, polyvinylpyrrolidone, 2-chlorophenol, benzethonium chloride, merthiolate (thimersosal), benzoic acid (propyl paraben) MW 180.2, benzoic acid MW 122.12, benzalkonium chloride, chlorobutanol, sodium benzoate, sodium propionate, and cetylpyridinium chloride.

Formulations containing methanol, ethanol, iso-propanol, glycerol, resorcinol, 2-methyl-2,4-pentadiol, merthiolate (thimerosal), benzalkonium chloride, and sodium benzoate at concentrations of 0.005%, 0.1%, 1.0% (w/v) produced clear solutions. Cetylpyridinium chloride at a concentration of 0.005%, 0.1%, or 1.0% (w/v) produced clear solutions when used in formulations containing sodium phosphate buffer with a pH of 7.0, and produced cloudy solutions when used in formulations containing sodium acetate buffer with a pH of 5.0.

Although butyl paraben, ethyl paraben, or methyl paraben produced clear solutions at concentrations of 0.005% and 0.1% (w/v), each of these preservatives rendered the solutions insoluble at concentrations of 0.3%, 0.5%, 0.7% and 1.0% (w/v).

Similarly, formulations containing m-cresol, benzyl alcohol, phenol, benzethonium chloride, or chlorobutanol were clear at a concentration of 0.1% (w/v), but were opaque, cloudy or not soluble when containing 1% (w/v) of these preservatives.

Formulations containing benzoic acid (propyl paraben) MW 180.2, or benzoic acid MW 122.12 produced clear solutions at a concentration of 0.005% (w/v), but were not soluble at concentrations of 0.1% and 1.0% (w/v) respectively.

This cloudiness or insolubility problem was identified as a potential incompatibility between the buffers (sodium acetate or sodium phosphate), or other components, and the selected preservative in the formulation.

Based on their compatibility with the lead formulations, and safety and frequency of their use, methanol, ethanol, iso-propanol, glycerol, resorcinol, 2-methyl-2,4-pentadiol, merthiolate (thimerosal), benzalkonium chloride, sodium benzoate, and cetylpyridinium chloride are useful preservatives in exendin-4(1-39) Lys⁴⁰ (ε-AEEA-MPA)-NH₂ albumin conjugate formulations.

### 6.4 Example 4

### Stability of Exendin-4(1-39) Lys⁴⁰ (ε-AEEA-MPA)-NH₂ HSA-Conjugate in 10 mM Sodium Acetate Buffer at pH 5.0, 5 mM Sodium Octanoate, 0.1% (w/v) Pluronic F68 and 150 mM NaCl

This example demonstrates the stability of exendin-4(1-39) Lys⁴⁰ (ε-AEEA-MPA)-NH₂ HSA-conjugate formulated in 10 mM sodium acetate buffer at pH 5.0, 5 mM sodium octanoate, 0.1% (w/v) pluronic F68, and 150 mM sodium chloride when incubated at 5° C., 25° C. (for up to 12 months) and 40° C. (for up to 3 months).

Stocks of all excipients (sodium acetate, sodium chloride, octanoate, pluronic F68), were prepared, sterile filtered and stored at 4° C. Each excipient was added to the final concentration, sterile filtered and the pH of the solution was adjusted. The formulations were packaged for use in sterile 3.0 ml Type I glass vials with 13 mm gray butyl stoppers.

Stability of the of exendin-4(1-39) Lys⁴⁰ (ε-AEEA-MPA)-NH₂ HSA-conjugate was determined by measuring: (1) visual appearance; (2) pH, as measured by pH meter; (3) protein concentration, as measured by HIC-HPLC and A₂₈₀; (4) purity, as determined by SDS-PAGE; (5) the amount of peptide degradants, as measured by RP-HPLC; and (6) the aggregate content (species comprising >trimers) as measured by SEC-HPLC.

Results of the stability study are presented in Tables 10-12. The stability of exendin-4(1-39) Lys⁴⁰ (ε-AEEA-MPA)-NH₂ HSA-conjugate formulated in 10 mM sodium acetate buffer at pH 5.0, 5 mM sodium octanoate, 0.1% (w/v) pluronic F68, and 150 mM was maintained for at least 12 months when incubated at 5° C. and 25° C., and for at least 3 months when incubated at 40° C. At each time point, the formulation displayed a clear, straw to amber colored appearance which was free from particulates; the pH was maintained between 4.5 and 6.0; protein concentration was maintained between 8.0 and 12 mg/mL; following SDS-PAGE, a single band appeared, consistent in molecular weight with a conjugate standard and showing no large domain degradation; and higher molecular weight aggregate content was <1%.

TABLE 10
Stability of Exendin-4 HSA-Conjugate (sodium acetate buffer, pH 5.0 formulation) Stored at 5 ± 3° C.

| | Initial | 1 Month | 2 Months | 3 Months | 6 Months | 9 Months | 12 Months |
|---|---|---|---|---|---|---|---|
| Appearance | Clear | Clear | Clear | Clear | Clear | Clear | Clear |
| pH | 5.1 | 5.0 | 4.9 | 5.0 | 5.0 | 5.0 | 4.8 |
| Assay (HIC) | 11.6 | n/s | n/s | n/s | 11.3 | n/s | 10.8 |
| (mg/mL) | | | | | | | |
| Assay (A₂₈₀) | 9.3 | 9.5 | 9.7 | 9.4 | 9.4 | 10.4 | 9.9 |
| (mg/mL) | | | | | | | |
| Purity* | Single band | Single band | Single band | Single band | Single band | Single band | Single band |
| Peptide | 1.3 | 1.8 | 2.0 | 2.4 | 2.1 | 2.7 | 2.9 |
| Degradants | | | | | | | |
| (μg/mL) | | | | | | | |
| Aggregate | 0.1 | 0.1 | 0.1 | 0.2 | 0.1 | 0.1 | 0 |
| Content (%) | | | | | | | |

*as determined by SDS-PAGE

TABLE 11
Stability of Exendin-4 HSA-Conjugate (sodium acetate buffer, pH 5.0 formulation) Stored at 25 ± 2° C.

| | Initial | 1 Month | 2 Months | 3 Months | 6 Months | 9 Months | 12 Months |
|---|---|---|---|---|---|---|---|
| Appearance | Clear | Clear | Clear | Clear | Clear | Clear | Clear |
| pH | 5.1 | 5.0 | 4.9 | 5.1 | 5.1 | 5.1 | 4.9 |
| Assay (HIC) | 11.6 | n/s | n/s | n/s | 10.7 | n/s | 8.8 |
| (mg/mL) | | | | | | | |
| Assay (A₂₈₀) | 9.3 | 9.4 | 9.6 | 9.6 | 9.6 | 9.9 | 9.9 |
| (mg/mL) | | | | | | | |
| Purity* | Single band | Single band | Single band | Single band | Single band | Single band | Single band |
| Peptide | 1.3 | 5.3 | 7.8 | 9.3 | 13.3 | 16.0 | 16.5 |
| Degradants | | | | | | | |
| (μg/mL) | | | | | | | |
| Aggregate | 0.1 | 0.1 | 0.1 | 0.1 | 0.1 | 0.1 | 0.1 |
| Content (%) | | | | | | | |

*as determined by SDS-PAGE

TABLE 12
Stability of Exendin-4 HSA-Conjugate (sodium acetate buffer,
pH 5.0 formulation) Stored at 40 ± 2° C.

| ATTRIBUTE | Initial | 0.5 Month | 1 Month | 3 Months |
|---|---|---|---|---|
| Appearance | Clear | Clear | Clear | Clear |
| pH | 5.1 | 5.0 | 5.0 | 5.0 |
| Assay (HIC) | 11.6 | n/s | n/s | n/s |
| (mg/mL) | | | | |
| Assay (A₂₈₀) | 9.3 | 9.9 | 9.5 | 9.6 |
| (mg/mL) | | | | |
| Purity* | Single | Single | Single | Single |
| | band | band | band | band |
| Peptide Degradants | 1.3 | 12.5 | 18.5 | 25.9 |
| (μg/mL) | | | | |
| Aggregate Content | 0.1 | 0.1 | 0.1 | 0.2 |
| (%) | | | | |

*as determined by SDS-PAGE

### 6.5 Example 5

### Stability of Exendin-4(1-39) Lys⁴⁰ (ε-AEEA-MPA)-NH₂ HSA-Conjugate in 10 mM Sodium Phosphate Buffer at pH 7.0, 1.6 mM Sodium Octanoate, 15 mg/L Polysorbate 80 and 135 mM Sodium Chloride

This example demonstrates the stability of exendin-4(1-39) Lys⁴⁰ (ε-AEEA-MPA)-NH₂ HSA-conjugate formulated in 10 mM sodium phosphate buffer at pH 7.0, 1.6 mM sodium octanoate, 15 mg/L polysorbate 80 and 135 mM sodium chloride when incubated at 5° C., 25° C. (for up to 18 months) and 40° C. (for up to 6 months).

Stocks of all excipients (sodium phosphate, sodium chloride, sodium octanoate, polysorbate 80), were prepared, sterile filtered and stored at 4° C. Each excipient was added to the final concentration, sterile filtered and the pH of the solution was adjusted. The formulations were packaged for use in sterile 3.0 ml Type I glass vials with 13 mm gray butyl stoppers.

Stability of the of exendin-4(1-39) Lys⁴⁰ (ε-AEEA-MPA)-NH₂ HSA-conjugate was determined by measuring: (1) visual appearance; (2) pH, as measured by pH meter; (3) osmolality (mOsm), as measured by osmometer; (4) purity, as determined by SDS-PAGE; (5) the amount of peptide degradants, as measured by RP-HPLC; and (6) the aggregate content (species comprising >trimers) as measured by SEC-HPLC.

Results of the stability study are presented in Tables 13-15. At each time point, the formulation displayed a clear, straw to amber colored appearance which was free from particulates; the pH was maintained at 7.0; osmolality was maintained between 250-330 mOsm; following SDS-PAGE, a single band appeared, consistent in molecular weight with a conjugate standard and showing no large domain degradation; and higher molecular weight aggregate content was 0%.

TABLE 13
Stability of Exendin-4 HSA-Conjugate (sodium phosphate buffer, pH 7.0 formulation) Stored at 5 ± 3° C.

| | Initial | 1 Month | 3 Months | 6 Months | 9 Months | 12 Months | 18 Months |
|---|---|---|---|---|---|---|---|
| Appearance | Clear | Clear | Clear | Clear | Clear | Clear | Clear |
| pH | 7 | 7 | 7 | 7 | 7 | 7 | 7 |
| Osmolality | 276 | 274 | 278 | 280 | 281 | 276 | 272 |
| (mOsm) | | | | | | | |
| Purity* | Single band | Single band | Single band | Single band | Single band | Single band | Single band |
| Peptide | 45 | 39 | 47 | 42 | 44 | 53 | 69 |
| Degradants | | | | | | | |
| (μg/mL) | | | | | | | |
| Aggregate | 0 | 0 | 0 | 0 | 0 | 0 | 0 |
| Content (%) | | | | | | | |

*as determined by SDS-PAGE

TABLE 14
Stability of Exendin-4 HSA-Conjugate (sodium phosphate buffer, pH 7.0 formulation) Stored at 25 ± 2° C.

| | Initial | 1 Month | 3 Months | 6 Months | 9 Months | 12 Months | 18 Months |
|---|---|---|---|---|---|---|---|
| Appearance | Clear | Clear | Clear | Clear | Clear | Clear | Clear |
| pH | 7 | 7 | 7 | 7 | 7 | 7 | 7 |
| Osmolality | 276 | 275 | 281 | 280 | 285 | 279 | 279 |
| (mOsm) | | | | | | | |
| Purity* | Single band | Single band | Single band | Single band | Single band | Single band | Single band |
| Peptide | 45 | 78 | 127 | 119 | 96 | 93 | 153 |
| Degradants | | | | | | | |
| (μg/mL) | | | | | | | |
| Aggregate | 0 | 0 | 0 | 0 | 0 | 0 | 0 |
| Content (%) | | | | | | | |

*as determined by SDS-PAGE

TABLE 15
Stability of Exendin-4 HSA-Conjugate (sodium phosphate buffer,
pH 7.0 formulation) Stored at 40 ± 2° C.

| ATTRIBUTE | Initial | 1 Month | 3 Month | 6 Months |
|---|---|---|---|---|
| Appearance | Clear | Clear | Clear | Clear |
| pH | 7 | 7 | 7 | 7 |
| Osmolality (mOsm) | 276 | 276 | 285 | 282 |
| Purity* | Single | Single | Single | Single |
| | band | band | band | band |
| Peptide Dcgradants | 45 | 55 | 119 | 86** |
| (μg/mL) | | | | |
| Aggregate Content | 0 | 0 | 0 | 0 |
| (%) | | | | |

*as determined by SDS-PAGE
**many peaks below level of quantitation (15 μg/ml) not included in the total

### 6.6 Example 6

### Effect of an Exendin-4 Conjugate Formulation on Blood Glucose Levels

This example describes the results of a randomized, placebo-controlled, double-blind single escalating dose Phase I/II clinical study to evaluate the safety, tolerability, pharmacokinetics and pharmacodynamic effect of a range of doses of an exendin-4(1-39) Lys⁴⁰ (ε-AEEA-MPA)-NH₂ HSA-conjugate formulation administered subcutaneously to subjects with Type II diabetes mellitus.

The effects of four single subcutaneous doses (including 1.5 mg and 2.0 mg) of exendin-4(1-39) Lys⁴⁰ (ε-AEEA-MPA)-NH₂ HSA-conjugate and placebo were studied. The conjugate was administered at a concentration of 10 mg/ml in a formulation described herein.

Fasting plasma glucose levels were determined from days 2 through 7 for each subject following dosing with exendin-4(1-39) Lys⁴⁰ (ε-AEEA-MPA)-NH₂ HSA-conjugate. Blood glucose levels were also measured using a glucometer at six timepoints per day: (1) fasting/5 minutes before starting breakfast; (2) 2 hours after starting breakfast; (3) 5 minutes prior to starting lunch; (4) 2 hours after starting lunch; (5) 5 minutes before starting dinner; and (6) 2 hours after starting dinner. For each subject, the mean value of these six measurements was calculated for days 1-7 following dosing.

Fasting plasma glucose levels and mean daily glucose levels in the conjugate treated subjects were reduced in comparison to fasting plasma glucose levels and mean daily glucose levels, respectively, in the placebo treated subjects.

### 6.7 Example 7

### Treatment of Type II Diabetes with an Exendin-4(1-39) Lys⁴⁰ (ε-AEEA-MPA)-NH₂ HSA-Conjugate Formulation

A pharmaceutical formulation comprising 10 mg/ml exendin-4(1-39) Lys⁴⁰ (ε-AEEA-MPA)-NH₂ HSA-conjugate in 10 mM sodium acetate buffer at pH 5.0, containing 5 mM sodium octanoate, 0.1% (w/v) pluronic F68 and 150 mM sodium chloride is used to treat Type II diabetes in a human subject in need thereof. Patients with Type II diabetes receive either: (1) a once-a-week dose of the formulation comprising 1.5 mg of the exendin-4(1-39) Lys⁴⁰ (ε-AEEA-MPA)-NH₂ HSA-conjugate for a total 12-week treatment; or (2) a once-a-week dose of the formulation comprising 1.5 mg of exendin-4(1-39) Lys⁴⁰ (ε-AEEA-MPA)-NH₂ HSA-conjugate for four weeks, followed by a once-a-week dose of the formulation comprising 2.0 mg of exendin-4(1-39) Lys⁴⁰ (ε-AEEA-MPA)-NH₂ HSA-conjugate for eight weeks.

Patients are on a stable dose of >1000 mg metformin daily for at least 3 months prior to treatment with the conjugate. Subjects undergo a routine screening evaluation up to 14 days prior to the first administration of the conjugate. Patients who have been diagnosed with Type II diabetes mellitus at least 3 months prior to screening are assessed for the following criteria: informed consent; complete medical history; review of inclusion/exclusion criteria; survey of concomitant medications; complete physical examination; body weight; vital signs (blood pressure, temperature, pulse, respiratory rate); 12-lead ECG, urine drug screen and alcohol breath test; clinical laboratory analysis (clinical chemistry, hematology, and coagulation); urinalysis; serum pregnancy test (for pre-menopausal females only); fasting plasma glucose; HbA1c level; fructosamine, lipid profile; total IgE level; and immunogenicity sampling.

The exendin-4(1-39) Lys⁴⁰ (ε-AEEA-MPA)-NH₂ HSA-conjugate is administered by subcutaneous injection in the abdomen of the patient in a fasting state in the early morning. Patients are monitored throughout the dosing period by a practitioner of skill in the art, including blood sampling for clinical laboratory analysis (clinical chemistry, hematology, coagulation), fructosamine, lipid profile, and HbA1c; 12-lead ECG; and physical examination to determine the safety and effectiveness of the exendin-4(1-39) Lys⁴⁰ (ε-AEEA-MPA)-NH₂ HSA-conjugate formulation.

### 6.8 Example 8

### Treatment of Type II Diabetes with an Exendin-4(1-39) Lys⁴⁰ (ε-AEEA-MPA)-NH₂ HSA-Conjugate Formulation

A pharmaceutical formulation comprising 10 mg/ml exendin-4(1-39) Lys⁴⁰ (ε-AEEA-MPA)-NH₂ HSA-conjugate in 10 mM sodium acetate buffer at pH 5.0, containing 5 mM sodium octanoate, 0.1% (w/v) pluronic F68 and 150 mM sodium chloride is used to treat Type II diabetes in a human subject in need thereof. Patients with Type II diabetes receive either: (1) a twice-a-week dose of the formulation comprising 1.5 mg exendin-4(1-39) Lys⁴⁰ (ε-AEEA-MPA)-NH₂ HSA-conjugate, for a total weekly dose of the conjugate of 3.0 mg, for 12 weeks of treatment; or (2) a twice-a-week dose of the formulation comprising 1.5 mg exendin-4(1-39) Lys⁴⁰ (ε-AEEA-MPA)-NH₂ HSA-conjugate, for a total weekly dose of the conjugate of 3.0 mg, for 4 weeks of treatment, followed by a once-a-week dose of the formulation comprising 2.0 mg of exendin-4(1-39) Lys⁴⁰ (ε-AEEA-MPA)-NH₂ HSA-conjugate for eight additional weeks of treatment.

Patients are on a stable dose of ≧1000 mg metformin daily for at least 3 months prior to treatment with the conjugate. Subjects undergo a routine screening evaluation up to 14 days prior to the first administration of the conjugate. Patients who have been diagnosed with Type II diabetes mellitus at least 3 months prior to screening are assessed for the following criteria: informed consent; complete medical history; review of inclusion/exclusion criteria; survey of concomitant medications; complete physical examination; body weight; vital signs (blood pressure, temperature, pulse, respiratory rate); 12-lead ECG, urine drug screen and alcohol breath test; clinical laboratory analysis (clinical chemistry, hematology, and coagulation); urinalysis; serum pregnancy test (for pre-menopausal females only); fasting plasma glucose; HbA1c level; fructosamine, lipid profile; total IgE level; and immunogenicity sampling.

The exendin-4(1-39) Lys⁴⁰ (ε-AEEA-MPA)-NH₂ HSA-conjugate is administered by subcutaneous injection in the abdomen of the patient in a fasting state in the early morning. Patients are monitored throughout the dosing period by a practitioner of skill in the art, including blood sampling for clinical laboratory analysis (clinical chemistry, hematology, coagulation), fructosamine, lipid profile, and HbA1c; 12-lead ECG; and physical examination to determine the safety and effectiveness of the exendin-4(1-39) Lys⁴⁰ (ε-AEEA-MPA)-NH₂ HSA-conjugate formulation.

### 6.9 Example 9

### Subjects Treated with an Exendin-4(1-39) Lys⁴⁰ (ε-AEEA-MPA)-NH₂ HSA-Conjugate Formulation as Described in Examples 7 and 8

A first clinical trial comprising the dosing regimen described in Example 7 was conducted. The trial lasted for three months and enrolled 144 patients having type II diabetes not adequately controlled by metformin therapy. Patients were randomized to one of three parallel treatment groups: a 1.5 mg per week cohort; a 1.5 mg per week cohort titrating to 2 mg per week after four weeks; and a placebo cohort. A second clinical trial comprising the dosing regimen described in Example 8 was also conducted. The trial lasted for three months and enrolled 80 patients having type II diabetes not adequately controlled by metformin therapy. Patients were randomized to one of three parallel treatment groups: a 1.5 mg twice-weekly cohort titrating to 2 mg per week after four weeks; a 3 mg (1.5 mg twice per week) cohort; and a placebo cohort. The two trials had the same entry criteria and study assessments, thus allowing an integrated analysis.

The conjugate of the formulation was manufactured using Recombumin®, which is recombinant albumin produced by Novozymes Biopharma. The pharmaceutical formulation was injected as a small volume (≦0.2 ml) with a 31 gauge needle.

In the treatment of diabetes, the primary demonstration of efficacy of an anti-diabetic agent is reduction of HbA1c. HbA1c % (percentage of hemoglobin A1c, i.e., glycosylated hemoglobin) is representative of the average blood glucose level of a subject during the months preceding treatment with an anti-diabetic agent, and is the most commonly used measure of chronic glycemia.

Significant reductions in HbA1c were seen throughout the treatment period in all active treatment groups compared to both baseline and placebo groups (1.5 mg, 2 mg combined arms, and 3 mg per protocol by integrated analysis). The most robust reduction was observed in the 3 mg dose group in which patients achieved a HbA1c decrease of 1.4% at the end of the 12 week treatment period. The HbA1c reduction was 0.8% for both the 1.5 mg and 2 mg groups and 0.4% for the placebo groups.

A weight loss of 1.2 kg (significant versus baseline) was achieved in the 3 mg group with over 80% of patients losing some weight, versus a 0.4 kg reduction in that trial's placebo group (not significant versus baseline). Weight losses of 2.0 kg and 1.3 kg, respectively, were observed in the 1.5 mg and 2.0 mg dose groups of the first trial (ITT (intent-to-treat) significant versus baseline but not against placebo).

The drug was well tolerated. The drug-related nausea rate across all treatment arms in both trials was 23% versus 10% in the placebo groups; the overall vomiting rate across all treatment arms in both trials was 11% versus 6% in the placebo groups; and the overall diarrhea rate across all treatment arms in both trials was 10% versus 8% in the placebo groups. The incidence of these adverse events diminished over time. As an example, in the highest dose cohort of 3 mg, there was no nausea or vomiting after day 28.

Injection site adverse events were rare and actually occurred less frequently in the treatment groups than the placebo groups.

These data demonstrate that administration of an exendin-4(1-39) Lys⁴⁰ (ε-AEEA-MPA)-NH₂ HSA-conjugate formulation as described in Example 7 and Example 8 results in a robust reduction in HbA1 along with weight loss and excellent GI tolerability. In addition, the liquid formulation and low injection volume (via a very fine gauge needle) caused few injection site reactions. Thus, administration of an exendin-4(1-39) Lys⁴⁰ (ε-AEEA-MPA)-NH₂ HSA-conjugate formulation as described herein presents clear advantages from a patient preference perspective for the treatment of diabetes.

All publications, patents and patent applications cited in this specification are incorporated by reference in their entireties for all purposes, as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications can be made thereto without departing from the spirit or scope of the appended claims.

What is claimed is:

1. A pharmaceutical formulation comprising: a conjugate of albumin and an insulinotropic peptide, said insulinotropic peptide comprising a sequence which has not more than 3 amino acid substitutions, deletions, or insertions relative to the native exendin-4 sequence, said conjugate being at a concentration of about 1 mg/ml to about 100 mg/ml; a buffer; a tonicity modifier, wherein the tonicity modifier is at a concentration of at least 1 mM; a stabilizer; and a surfactant, wherein said formulation has a pH from about 4 to about 8.

2. The pharmaceutical formulation of claim 1 wherein the conjugate comprises albumin cysteine 34 thiol covalently linked to a [2-[2-[2-maleimidopropionamido(ethoxy)ethoxy]acetic acid linker covalently linked to the epsilon amino of a lysine of said peptide.

3. The pharmaceutical formulation of claim 1 wherein the conjugate is according to the following: 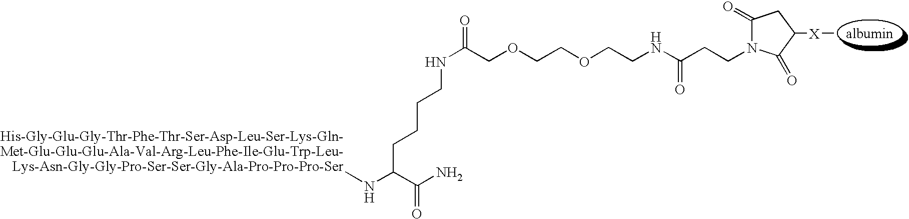
(SEQ ID NO: 33)
wherein X is S, O, or NH of an amino acid of albumin.

4. The pharmaceutical formulation of claim 2 wherein said lysine has been added to the native exendin-4 sequence.

5. The pharmaceutical formulation of claim 2 wherein said lysine has been added to the carboxy terminus of the native exendin-4 sequence.

6. The pharmaceutical formulation of claim 1, wherein the albumin is human serum albumin.

7. The pharmaceutical formulation of claim 1 wherein the albumin is recombinant serum albumin.

8. The pharmaceutical formulation of claim 1 wherein the albumin is recombinant human serum albumin.

9. The pharmaceutical formulation of claim 1 wherein the conjugate comprises recombinant human serum albumin cysteine 34 thiol covalently linked to a [2-[2-[2 maleimidopropionamido(ethoxy)ethoxy]acetic acid linker covalently linked to the epsilon amino of the carboxy terminal lysine of exendin-4(1-39)Lys⁴⁰-NH₂.

10. The pharmaceutical formulation of claim 1, wherein said conjugate is purified.

11. The pharmaceutical formulation of claim 1, wherein said conjugate is at a concentration from about 1 mg/ml to about 50 mg/ml.

12. The pharmaceutical formulation of claim 1, wherein said conjugate is at a concentration from about 1 mg/ml to about 15 mg/ml.

13. The pharmaceutical formulation of claim 1, wherein said conjugate is at a concentration from about 1 mg/ml to about 10 mg/ml.

14. The pharmaceutical formulation of claim 1, wherein said conjugate is at a concentration of about 10 mg/ml.

15. The pharmaceutical formulation of claim 1, wherein said conjugate is at a concentration of about 20 mg/ml.

16. The pharmaceutical formulation of claim 1, wherein the pH is between about 5 and about 7.

17. The pharmaceutical formulation of claim 1, wherein the pH is about 5.0.

18. The pharmaceutical formulation of claim 1, wherein the pH is about 7.0.

19. The pharmaceutical formulation of claim 1, wherein the buffer is an acetate buffer.

20. The pharmaceutical formulation of claim 19, wherein the acetate buffer is a sodium acetate buffer, and wherein the pH is about 4.0 to about 6.0.

21. The pharmaceutical formulation of claim 1, wherein the buffer is a phosphate buffer.

22. The pharmaceutical formulation of claim 21, wherein the phosphate buffer is a sodium phosphate buffer, and wherein the pH is about 6.0 to about 8.0.

23. The pharmaceutical formulation of claim 1, wherein the buffer is at a concentration from 1 mM to about 20 mM.

24. The pharmaceutical formulation of claim 1, wherein the buffer is at a concentration from 5 mM to about 15 mM.

25. The pharmaceutical formulation of claim 1, wherein the buffer is at a concentration at about 10 mM.

26. The pharmaceutical formulation of claim 1, wherein the tonicity modifier is sodium chloride.

27. The pharmaceutical formulation of claim 26, wherein the sodium chloride is at a concentration of about 135 mM to about 155 mM.

28. The pharmaceutical formulation of claim 26, wherein the sodium chloride is at a concentration of about 135 mM.

29. The pharmaceutical formulation of claim 26, wherein the sodium chloride is at a concentration of about 150 mM.

30. The pharmaceutical formulation of claim 1, wherein the tonicity modifier is sorbitol.

31. The pharmaceutical formulation of claim 30, wherein sorbitol is about 5% (w/v).

32. The pharmaceutical formulation of claim 1, wherein the stabilizer is sodium octanoate.

33. The pharmaceutical formulation of claim 32, wherein the sodium octanoate is at a concentration of about 5 mM.

34. The pharmaceutical formulation of claim 1, wherein the surfactant is pluronic F68.

35. The pharmaceutical formulation of claim 34, wherein the pluronic F68 is about 0.1% (w/v).

36. The pharmaceutical formulation of claim 1, wherein the pharmaceutical formulation further comprises a preservative.

37. The pharmaceutical formulation of claim 36, wherein the preservative is selected from the group consisting of methanol, ethanol, iso-propanol, glycerol, resorcinol, 2-methyl-2,4-pentadiol, merthiolate (thimerosal), benzalkonium chloride, and sodium benzoate.

38. The pharmaceutical formulation of claim 1, wherein the pharmaceutical formulation is in a unit dosage form.

39. The pharmaceutical formulation of claim 1, wherein the pharmaceutical formulation is in a multi-use dosage form.

40. The pharmaceutical formulation of claim 1, wherein the pharmaceutical formulation is a liquid dosage form.

41. The pharmaceutical formulation of claim 1, wherein the pharmaceutical formulation is a lyophilized dosage form.

42. The pharmaceutical formulation of claim 1, wherein the pharmaceutical formulation is suitable for parenteral administration.

43. The pharmaceutical formulation of claim 42, wherein the pharmaceutical formulation is suitable for subcutaneous, intravenous, intramuscular, transdermal, intra-arterial, intra-peritoneal, pulmonary or oral administration.

44. The pharmaceutical formulation of claim 42, wherein the pharmaceutical formulation is suitable for subcutaneous administration.

45. The pharmaceutical formulation of claim 1, wherein said conjugate is at a concentration of 10 mg/ml, said buffer is sodium acetate at a concentration of 10 mM, said tonicity modifier is sodium chloride at a concentration of 150 mM, said stabilizer is sodium octanoate at a concentration of 5 mM, said surfactant is pluronic F68 at a concentration of 0.1% (w/v), and wherein said formulation has a pH of about 5.0.

46. The pharmaceutical formulation of claim 1, wherein said conjugate is at a concentration of 10 mg/ml, said buffer is sodium phosphate at a concentration of 10 mM, said tonicity modifier is sodium chloride at a concentration of 135 mM, said stabilizer is sodium octanoate at a concentration of 8 mM, said surfactant is polysorbate 80 at a concentration of 15 mg/L, and wherein said formulation has a pH of about 7.0.

47. A method of treating type II diabetes mellitus in a subject, comprising administering to a subject having type II diabetes mellitus a pharmaceutical formulation comprising: a conjugate of albumin and an insulinotropic peptide, said insulinotropic peptide comprising a sequence which has not more than 3 amino acid substitutions, deletions, or insertions relative to the native exendin-4 sequence, said conjugate being at a concentration of about 1 mg/ml to about 100 mg/ml; a buffer; a tonicity modifier; a stabilizer; and a surfactant, wherein said formulation has a pH from about 4 to about 8.

48. A method of treating type II diabetes mellitus in a subject, comprising administering to a subject having type II diabetes mellitus the pharmaceutical formulation of claim 45.

49. A method of treating type II diabetes mellitus in a subject, comprising administering to a subject having type II diabetes mellitus the pharmaceutical formulation of claim 46.

50. The method of claim 48, which comprises administering about 1.0 to 4.0 mg of the conjugate to the subject per week.

51. The method of claim 48, which comprises administering about 1.5 to 2.0 mg of the conjugate to the subject per week.

52. The method of claim 48, which comprises administering about 3.0 to 4.0 mg of the conjugate to the subject per week.

53. The method of claim 48, which comprises administering 1.5 mg of the conjugate to the subject once a week.

54. The method of claim 48, which comprises administering 2.0 mg of the conjugate to the subject once a week.

55. The method of claim 48, which comprises administering 3.0 mg of the conjugate to the subject once a week.

56. The method of claim 48, which comprises administering 1.5 mg of the conjugate to the subject twice a week.

57. The method of claim 48, comprising the following steps in the order stated:
(a) administering 1.5 mg of the conjugate to the subject once a week for a first duration of time; and
(b) administering 2.0 mg of the conjugate to the subject once a week for a second duration of time.

58. The method of claim 57, wherein the first duration of time is 4 weeks, and wherein the second duration of time is 8 weeks.

59. The method of claim 48, comprising the following steps in the order stated:
(a) administering 1.5 mg of the conjugate to the subject twice a week for a first duration of time; and
(b) administering 2.0 mg of the conjugate to the subject twice a week for a second duration of time.

60. The method of claim 59, wherein the first duration of time is 4 weeks.

61. The method of claim 48, comprising the following steps in the order stated:
(a) administering 1.5 mg of the conjugate to the subject once a week for a first duration of time;
(b) administering 2.0 mg of the conjugate to the subject once a week for a second duration of time; and
(c) administering 3.0 mg of the conjugate to the subject once a week for a third duration of time.

62. The method of claim 61, wherein the first duration of time is 4 weeks, and wherein the second duration of time is 4 weeks.

63. The method of claim 61, wherein the first duration of time is 2 weeks, and wherein the second duration of time is 2 weeks.

64. A method of treating type II diabetes mellitus in a subject, comprising administering to a subject having type II diabetes mellitus a pharmaceutical formulation comprising an insulinotropic conjugated exendin-4 derivative, the derivative comprising recombinant human serum albumin cysteine 34 thiol covalently linked to a [2-[2-[2 maleimidopropionamido(ethoxy)ethoxy]acetic acid linker covalently linked to the epsilon amino of the carboxy terminal lysine of exendin-4(1-39)Lys⁴⁰-NH₂, wherein the subject is administered 1.5 mg of the conjugated exendin-4 derivative once a week.

65. A method of treating type II diabetes mellitus in a subject, comprising administering to a subject having type II diabetes mellitus a pharmaceutical formulation comprising an insulinotropic conjugated exendin-4 derivative, the derivative comprising recombinant human serum albumin cysteine 34 thiol covalently linked to a [2-[2-[2 maleimidopropionamido(ethoxy)ethoxy]acetic acid linker covalently linked to the epsilon amino of the carboxy terminal lysine of exendin-4(1-39)Lys⁴⁰-NH₂, wherein the subject is administered 1.5 mg of the conjugated exendin-4 derivative twice a week.

66. A method of treating type II diabetes mellitus in a subject, comprising administering to a subject having type II diabetes mellitus a pharmaceutical formulation comprising an insulinotropic conjugated exendin-4 derivative, the derivative comprising recombinant human serum albumin cysteine 34 thiol covalently linked to a [2-[2-[2 maleimidopropionamido(ethoxy)ethoxy]acetic acid linker covalently linked to the epsilon amino of the carboxy terminal lysine of exendin-4(1-39)Lys⁴⁰-NH₂, wherein the subject is administered 2.0 mg of the conjugated exendin-4 derivative once a week.

67. A method of treating type II diabetes mellitus in a subject, comprising administering to a subject having type II diabetes mellitus a pharmaceutical formulation comprising an insulinotropic conjugated exendin-4 derivative, the derivative comprising recombinant human serum albumin cysteine 34 thiol covalently linked to a [2-[2-[2 maleimidopropionamido(ethoxy)ethoxy]acetic acid linker covalently linked to the epsilon amino of the carboxy terminal lysine of exendin-4(1-39)Lys⁴⁰-NH₂, wherein the subject is administered 2.0 mg of the conjugated exendin-4 derivative twice a week.

68. A method of treating type II diabetes mellitus in a subject, comprising administering to a subject having type II diabetes mellitus a pharmaceutical formulation comprising an insulinotropic conjugated exendin-4 derivative, the derivative comprising recombinant human serum albumin cysteine 34 thiol covalently linked to a [2-[2-[2 maleimidopropionamido(ethoxy)ethoxy]acetic acid linker covalently linked to the epsilon amino of the carboxy terminal lysine of exendin-4(1-39)Lys⁴⁰-NH₂, wherein the subject is administered 3.0 mg of the conjugated exendin-4 derivative once a week.

69. A method of treating type II diabetes mellitus in a subject, comprising administering to a subject having type II diabetes mellitus a pharmaceutical formulation comprising an insulinotropic conjugated exendin-4 derivative, the derivative comprising recombinant human serum albumin cysteine 34 thiol covalently linked to a [2-[2-[2 maleimidopropionamido(ethoxy)ethoxy]acetic acid linker covalently linked to the epsilon amino of the carboxy terminal lysine of exendin-4(1-39)Lys⁴⁰-NH₂, wherein the subject is administered 1.5 mg of the conjugated exendin-4 derivative once a week for 4 weeks followed by 2.0 mg of the conjugated exendin-4 derivative once a week.

70. A method of treating type II diabetes mellitus in a subject, comprising administering to a subject having type II diabetes mellitus a pharmaceutical formulation comprising an insulinotropic conjugated exendin-4 derivative, the derivative comprising recombinant human serum albumin cysteine 34 thiol covalently linked to a [2-[2-[2 maleimidopropionamido(ethoxy)ethoxy]acetic acid linker covalently linked to the epsilon amino of the carboxy terminal lysine of exendin-4(1-39)Lys⁴⁰-NH₂, wherein the subject is administered 1.5 mg of the conjugated exendin-4 derivative twice a week for 4 weeks followed by 2.0 mg of the conjugated exendin-4 derivative once a week.

71. A method of treating type II diabetes mellitus in a subject, comprising administering to a subject having type II diabetes mellitus a pharmaceutical formulation comprising an insulinotropic conjugated exendin-4 derivative, the derivative comprising recombinant human serum albumin cysteine 34 thiol covalently linked to a [2-[2-[2 maleimidopropionamido(ethoxy)ethoxy]acetic acid linker covalently linked to the epsilon amino of the carboxy terminal lysine of exendin-4(1-39)Lys⁴⁰-NH₂, wherein the subject is administered 1.5 mg of the conjugated exendin-4 derivative twice a week for 4 weeks followed by 2.0 mg of the conjugated exendin-4 derivative twice a week.

72. A method of treating type II diabetes mellitus in a subject, comprising administering to a subject having type II diabetes mellitus a pharmaceutical formulation comprising an insulinotropic conjugated exendin-4 derivative, the derivative comprising recombinant human serum albumin cysteine 34 thiol covalently linked to a [2-[2-[2 maleimidopropionamido(ethoxy)ethoxy]acetic acid linker covalently linked to the epsilon amino of the carboxy terminal lysine of exendin-4(1-39)Lys⁴⁰-NH₂, wherein the subject is administered 1.5 mg of the conjugated exendin-4 derivative once a week for 4 weeks, followed by 2.0 mg of the conjugated exendin-4 derivative once a week for 4 weeks, followed by 3.0 mg of the conjugated exendin-4 derivative once a week.

73. A method of treating type II diabetes mellitus in a subject, comprising administering to a subject having type II diabetes mellitus a pharmaceutical formulation comprising an insulinotropic conjugated exendin-4 derivative, the derivative comprising recombinant human serum albumin cysteine 34 thiol covalently linked to a [2-[2-[2 maleimidopropionamido(ethoxy)ethoxy]acetic acid linker covalently linked to the epsilon amino of the carboxy terminal lysine of exendin-4(1-39)Lys⁴⁰-NH₂, wherein the subject is administered 1.5 mg of the conjugated exendin-4 derivative once a week for 2 weeks, followed by 2.0 mg of the conjugated exendin-4 derivative once a week for 2 weeks, followed by 3.0 mg of the conjugated exendin-4 derivative once a week.

74. A kit for the treatment of type II diabetes mellitus in a subject, comprising one or more containers comprising the pharmaceutical formulation of claim 1.

75. The kit of claim 74, wherein said one or more containers each comprise a unit dosage form of the pharmaceutical formulation.

76. The kit of claim 74, wherein the pharmaceutical formulation is lyophilized.

77. The kit of claim 74, wherein the lyophilized pharmaceutical formulation is produced by lyophilizing in the presence of a non-reducing sugar.

78. The kit of claim 74, wherein the non-reducing sugar is sucrose or trehalose.

79. The kit of claim 76, further comprising one or more containers comprising a sterile diluent for reconstituting the lyophilized pharmaceutical formulation.

80. The method of claim 47, wherein the subject is on a stable dose of ≧1000 mg metformin daily for at least 3 months.

81. A pharmaceutical formulation consisting of a conjugate of albumin and an insulinotropic peptide, said insulinotropic peptide comprising a sequence which has not more than 3 amino acid substitutions, deletions, or insertions relative to the native exendin-4 sequence, said conjugate being at a concentration of about 1 mg/ml to about 100 mg/ml; a buffer; a tonicity modifier; a stabilizer; and a surfactant, wherein said formulation has a pH has a pH from about 4.0 to about 8.0.

82. A pharmaceutical formulation consisting of
(a) conjugate according to the following: 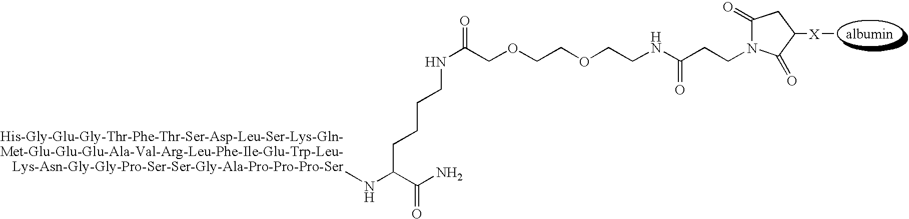
(SEQ ID NO: 33) wherein X is S of cysteine 34 of albumin, said conjugate being at a concentration of 10 mg/ml;
(b) a buffer, wherein said buffer is sodium acetate at a concentration of 10 mM;
(c) a tonicity modifier, wherein said tonicity modifier is sodium chloride at a concentration of 150 mM;
(d) a stabilizer, wherein said stabilizer is sodium octanoate at a concentration of 5 mM; and
(e) a surfactant, wherein said surfactant is pluronic F68 at a concentration of 0.1% (w/v),
wherein said formulation has a pH has a pH of about 5.0.

83. A pharmaceutical formulation consisting of:
(a) conjugate according to the following: 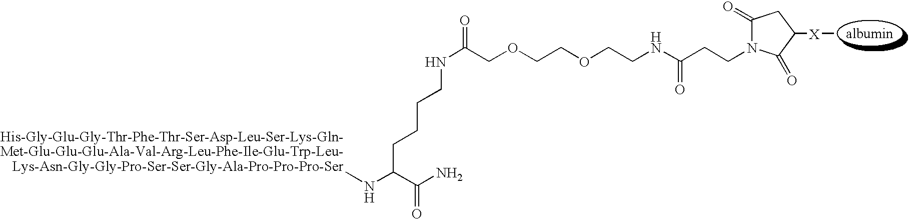
(SEQ ID NO: 33) wherein X is S of cysteine 34 of albumin, said conjugate being at a concentration of 10 mg/ml;
(b) a buffer, wherein said buffer is sodium phosphate at a concentration of 10 mM;
(c) a tonicity modifier, wherein said tonicity modifier is sodium chloride at a concentration of 135 mM;
(d) a stabilizer, wherein said stabilizer is sodium octanoate at a concentration of 8 mM; and
(e) a surfactant, wherein said surfactant is polysorbate 80 at a concentration of 15 mg/L,
wherein said formulation has a pH of about 7.0.

84. The method of any one of claims 47, wherein the albumin is human serum albumin.

85. The method of any one of claims 47, wherein the subject is a human.
